(19) United States
(12) Patent Application Publication
(10) Pub. No.: US 2012/0277766 A1
(43) Pub. Date: Nov. 1, 2012

(54) APPARATUS AND METHODS FOR CLOSURE OF FISSURES IN THE ANULUS FIBROSIS

(75) Inventor: Bret A. Ferree, Cincinnati, OH (US)

(73) Assignee: Anova Corporation

(21) Appl. No.: 13/297,789

(22) Filed: Nov. 16, 2011

Related U.S. Application Data
(60) Provisional application No. 61/414,186, filed on Nov. 16, 2010.

(51) Int. Cl. A61B 17/04

(52) U.S. Cl. 606/144

(57) ABSTRACT

Apparatus for repairing a defect in an anulus fibrosis surrounding an intradiscal space, the apparatus comprising a first surgical instrument having a distal end adapted for penetration through an annulus fibrosis on one side of a defect in the annulus fibrosis such that the distal end of the first surgical instrument enters into an intradiscal space; a flexible longitudinal fixation component temporarily coupled to the distal end of the first surgical instrument; and a second surgical instrument having a distal end adapted for penetration through an annulus fibrosis on the other side of a defect in the annulus fibrosis or through a defect in the annulus fibrosis, the distal end of the second surgical instrument including a device for capturing the flexible longitudinal fixation component within an intradiscal space and pulling the flexible longitudinal fixation component so as to repair the defect.

### REFERENCE TO RELATED APPLICATION

This application claims priority to U.S. Provisional Patent Application Ser. No. 61/414,186, filed Nov. 16, 2010, the entire content of which is incorporated herein by reference.

### FIELD OF THE INVENTION

This invention relates generally to the treatment of intervertebral disc herniation and degenerative disc disease and, in particular, to apparatus and methods for closure of fissures in the anulus fibrosis.

### BACKGROUND OF THE INVENTION

The human intervertebral disc is an oval to kidney bean-shaped structure of variable size depending on the location in the spine. The outer portion of the disc is known as the anulus fibrosus (AF, also known as the “anulus fibrosis”). The AF is made of ten to twenty collagen fiber lamellae. The collagen fibers within a lamella are parallel. Successive lamellae are oriented in alternating directions. About 48 percent of the lamellae are incomplete, but this value varies based upon location and increases with age. On average, the lamellae lie at an angle of sixty degrees with respect to the vertebral axis line, but this too varies depending upon location. The orientation serves to control vertebral motion (one half of the bands tighten to check motion when the vertebra above or below the disc are turned in either direction).

The anulus fibrosus contains the nucleus pulposus (NP). The nucleus pulposus serves to transmit and dampen axial loads. A high water content (approximately 70-80 percent) assists the nucleus in this function. The water content has a diurnal variation. The nucleus imbibes water while a person lies recumbent. Nuclear material removed from the body and placed into water will imbibe water swelling to several times its normal size. Activity squeezes fluid from the disc. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per microliter.

The intervertebral disc changes or “degenerates” with age. As a person ages, the water content of the disc falls from approximately 85 percent at birth to approximately 70 percent in the elderly. The ratio of chondroitin sulfate to keratin sulfate decreases with age, while the ratio of chondroitin 6 sulfate to chondroitin 4 sulfate increases with age. The distinction between the anulus and the nucleus decreases with age. Generally disc degeneration is painless.

Premature or accelerated disc degeneration is known as degenerative disc disease. A large portion of patients suffering from chronic low back pain are thought to have this condition. As the disc degenerates, the nucleus and anulus functions are compromised. The nucleus becomes thinner and less able to handle compression loads. The anulus fibers become redundant as the nucleus shrinks. The redundant anular fibers are less effective in controlling vertebral motion. This disc pathology can result in: 1) bulging of the anulus into the spinal cord or nerves; 2) narrowing of the space between the vertebra where the nerves exit; 3) tears of the anulus as abnormal loads are transmitted to the anulus and the anulus is subjected to excessive motion between vertebra; and 4) disc herniation or extrusion of the nucleus through complete anular tears.

Current surgical treatments for disc degeneration are destructive. One group of procedures, which includes lumbar discectomy, removes the nucleus or a portion of the nucleus. A second group of procedures destroy nuclear material. This group includes Chymopapin (an enzyme) injection, laser discectomy, and thermal therapy (heat treatment to denature proteins). The first two groups of procedures compromise the treated disc. A third group, which includes spinal fusion procedures, either removes the disc or the disc's function by connecting two or more vertebra together with bone. Fusion procedures transmit additional stress to the adjacent discs, which results in premature disc degeneration of the adjacent discs. These destructive procedures lead to acceleration of disc degeneration.

Prosthetic disc replacement offers many advantages. The prosthetic disc attempts to eliminate a patient's pain while preserving the disc's function. Current prosthetic disc implants either replace the nucleus or replace both the nucleus and the anulus. Both types of current procedures remove the degenerated disc component to allow room for the prosthetic component. Although the use of resilient materials has been proposed, the need remains for further improvements in the way in which prosthetic components are incorporated into the disc space to ensure strength and longevity. Such improvements are necessary, since the prosthesis may be subjected to 100,000,000 compression cycles over the life of the implant.

Current nucleus replacements (NRs) may cause lower back pain if too much pressure is applied to the anulus fibrosus. As discussed in co-pending U.S. Pat. Nos. 6,878,167 and 7,201,774, the content of each being expressly incorporated herein by reference in their entirety, the posterior portion of the anulus fibrosus has abundant pain fibers.

Herniated nucleus pulposus (HNP) occurs from tears in the anulus fibrosus. The herniated nucleus pulposus often allies pressure on the nerves or spinal cord. Compressed nerves cause back and leg or arm pain. Although a patient's symptoms result primarily from pressure by the nucleus pulposus, the primary pathology lies in the anulus fibrosus.

Surgery for herniated nucleus pulposus, known as microlumbar discectomy (MLD), only addresses the nucleus pulposus. The opening in the anulus fibrosus is enlarged during surgery, further weakening the anulus fibrosus. Surgeons also remove generous amounts of the nucleus pulposus to reduce the risk of extruding additional pieces of nucleus pulposus through the defect in the anulus fibrosus. Although microlumbar discectomy decreases or eliminates a patient's leg or arm pain, the procedure damages weakened discs.

### SUMMARY OF THE INVENTION

This invention broadly facilitates reconstruction of the anulus fibrosus (AF) and the nucleus pulposus (NP). Such reconstruction may prevent recurrent herniation following microlumbar discectomy. The invention may also be used in the treatment of herniated discs, anular tears of the disc, or disc degeneration, while enabling surgeons to preserve the contained nucleus pulposus. The methods and apparatus may be used to treat discs throughout the spine including the cervical, thoracic, and lumbar spines of humans and animals.

The invention also enables surgeons to reconstruct the anulus fibrosus and replace or augment the nucleus pulposus. Novel nucleus replacements (NR) may be added to the disc. Anulus reconstruction prevents extrusion of the nucleus replacements through holes in the anulus fibrosus. The nucleus replacements and the anulus fibrosus reconstruction prevent excessive pressure on the anulus fibrosus that may cause back or leg pain. The nucleus replacements may be made of natural or synthetic materials. Synthetic nucleus replacements may be made of, but are not limited to, polymers including polyurethane, silicon, hydrogel, or other elastomers.

The invention is related to FIGS. 4A-4O of my co-pending U.S. patent application Ser. No. 61/305,683 entitled “Intervertebral Disc Treatment Methods and Apparatus,” the entire content of which is incorporated herein by reference. Preferred embodiments of the invention include one or more magnetic components that are used to pass one or more flexible longitudinal fixation components across a soft tissue defect, such as a fissure in the AF. The flexible longitudinal fixation components preferably pass over the inlet of the fissure, which is adjacent to the NP and the outlet of the fissure, which is the exterior of the IVD and is adjacent to the nerves in the spinal canal. The flexible longitudinal fixation component may be anchored to one of the upper and lower vertebral bodies.

Portions of the flexible longitudinal fixation components, such as the ends, are passed through the AF with novel instruments that generally have magnet components. Generally the distal end of the flexible longitudinal fixation component is passed through a cable loop, which includes a generally cylinder magnet or a generally cylindrical ferromagnetic component, then one end of the cable loop with the magnetic component is passed through a surgically created aperture in the AF adjacent to a fissure. A tool with a generally cylindrical magnet is then inserted through the fissure to attract the magnetic component in the cable loop then end of the cable loop with the magnetic component is pulled through the fissure. The cable loop is then pulled from the IVD, which pulls a portion of the flexible longitudinal fixation component in an inside to outside direction or an outside to inside direction, through one or more holes in the AF tissue adjacent to the fissure. A portion of the flexible longitudinal fixation component generally courses parallel to the inner layer of the AF. The polarity of the magnets is preferably axial, which generally causes end to end attachment of the cylindrical magnets.

We discovered that nucleus tissue trapped in fissures of the disc prevent healing. Prior to our discovery, it was believed that poor blood supply to the disc prevented healing of the anulus. We also discovered that the blood supply to the AF increases with AF injury and new blood vessels grow from the exterior of the AF to nearly the nucleus pulposus. Our discoveries lead us to invent devices and techniques to appose the sides of fissures or other anulus defects from the inner layers to the outer layers of the AF. Surgeons rarely attempt closure of the anular defects and when closure was attempted, it was limited to closure of only the outer layers of the anulus.

In particular, we were surprised to discover that closure of only the outer portion of fissures or defects in the AF limits healing to only the outer portion of the AF, at most. In fact, we and others have observed that healing is frequently limited to connective tissue over the AF rather than healing of the anulus. We were also surprised to discover that closure of only the outer layers of layers of the AF may increase accumulation of nucleus tissue trapped in the aperture of the anulus inside the closed outer portion of the anulus, which causes larger, permanent cyst-like defects in the anulus than if the aperture was not closed.

The inventions disclosed in this application and many of my other applications permit full thickness closure of anular defects or fissures in the very limited space of the spinal canal. Generally, such inventions include one or more flexible longitudinal fixation components that pass through all layers of the anulus on two sides of fissures in the anulus. Such flexible longitudinal fixation components generally have two vertical and two horizontal segments. A first horizontal segment lies inside the inner most layer of the AF and it is generally parallel to the inner surface of the AF. The second horizontal segment lies outside the outer layer of the AF and it is generally parallel to the outer surface of the AF. The vertical portions of the flexible longitudinal fixation component are generally perpendicular to the horizontal portions of the flexible fixation component and the vertical portions of the flexible fixation component pass through all 15 to 25 layers or lamellae of the AF. The distance between the vertical portions of the flexible fixation component at the exterior of the disc is preferably less than or equal to the distance between the vertical portions of the flexible fixation component at the inner surface of the AF.

The invention generally passes a tool and one or more ends of flexible components temporarily through the fissure or defect in the anulus, often more than once. Our discovery also led us to weld flexible longitudinal fixation components in our preferred embodiments of the invention. Knots inevitably slip, which loosens the tension on the closed tissues. Decreased tension across fissures in the AF allows NP tissue to migrate into such fissures. Welded flexible longitudinal fixation components are not subject to creep or slippage and therefore maintain tension on the closure, which prevents migration of even small amounts of NP tissue into fissures in the AF. The invention could be used to close other soft tissue defects in the bodies of humans or animals.

### BRIEF DESCRIPTION OF THE DRAWINGS

FIG. 1A is a lateral view of the preferred embodiment of the invention;

FIG. 1B is a lateral view of a longitudinal cross section of the embodiment of the invention drawn in FIG. 1A;

FIG. 1C is an anterior view of the embodiment of the invention drawn in FIG. 1B;

FIG. 2 is an oblique lateral view of an alternative embodiment of the invention drawn in FIG. 1A;

FIG. 3 is a lateral view of the embodiment of the invention drawn in FIG. 1A;

FIG. 4 is an oblique lateral view of an alternative embodiment of the invention drawn in FIG. 3;

FIG. 5A is an anterior view of an alternative embodiment of the invention drawn in FIG. 3;

FIG. 5B is an anterior view of a longitudinal cross section of the embodiments of the invention drawn in FIGS. 2 and 5A;

FIG. 6A is a lateral view of the distal end of the embodiments of the invention drawn in FIGS. 1A and 5A and a superior view of an axial cross section of a portion of the AF;

FIG. 6B is a lateral view of the embodiment of the invention drawn in FIG. 6A and a superior view of an axial cross section of a portion of the AF;

FIG. 6C is a lateral view of the embodiments of the invention drawn in FIGS. 3, 4, and 6B and a superior view of an axial cross section of a portion of the AF;

FIG. 6D is a lateral view of the embodiment of the invention drawn in FIG. 6C and a superior view of an axial cross section of a portion of the AF;

FIG. 6E is a lateral view of the embodiment of the invention drawn in FIG. 6D and a superior view of an axial cross section of a portion of the AF;

FIG. 6F is a lateral view of the embodiment of the invention drawn in FIG. 6E and a superior view of an axial cross section of a portion of the AF;

FIG. 6G is a lateral view of the embodiment of the invention drawn in FIG. 6F and a superior view of an axial cross section of a portion of the AF;

FIG. 6H is a lateral view of the embodiment of the invention drawn in FIG. 6G and a superior view of an axial cross section of a portion of the AF;

FIG. 6I is a posterior view of the embodiment of the invention drawn in FIG. 6H and an IVD;

FIG. 6J is a posterior view of the embodiment of the invention drawn in FIG. 6I and an IVD;

FIG. 6K is a lateral view of embodiment of the invention drawn in FIG. 6J and a superior view of an axial cross section of a portion of the AF;

FIG. 6L is a lateral view of an alternative embodiment of the invention drawn in FIG. 6K and a superior view of a portion of the AF;

FIG. 7 is an oblique lateral view of an alternative embodiment of the invention drawn in 2;

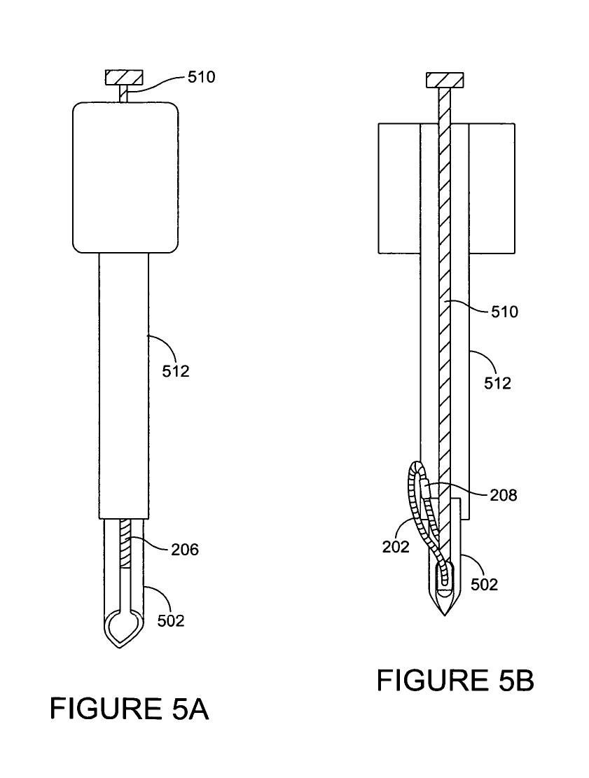

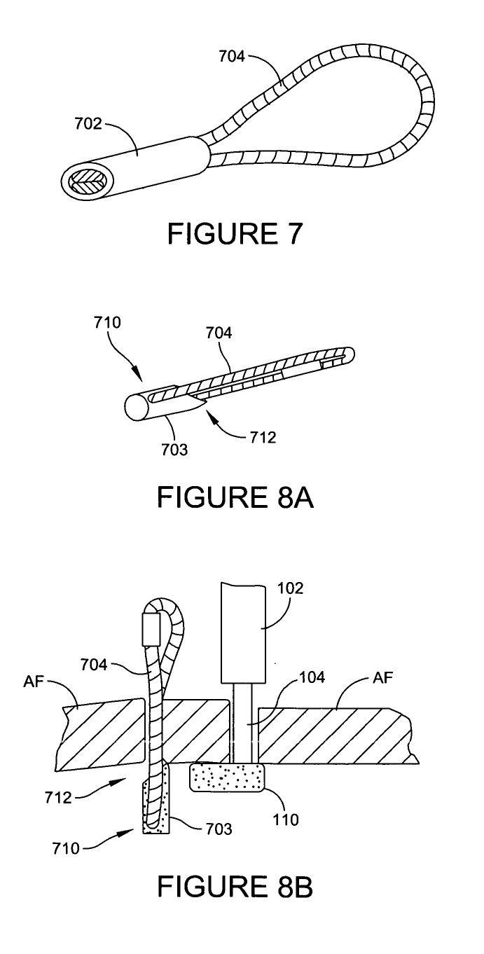

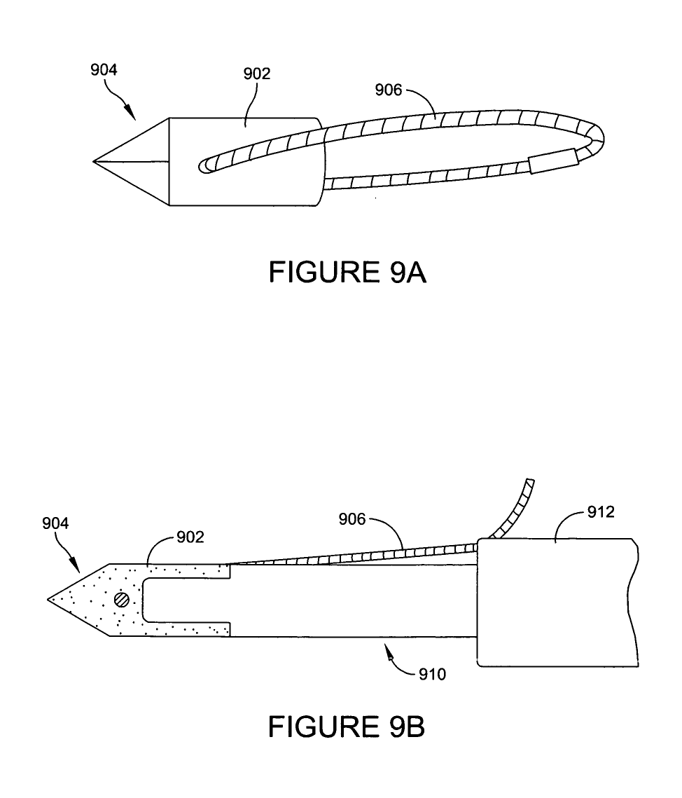

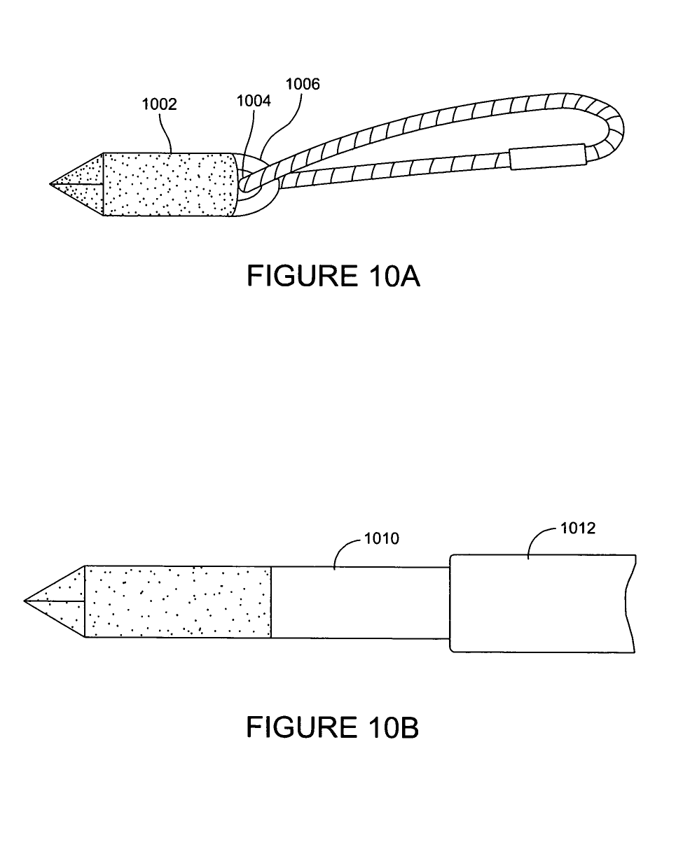

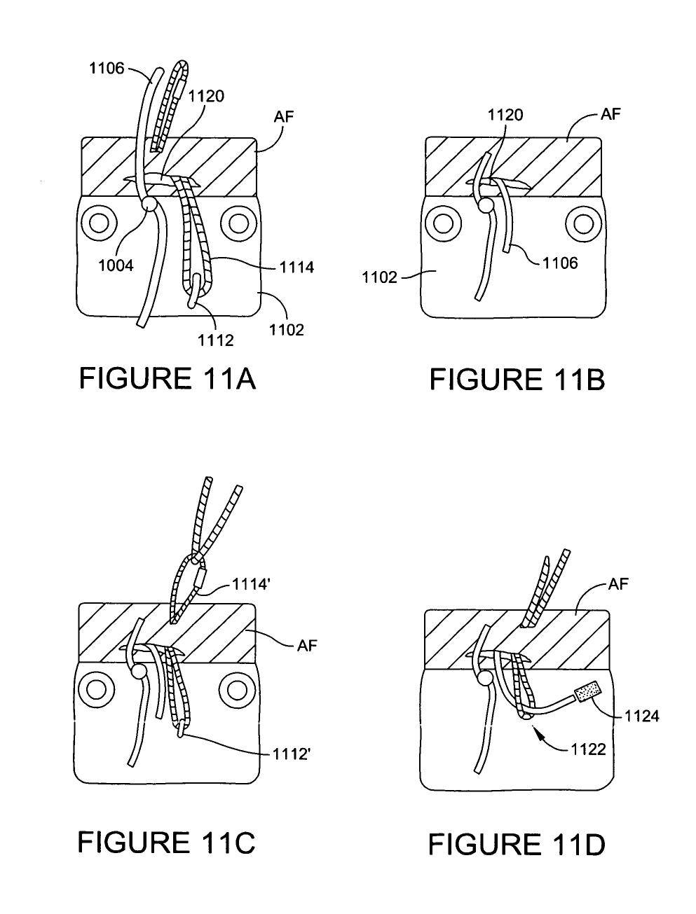

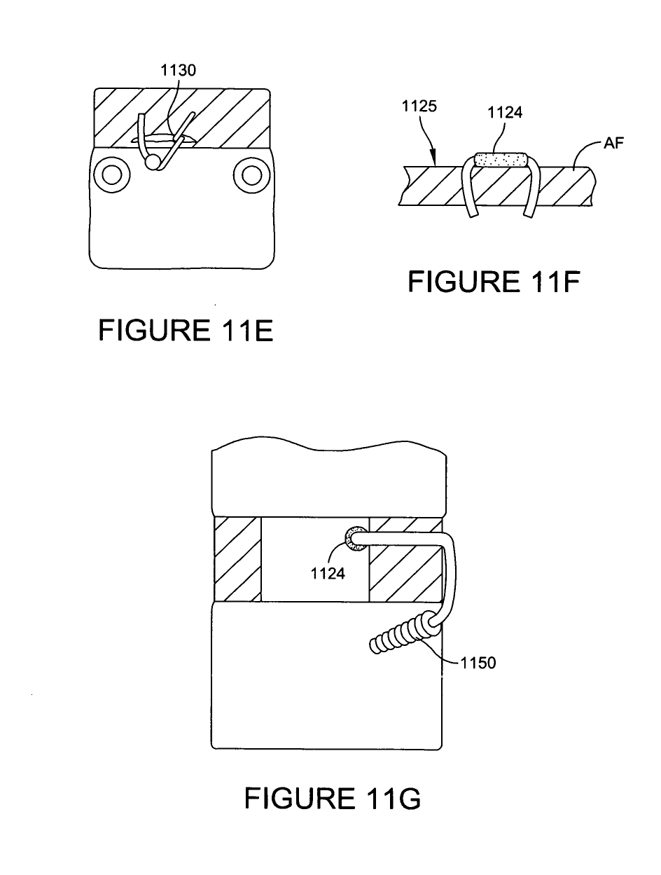

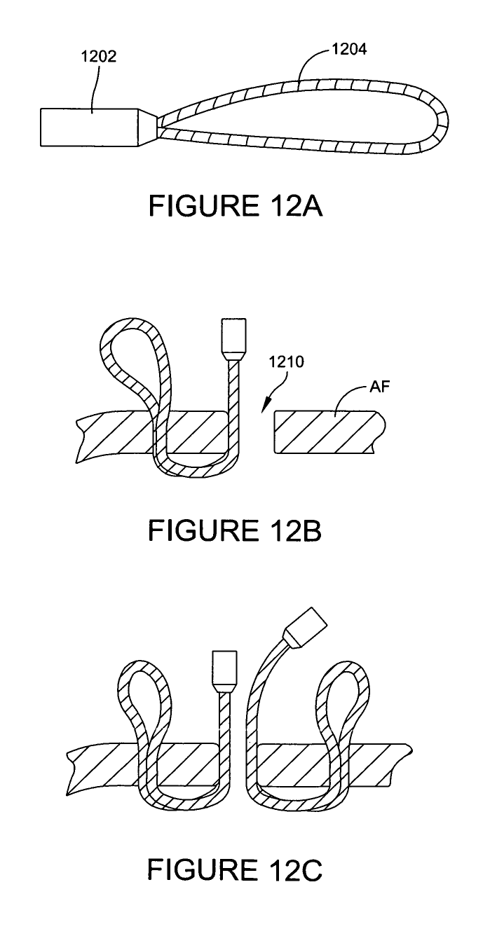

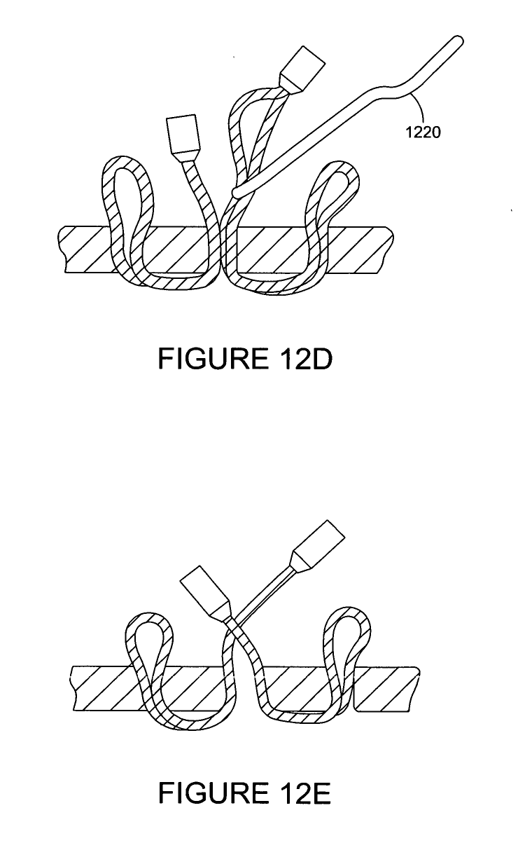

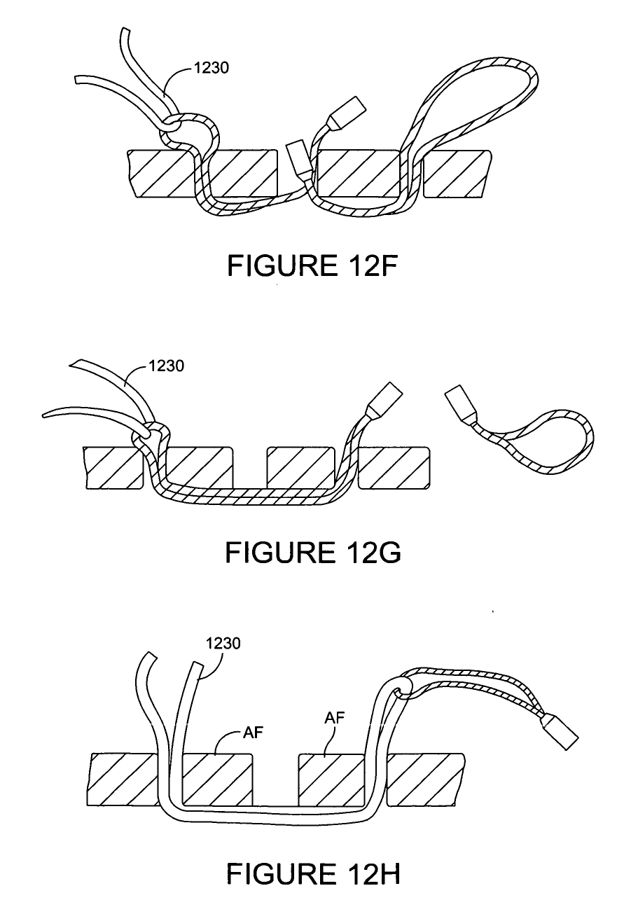

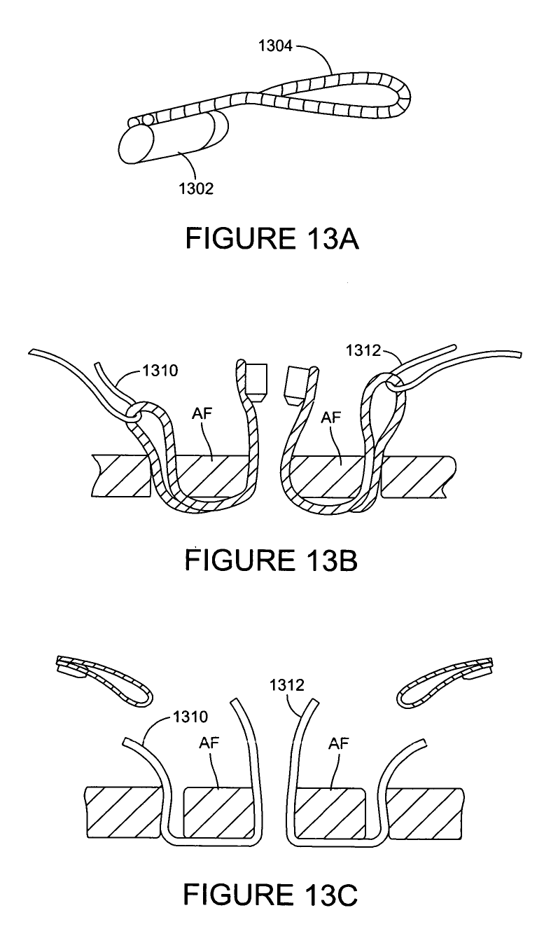

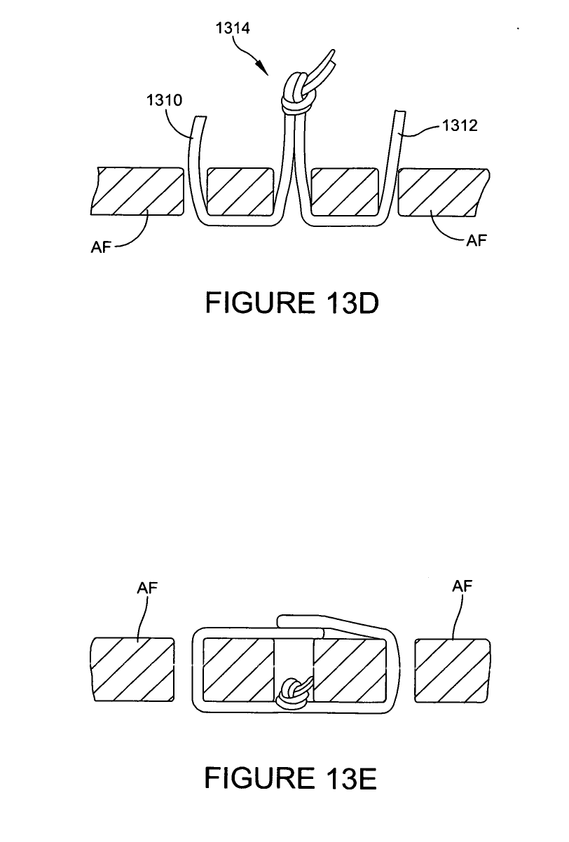

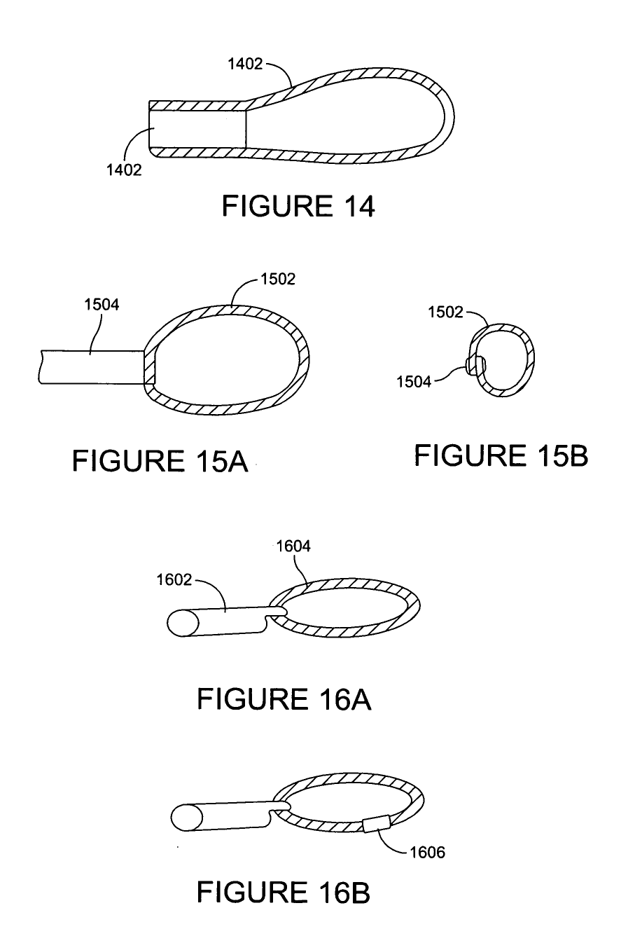

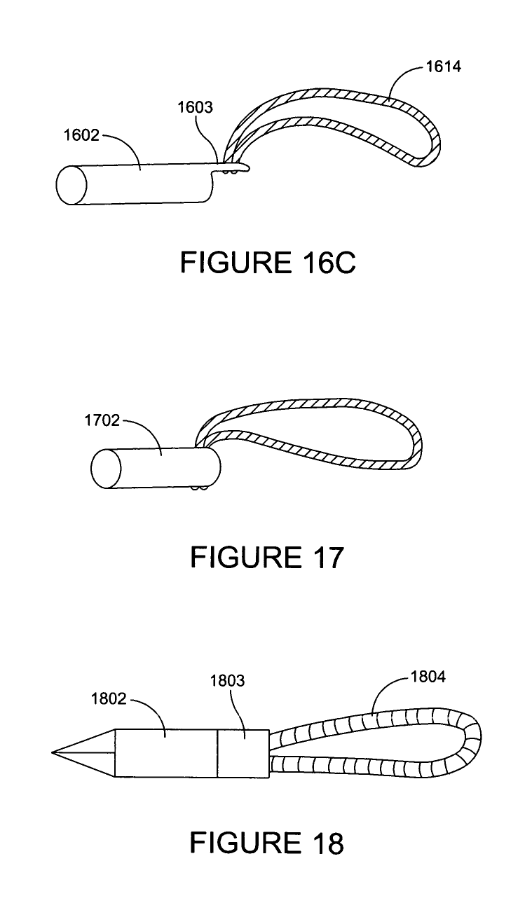

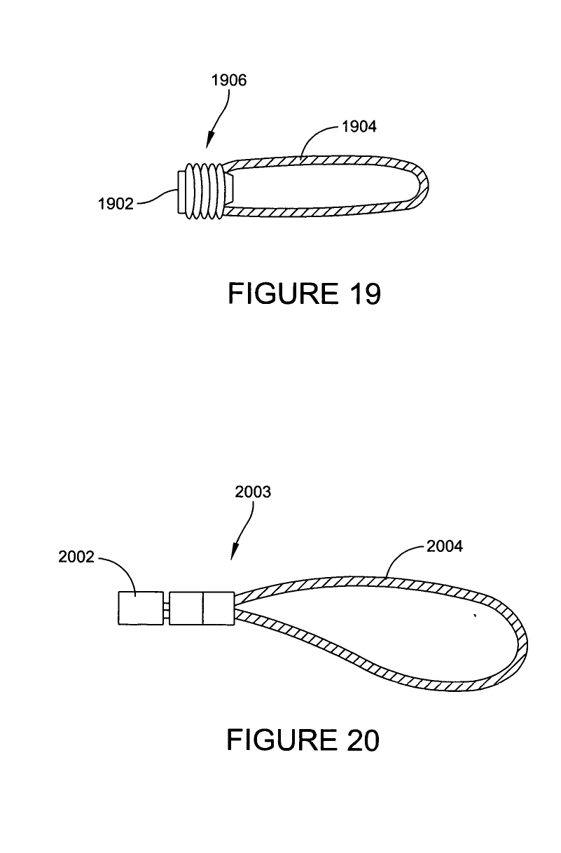

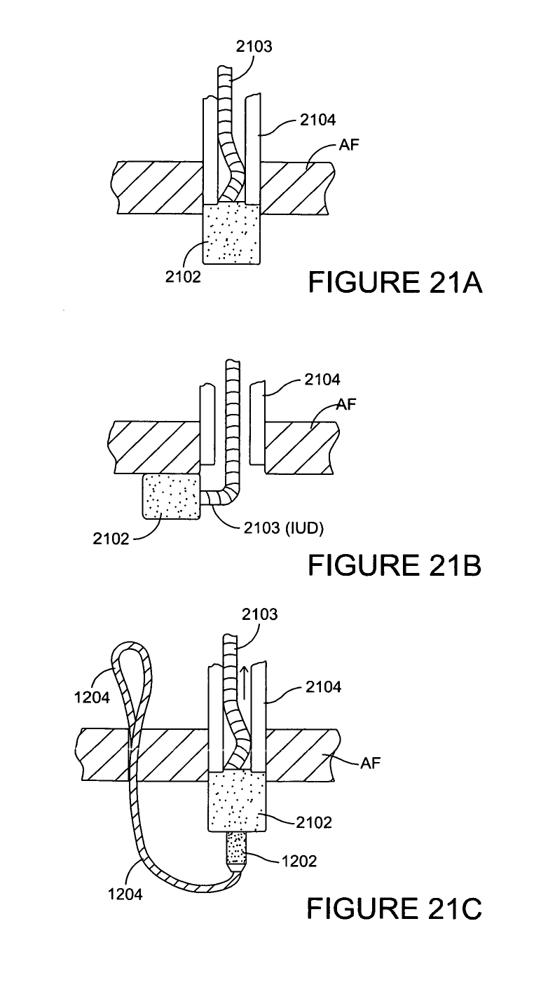

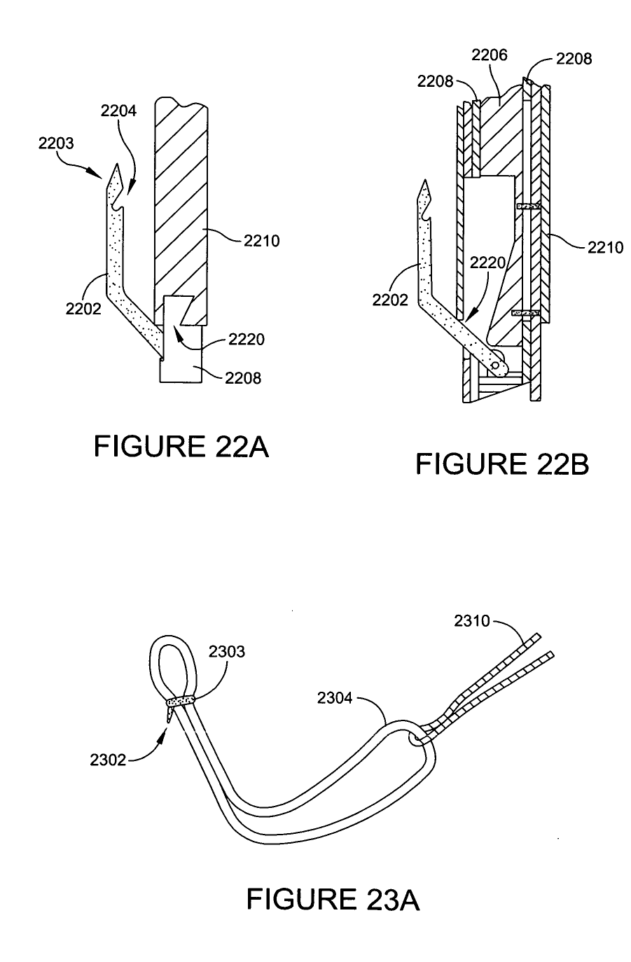

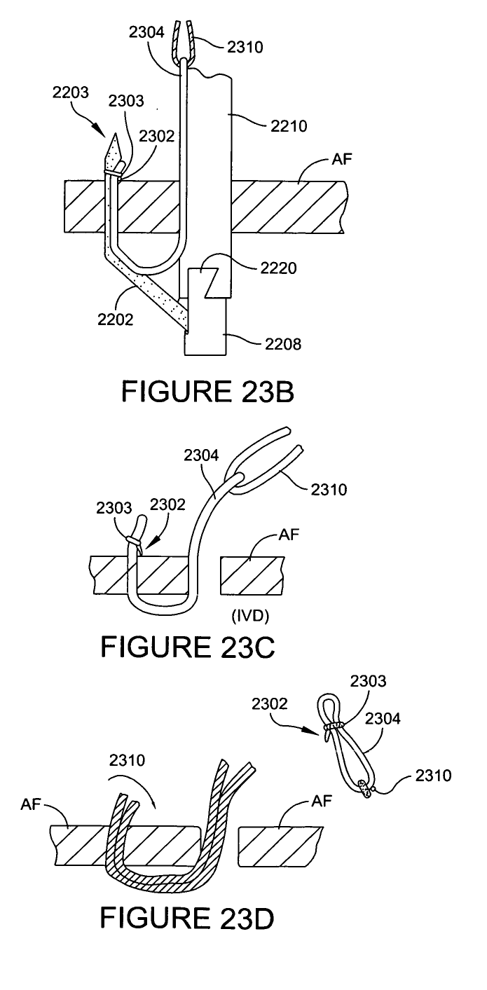

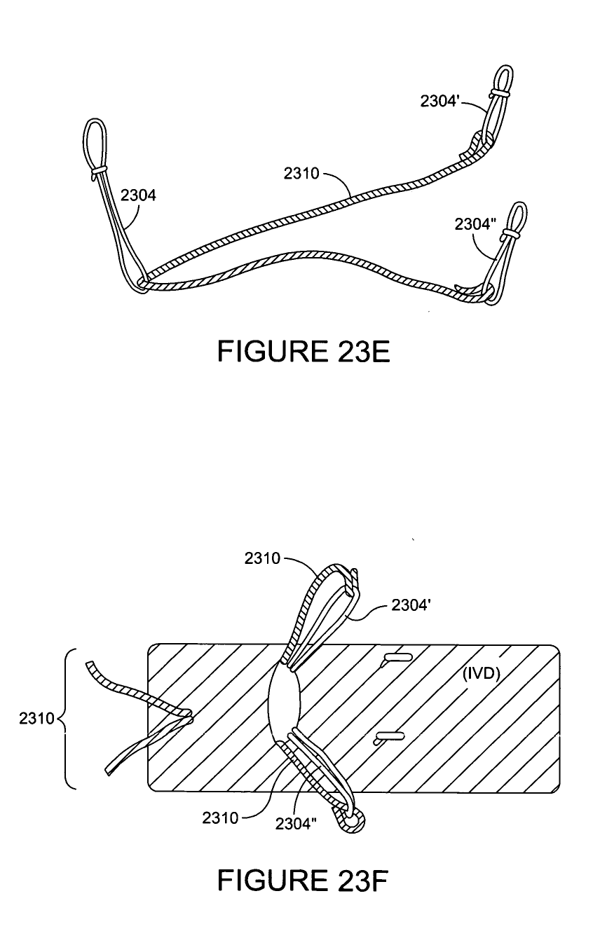

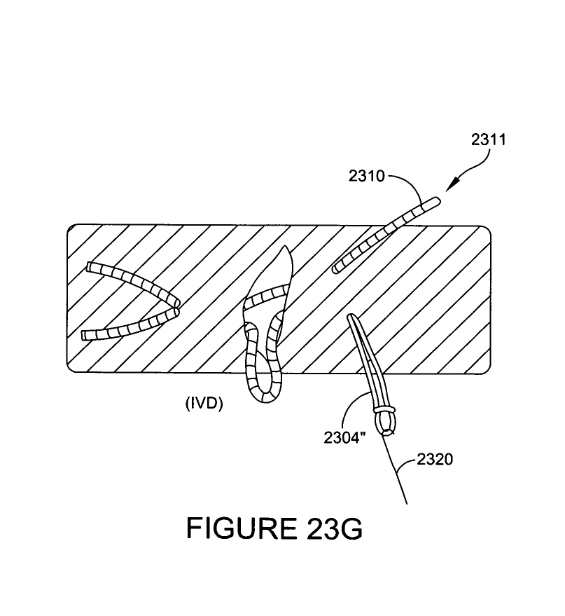

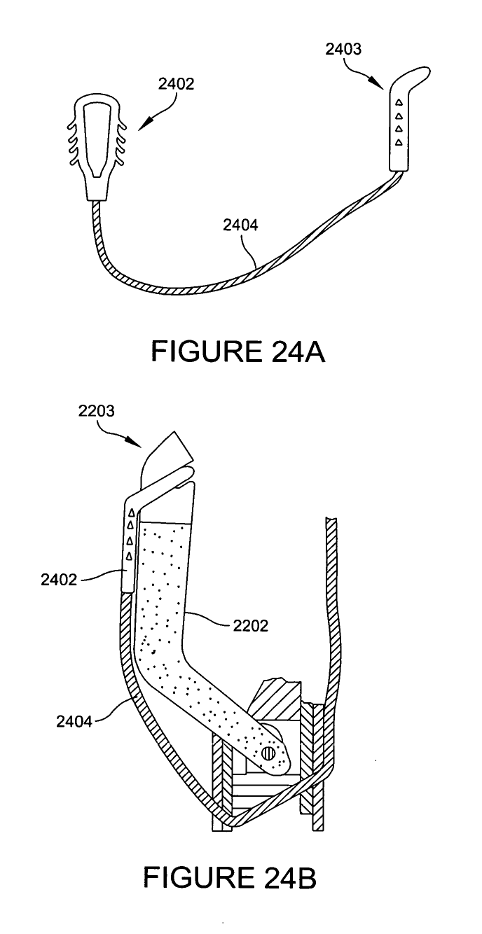

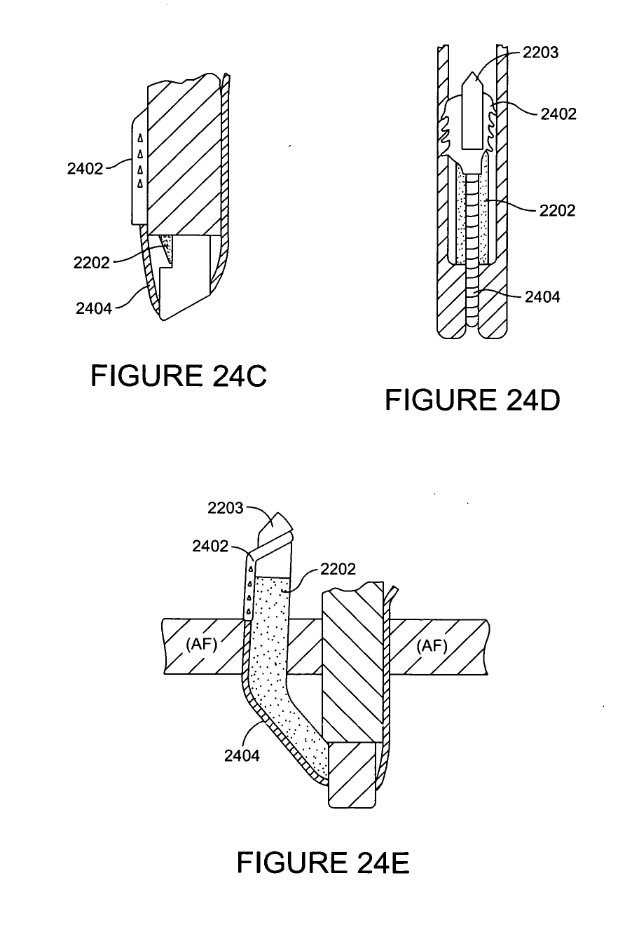

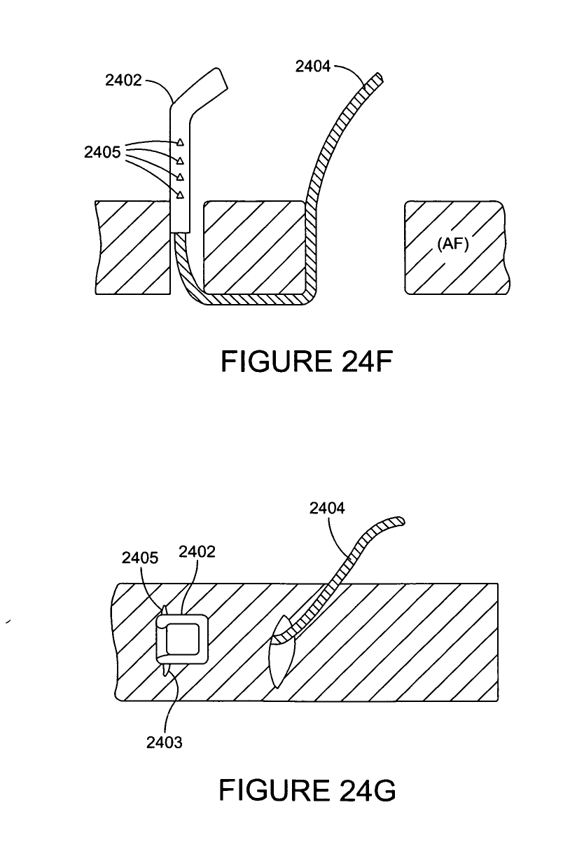

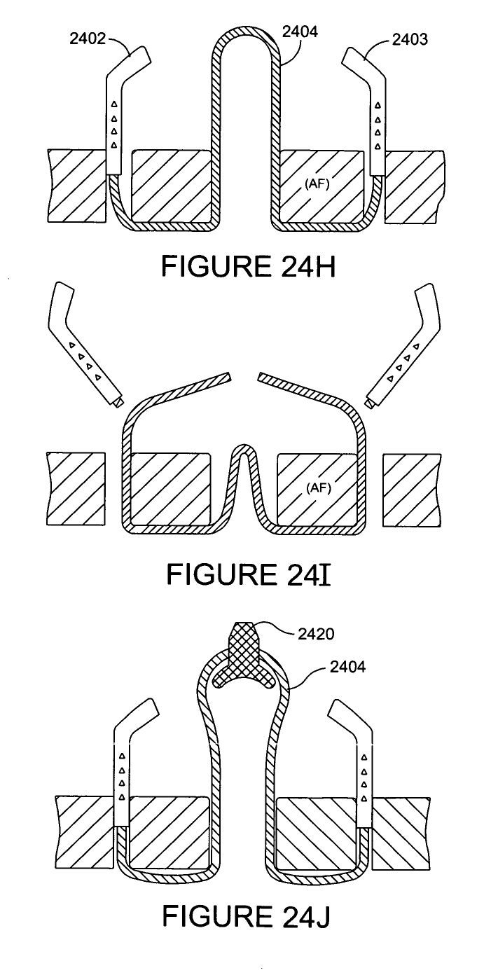

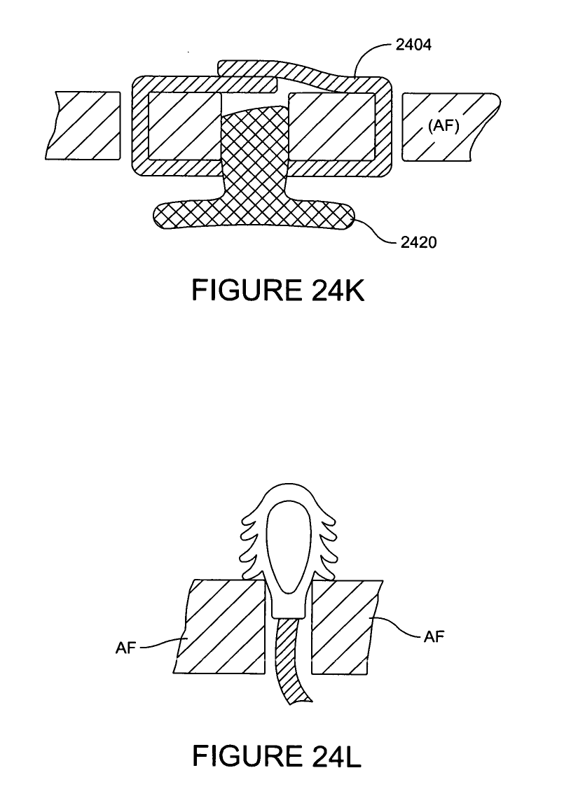

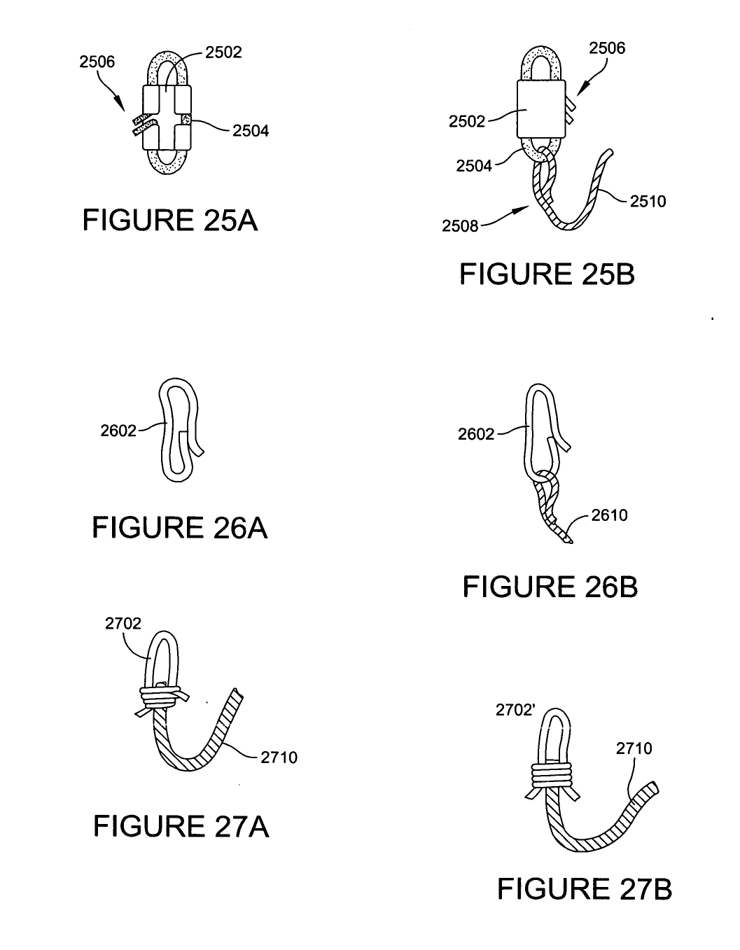

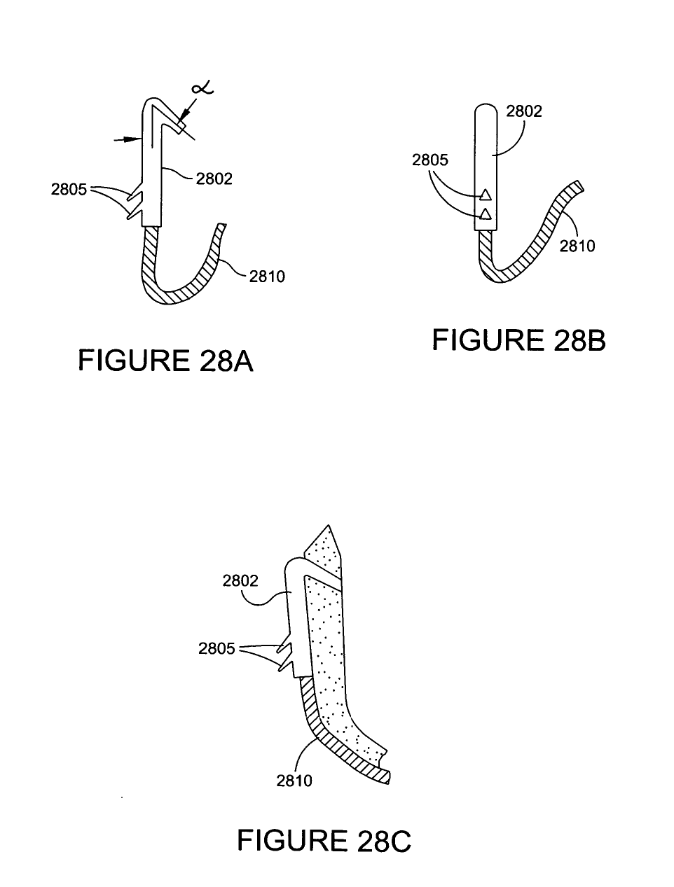

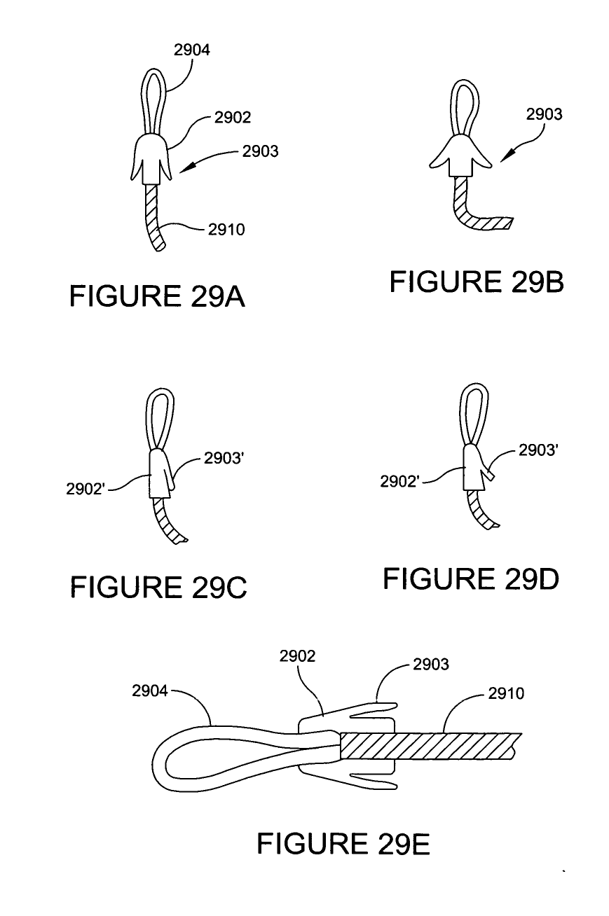

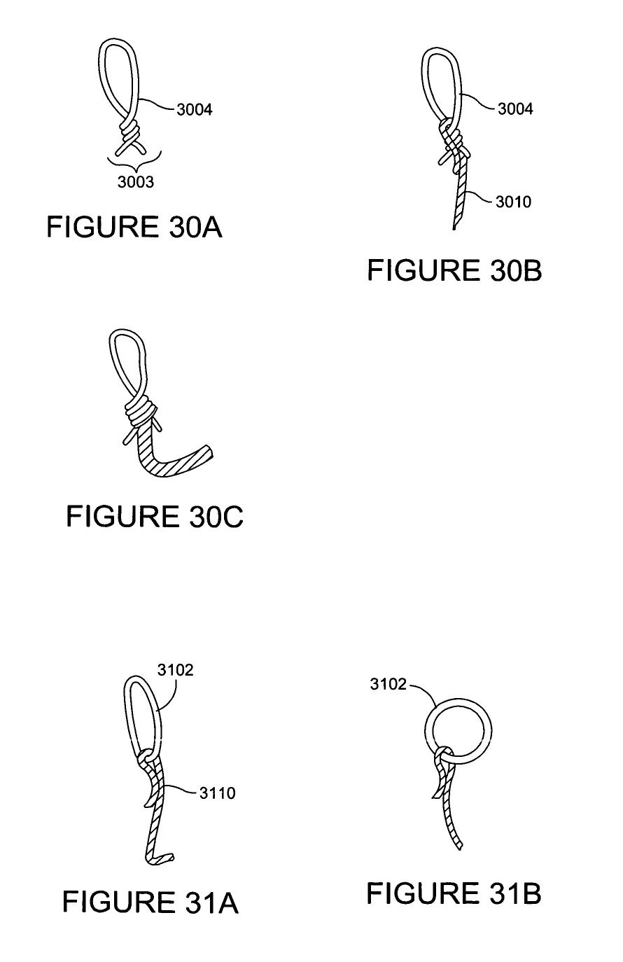

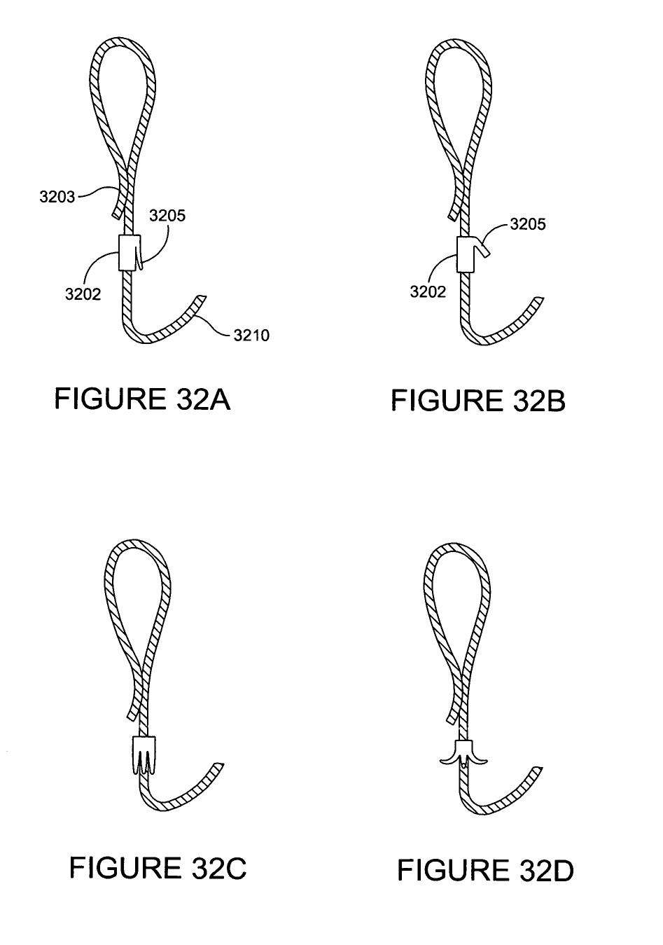

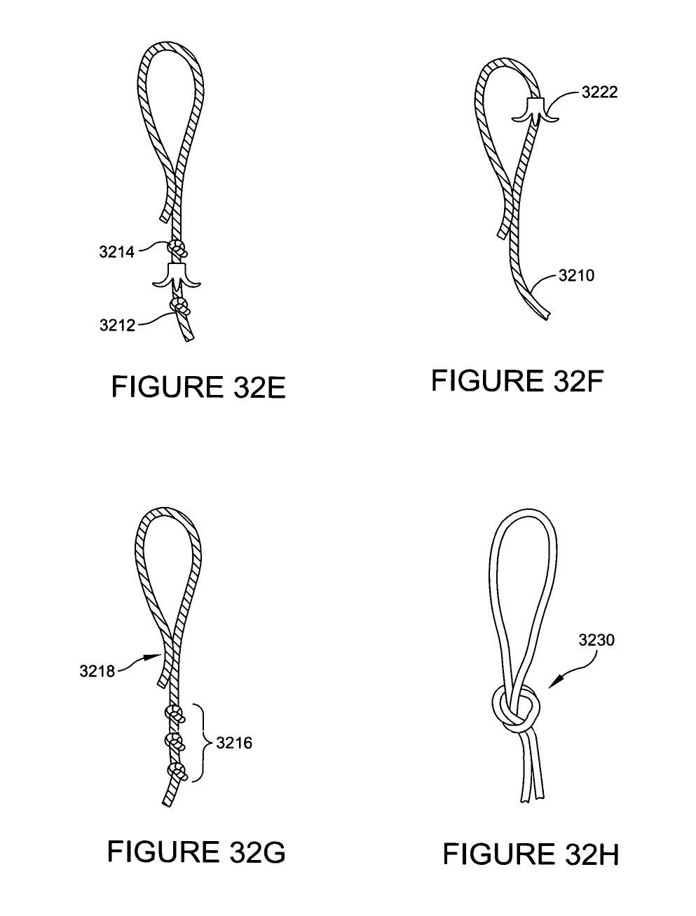

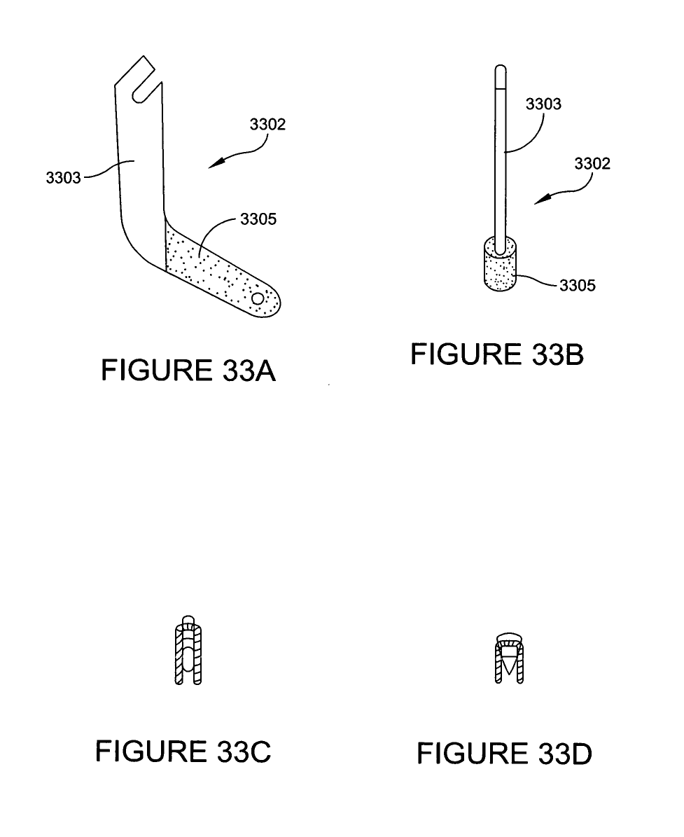

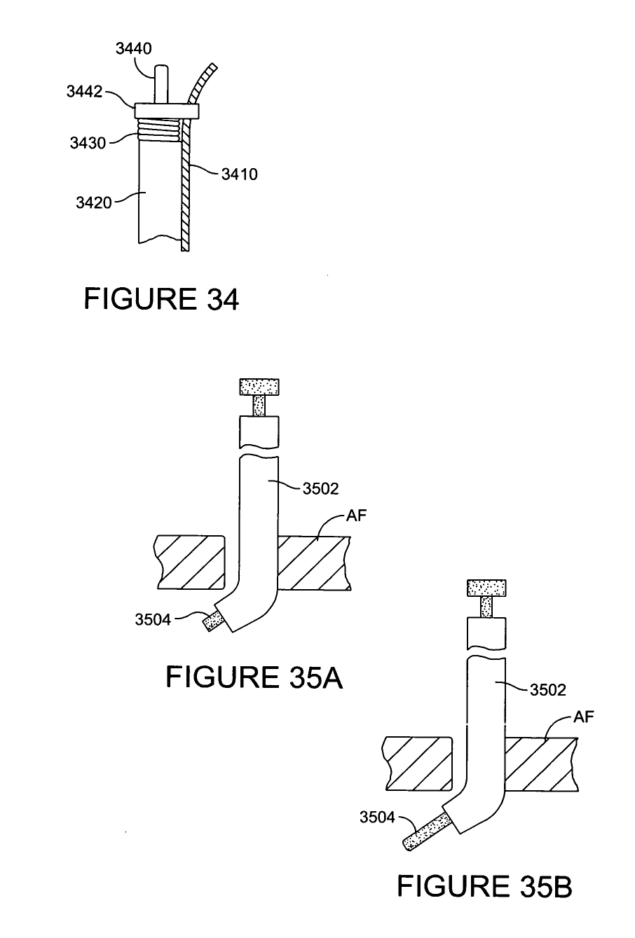

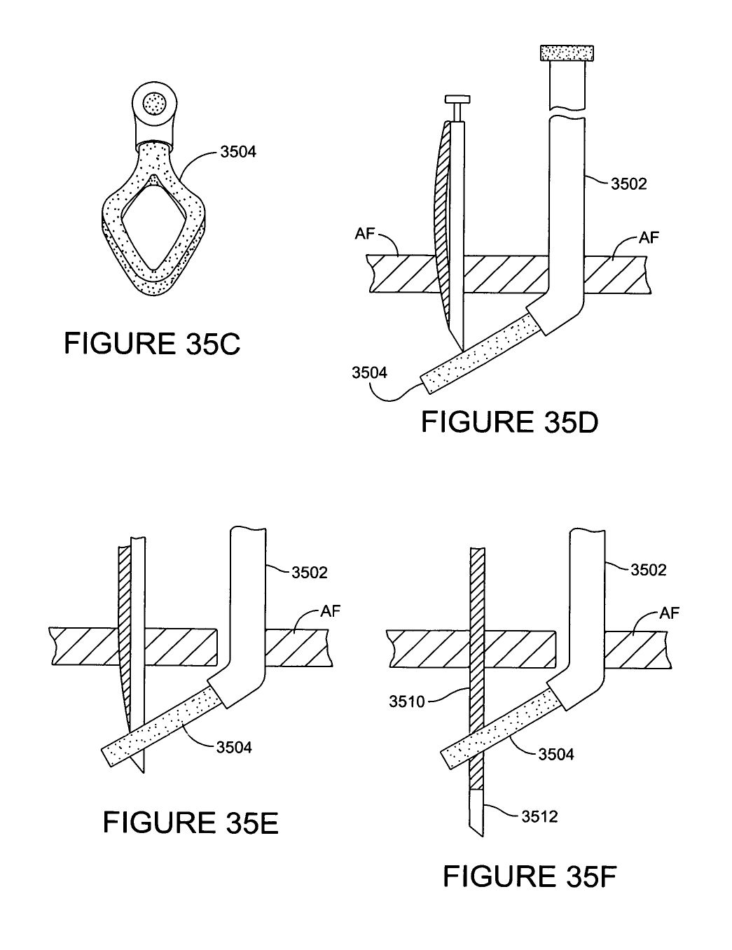

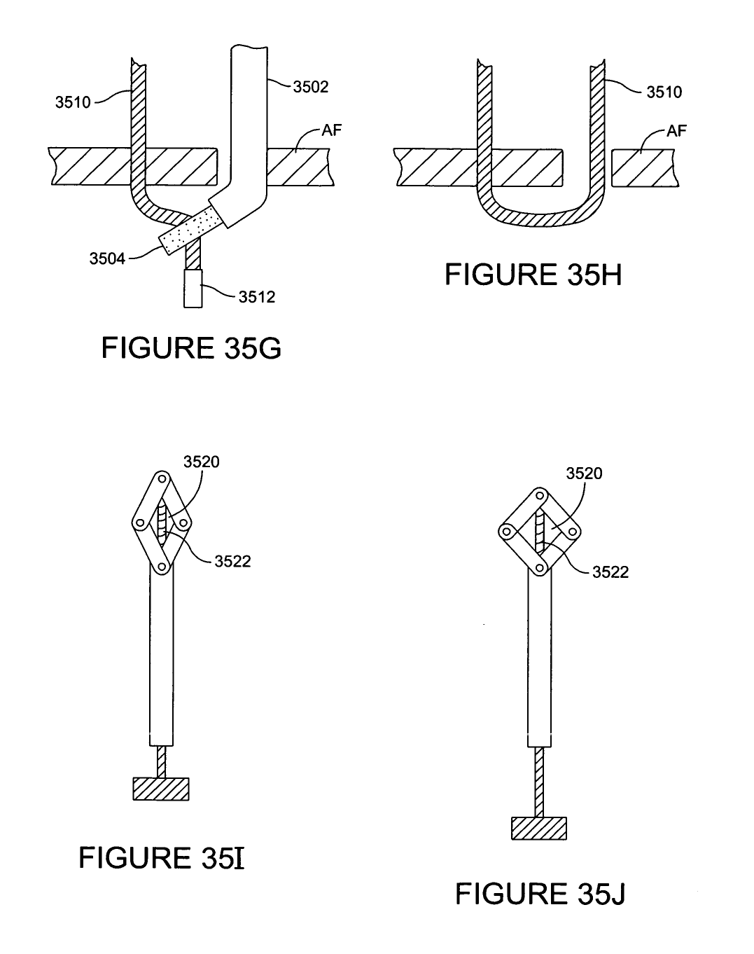

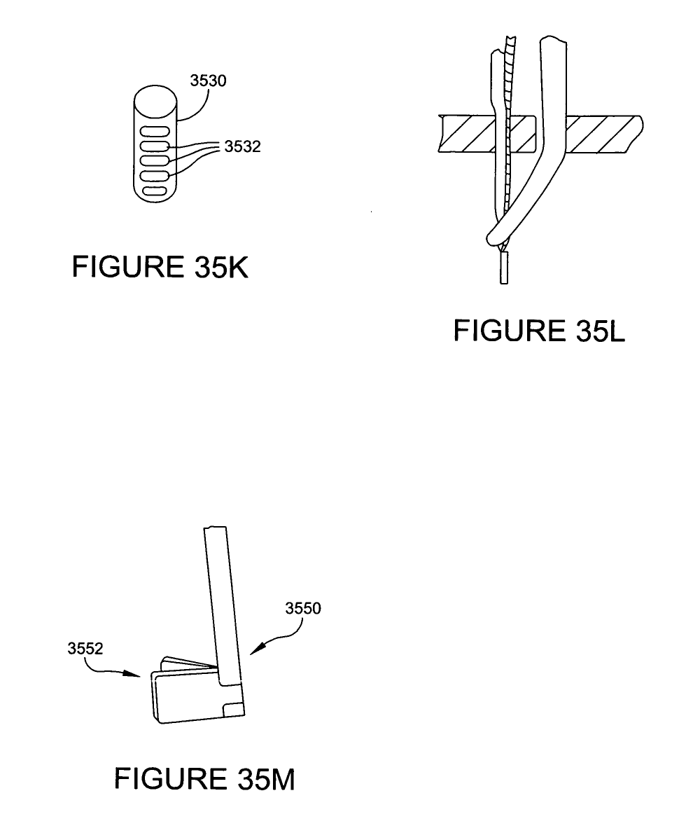

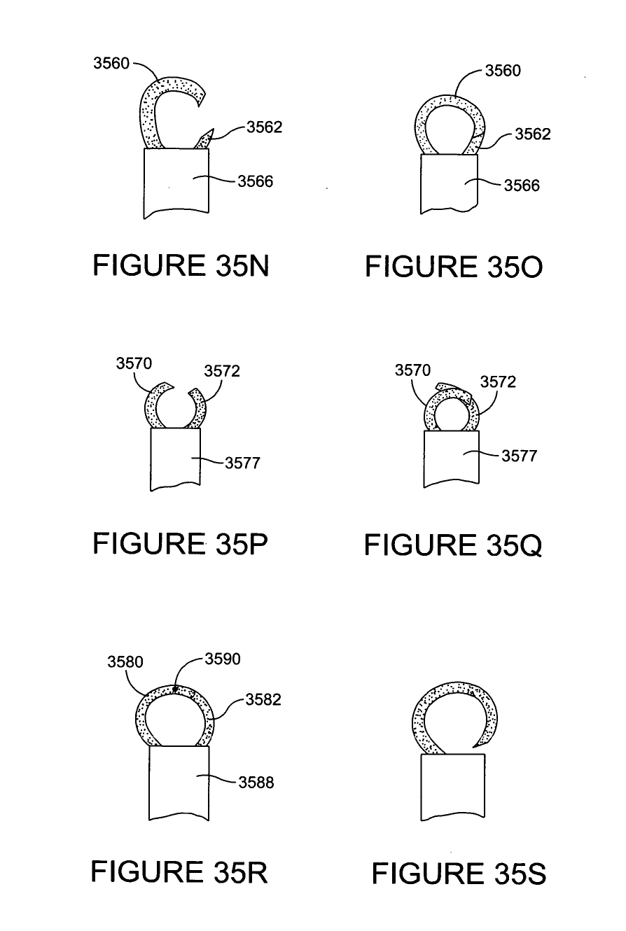

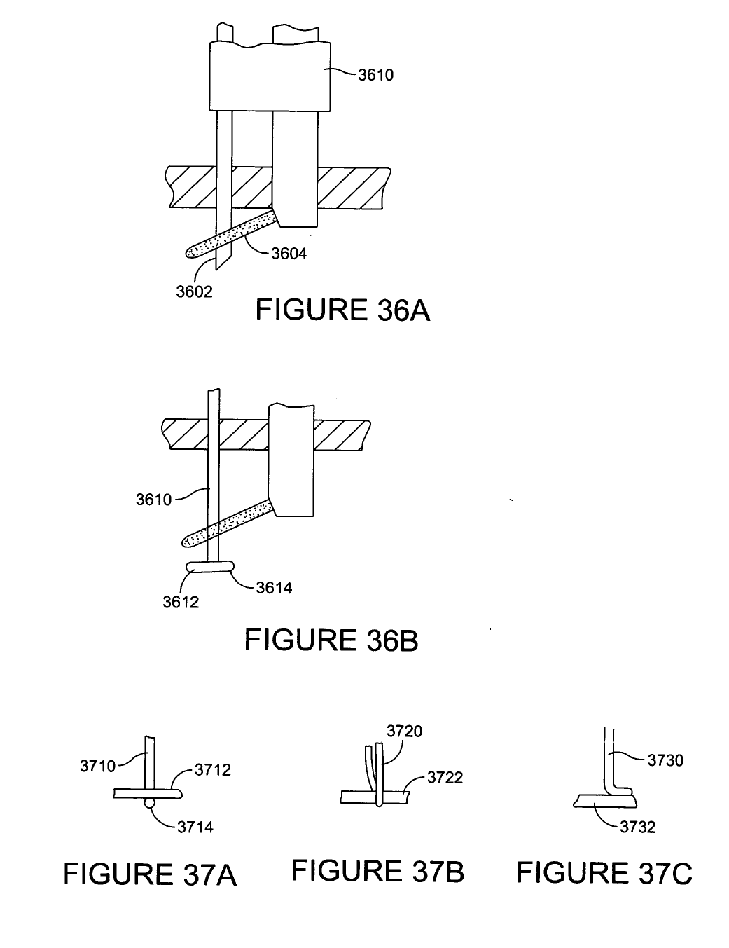

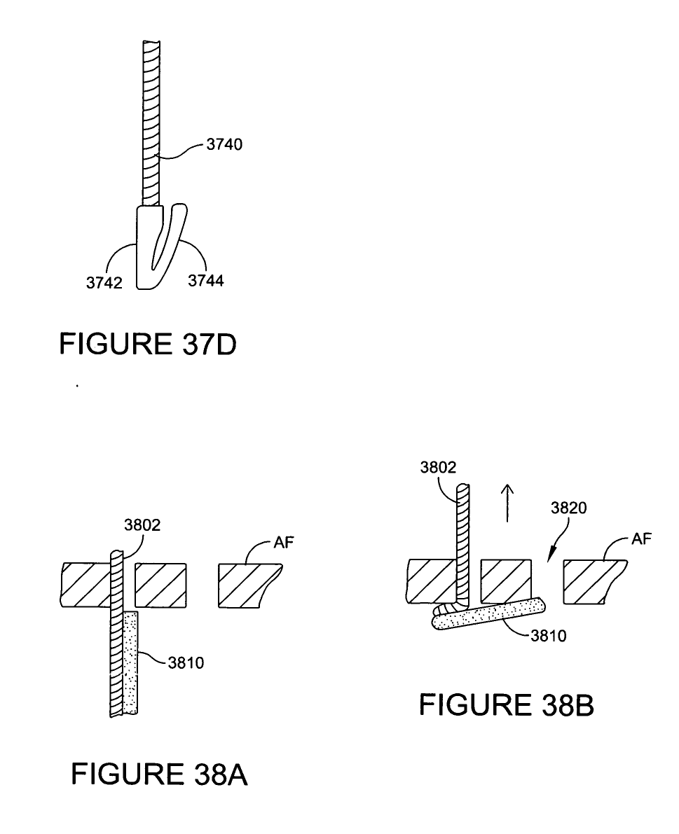

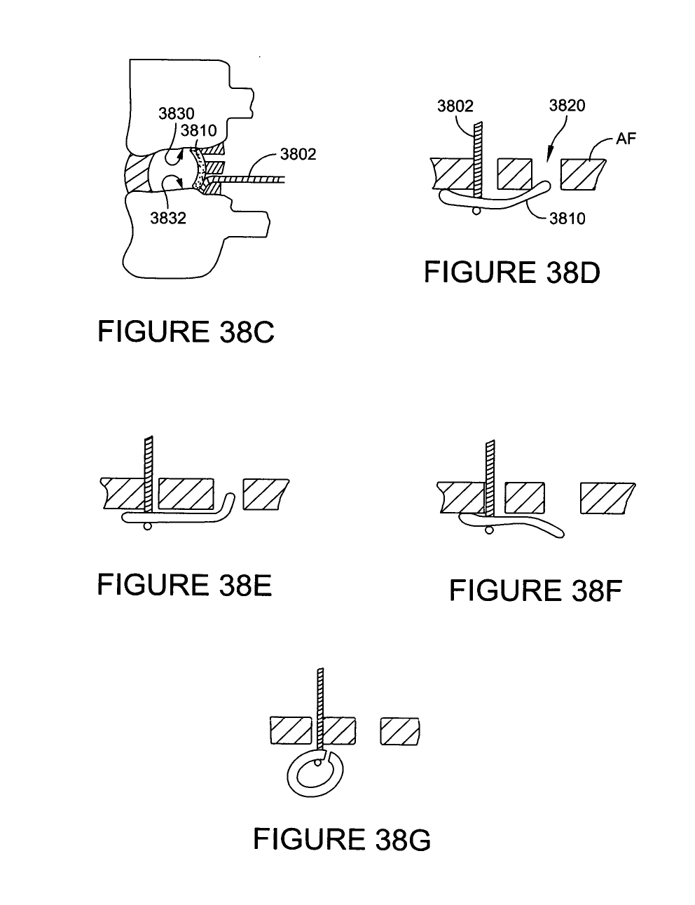

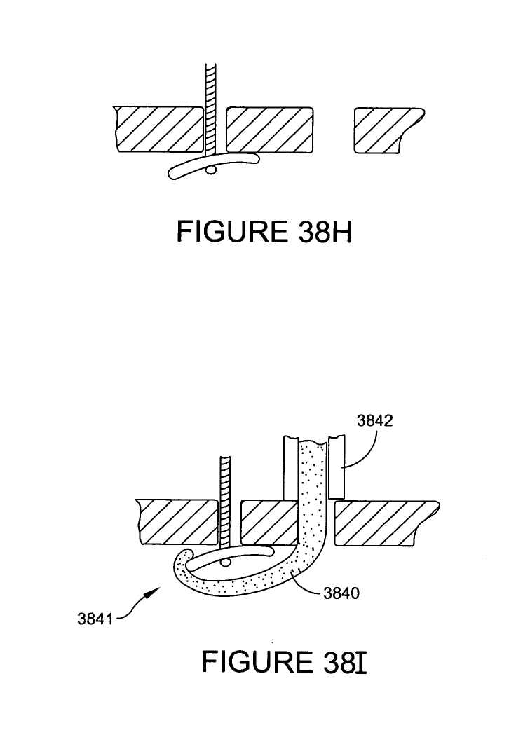

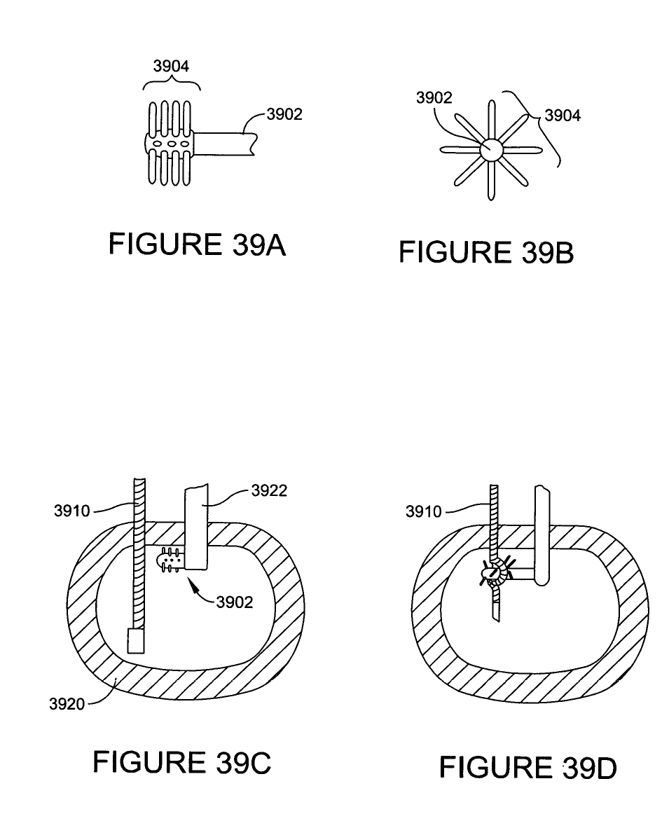

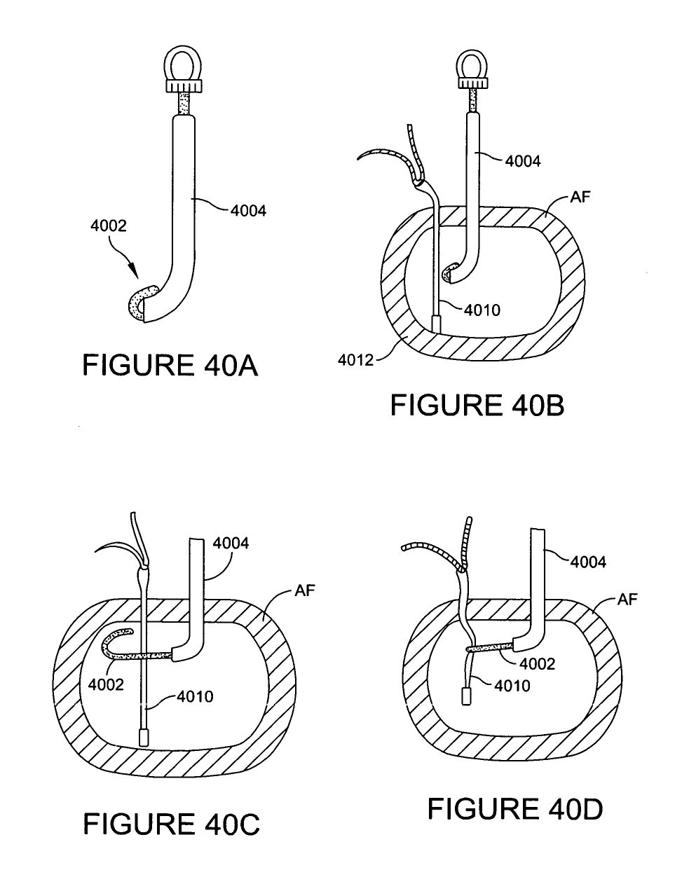

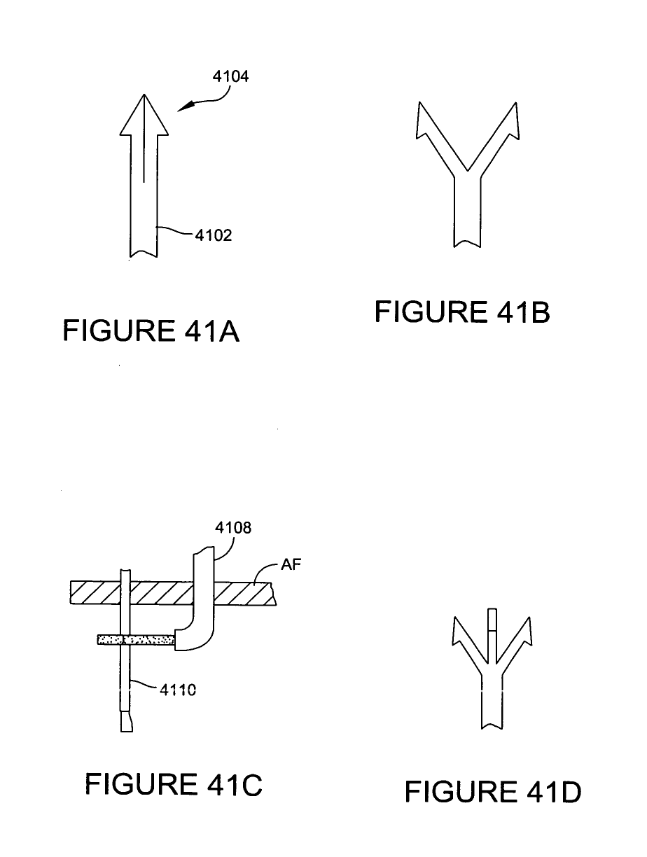

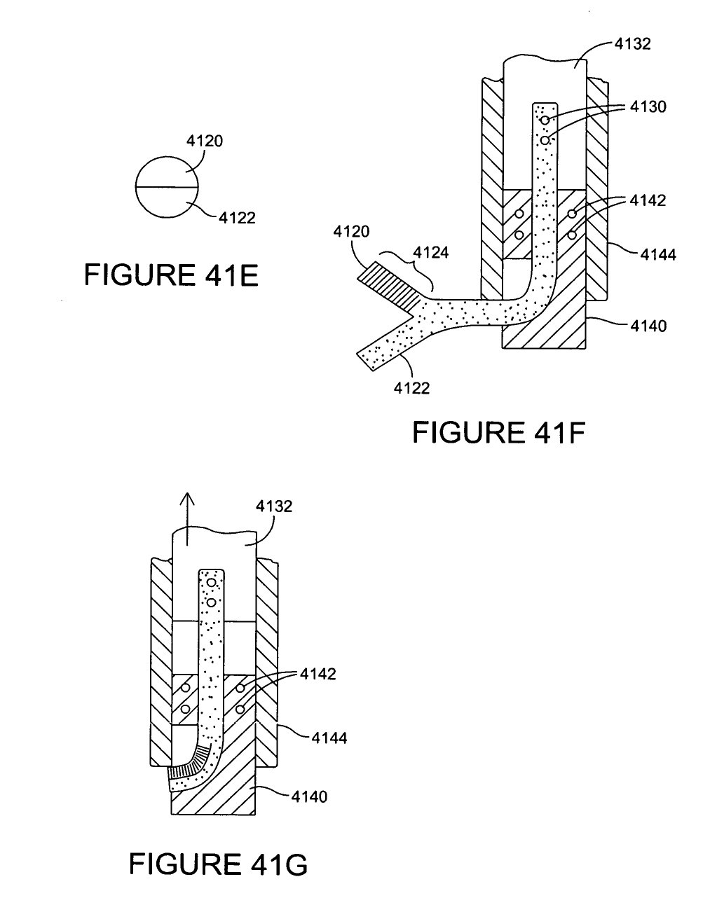

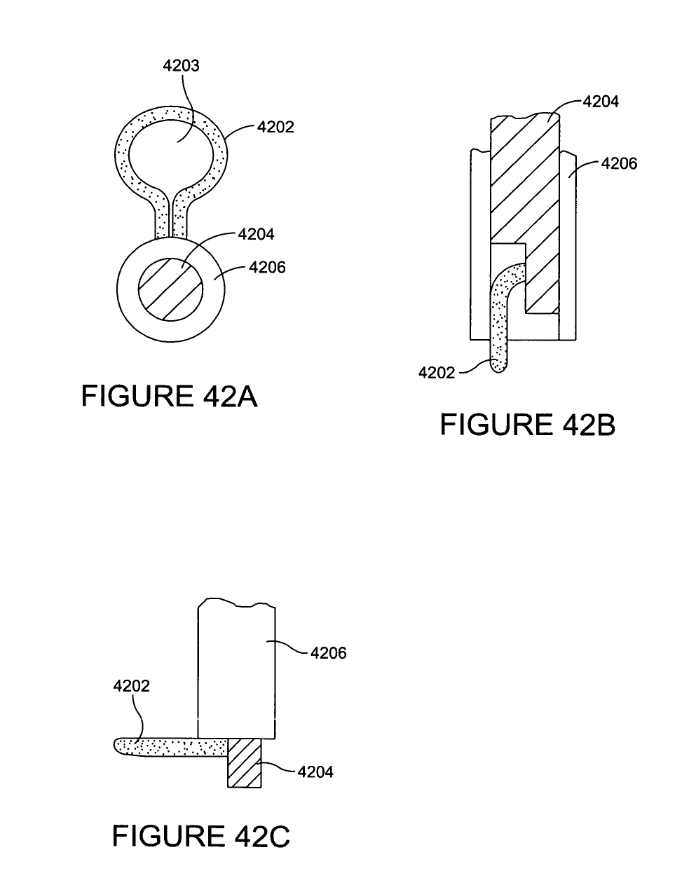

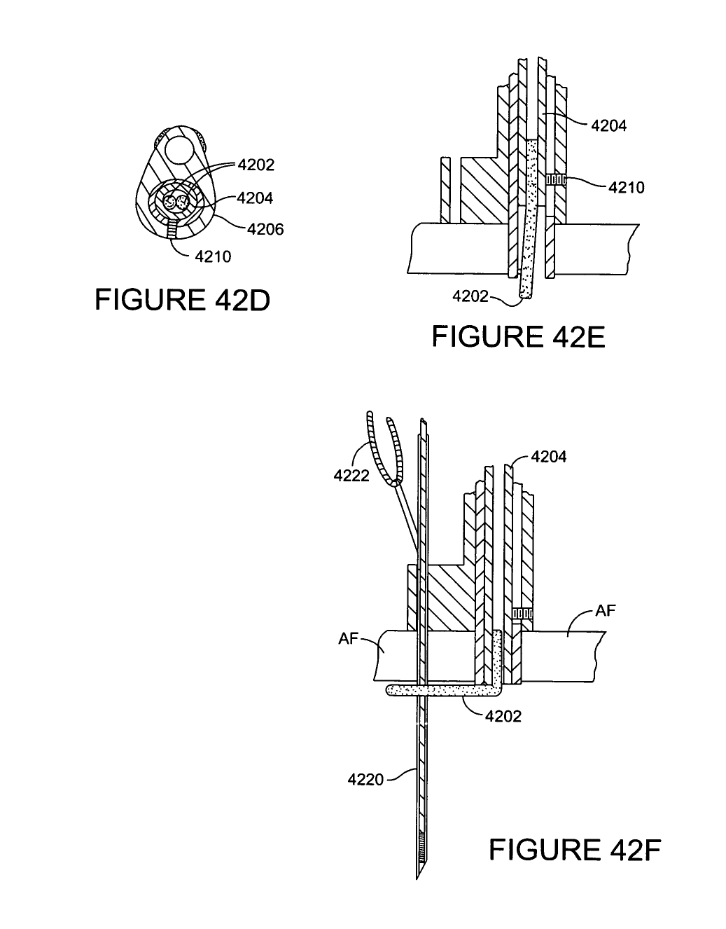

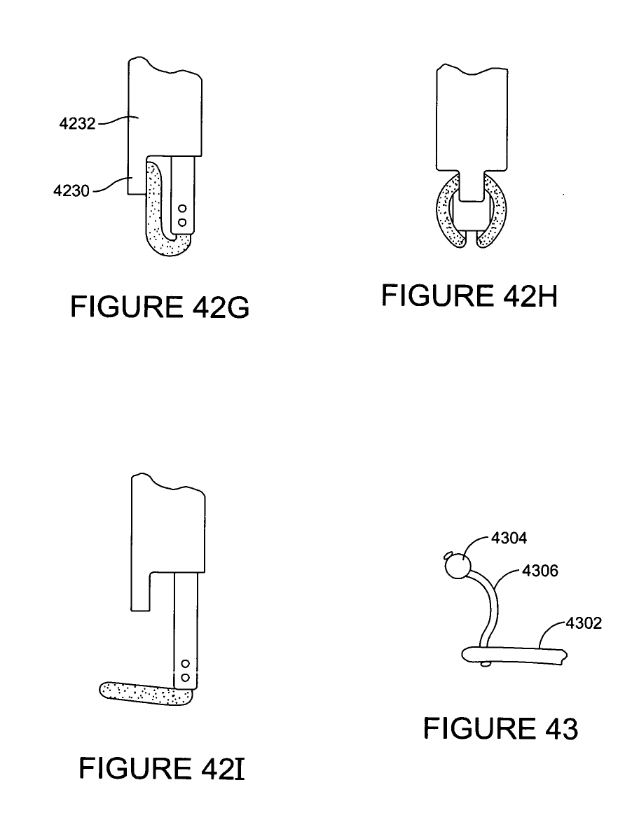

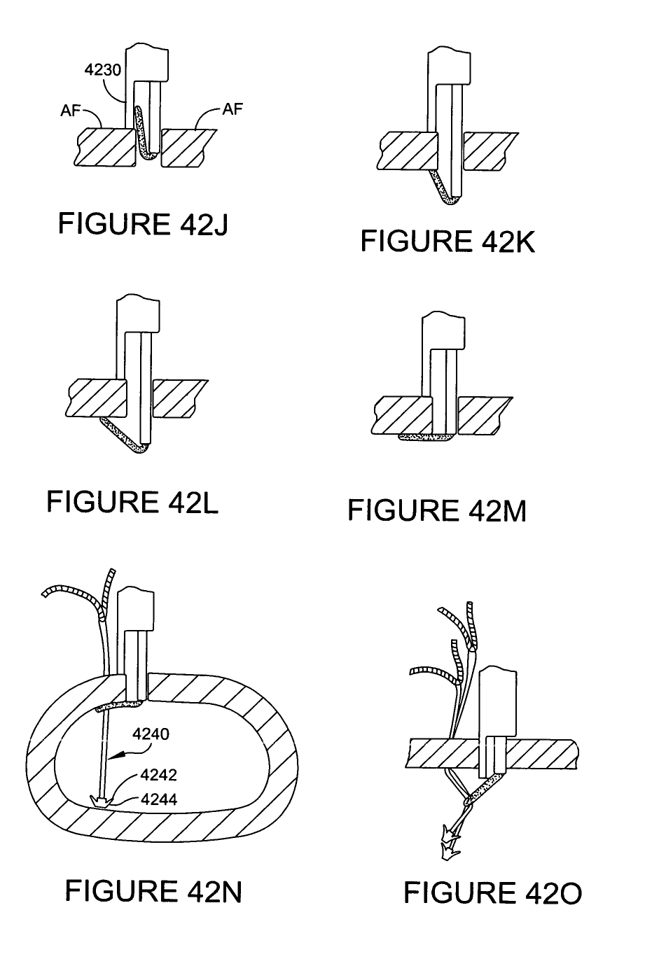

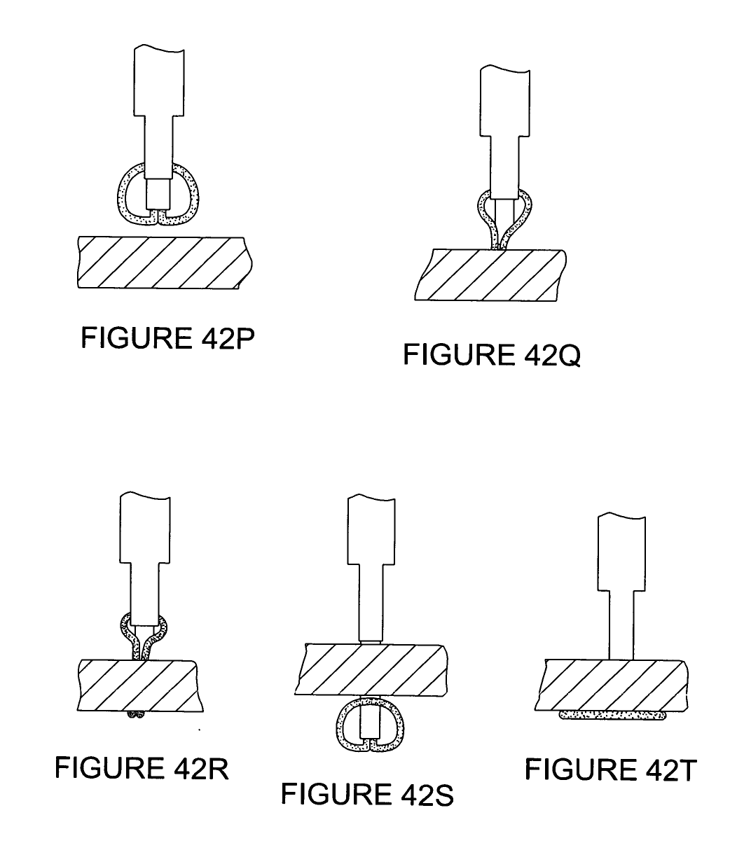

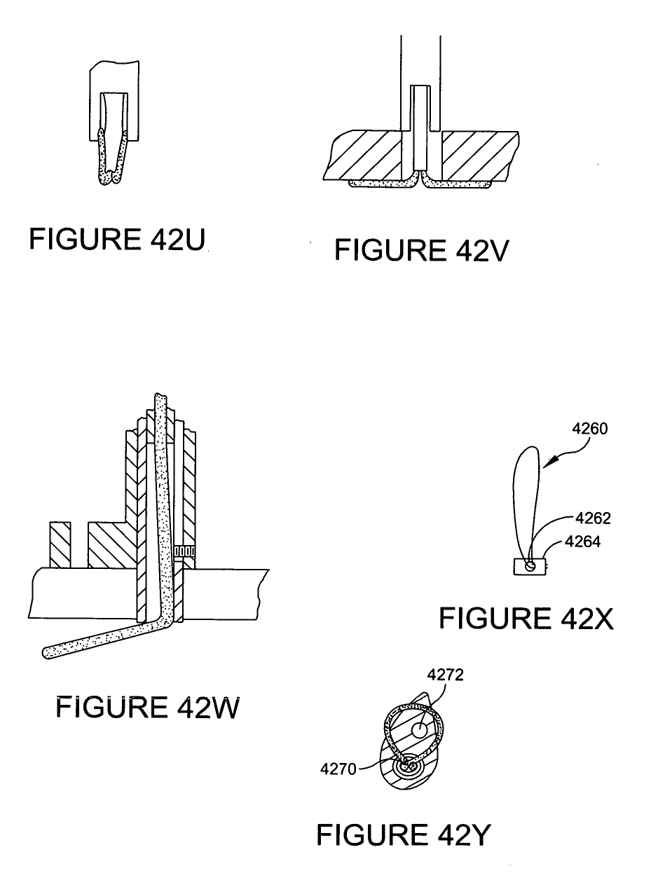

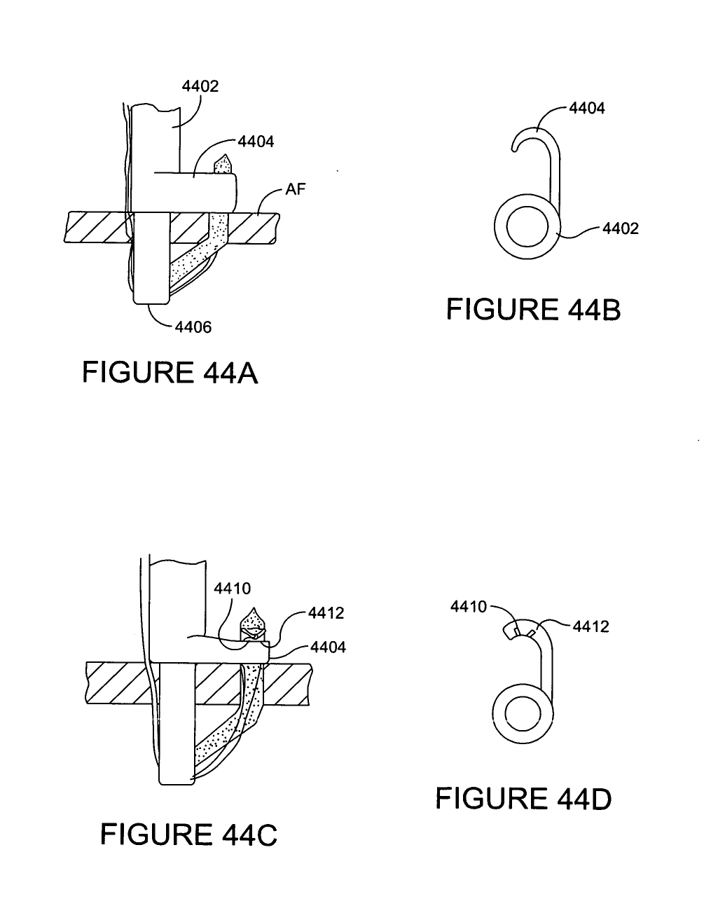

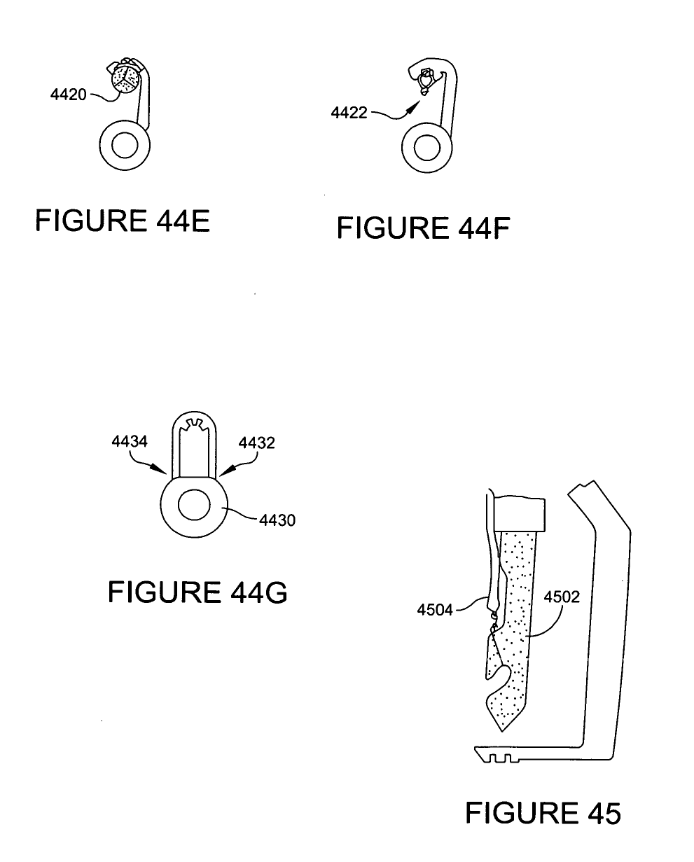

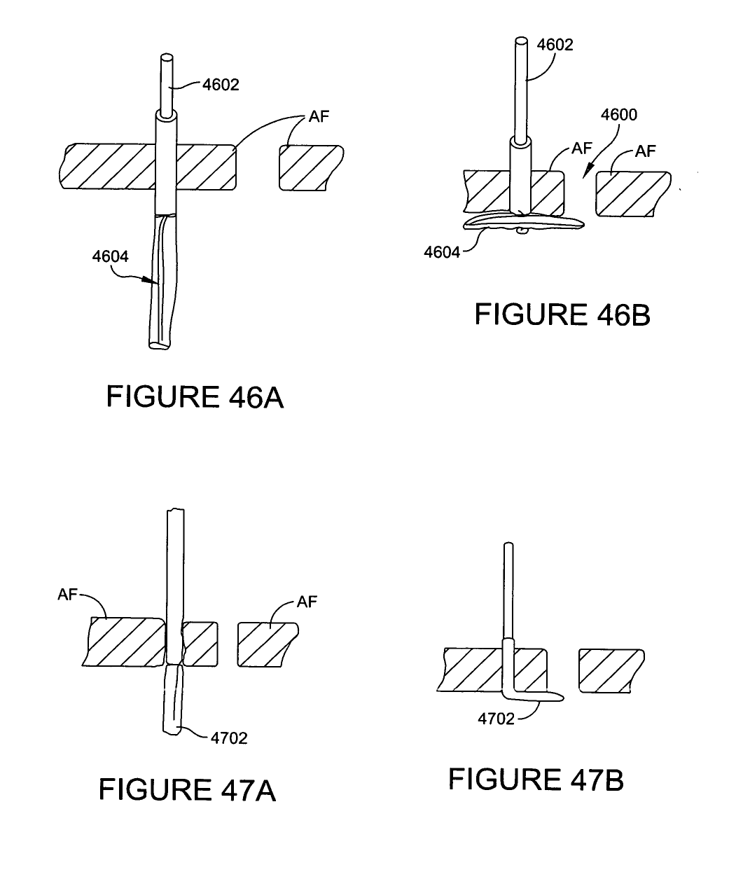

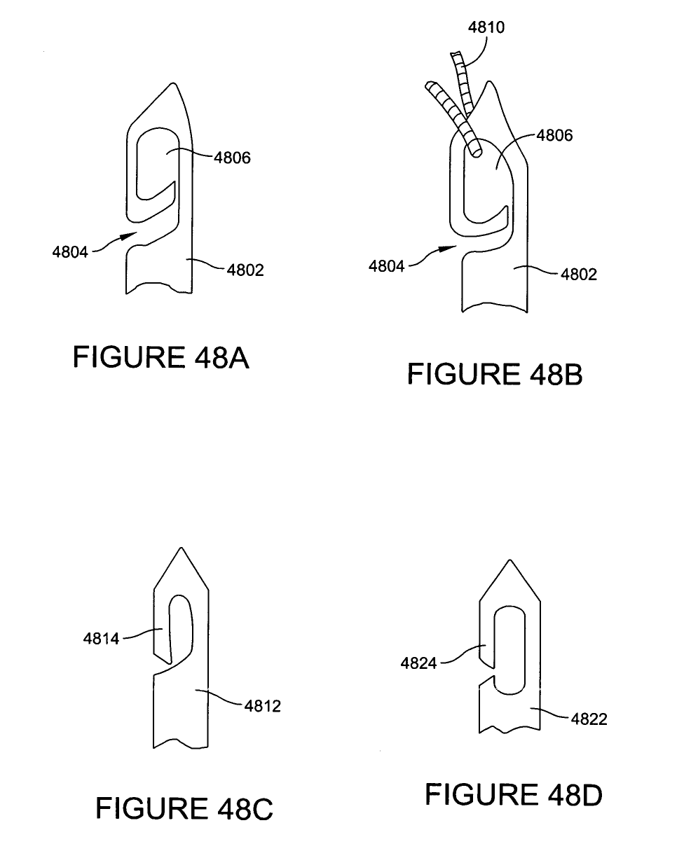

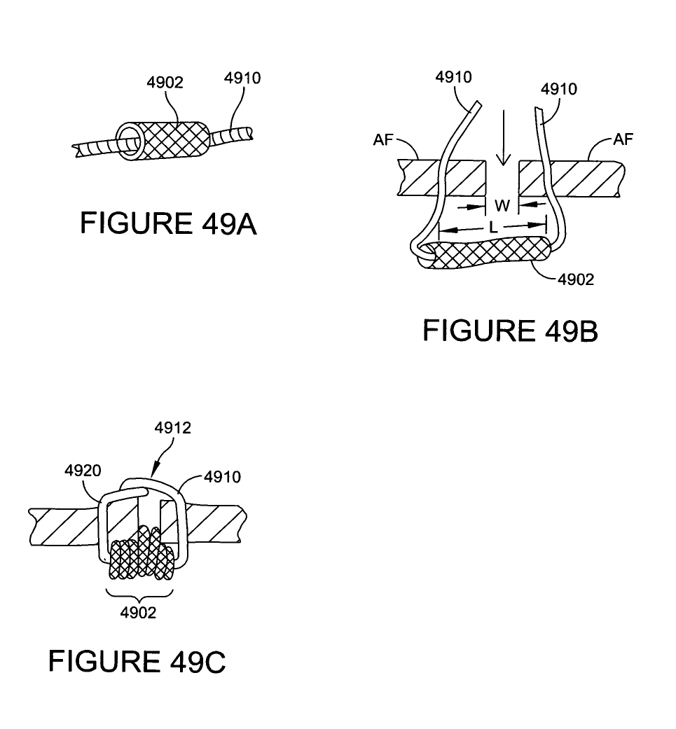

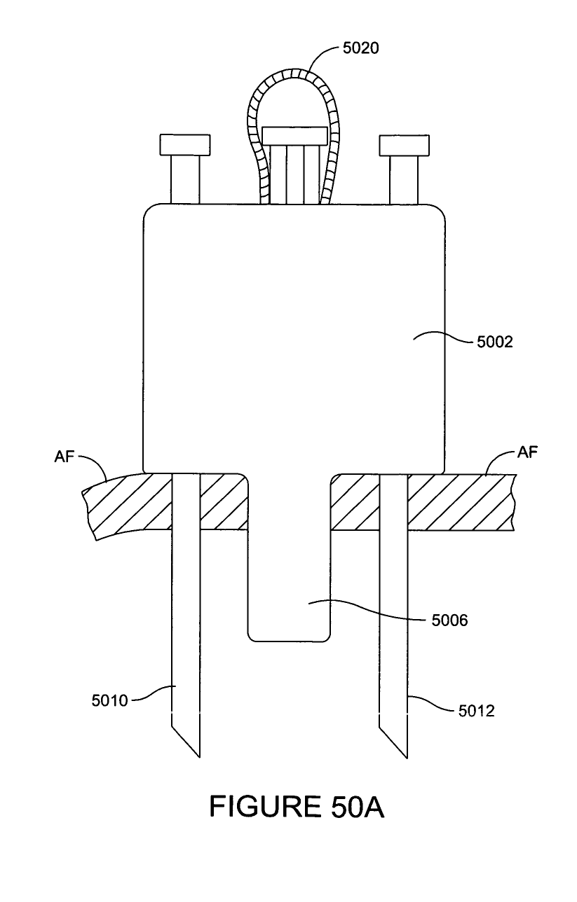

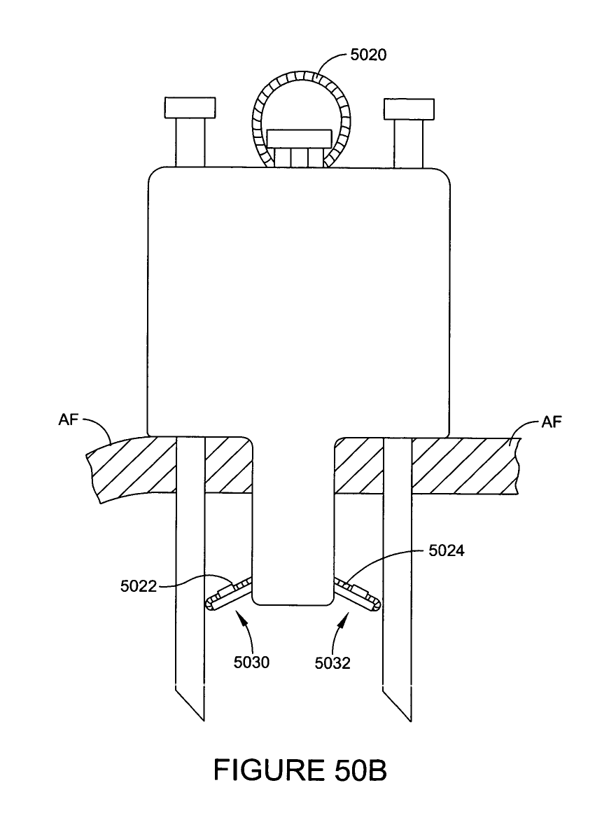

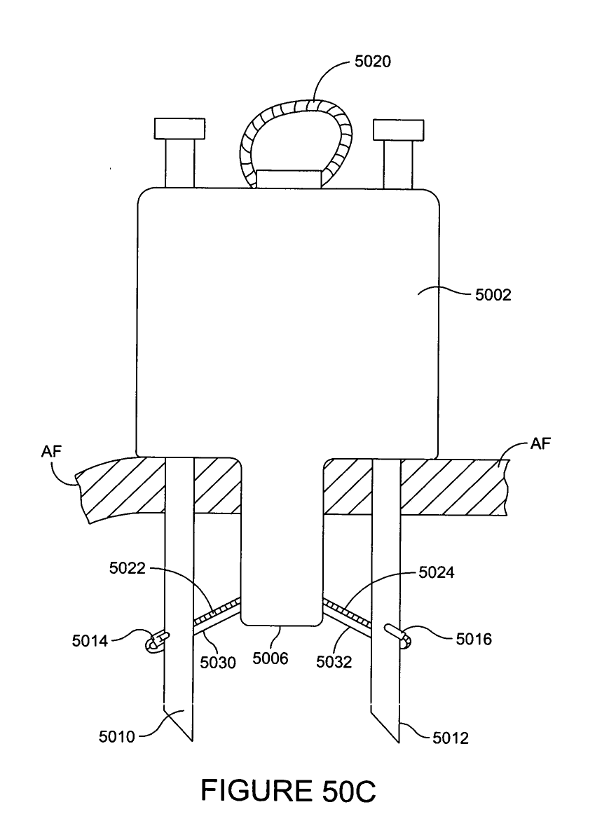

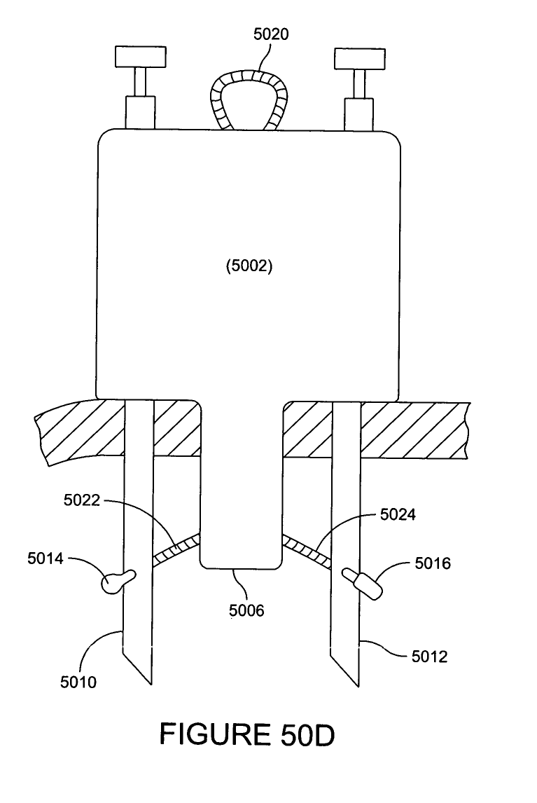

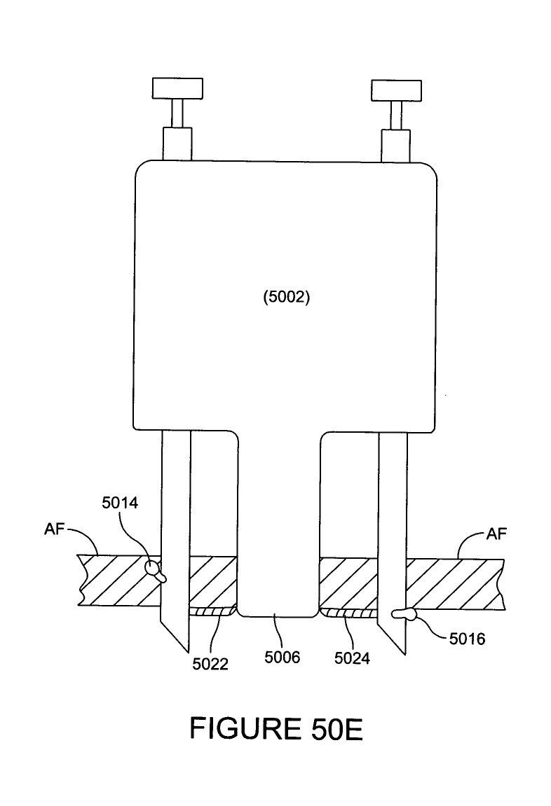

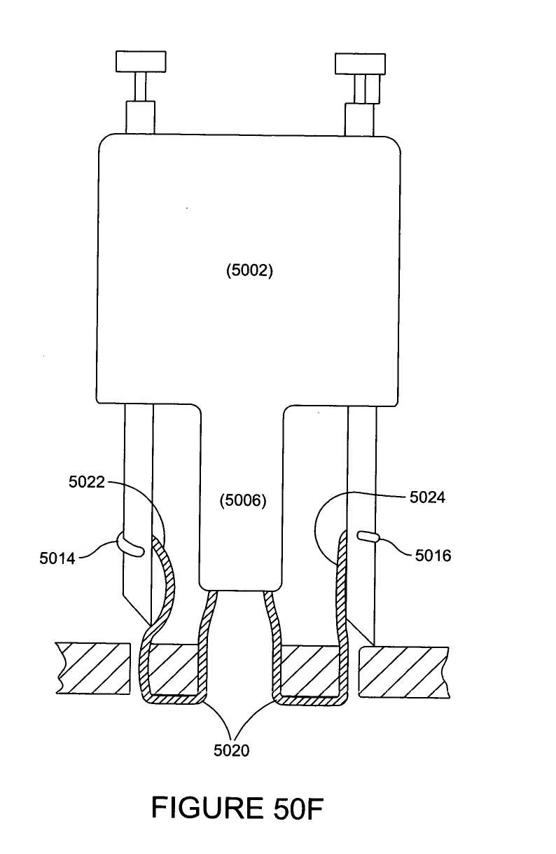

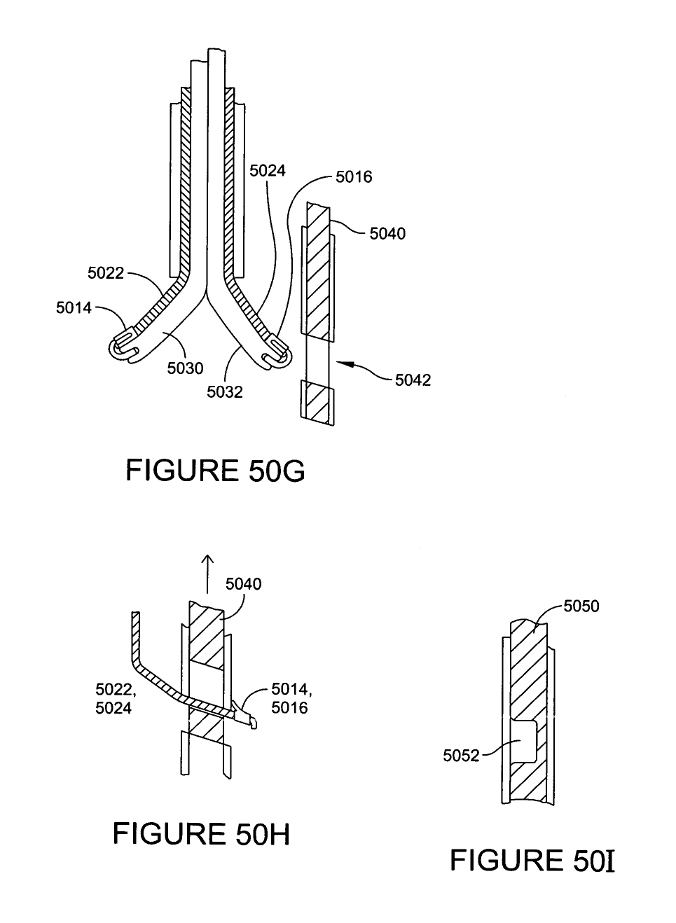

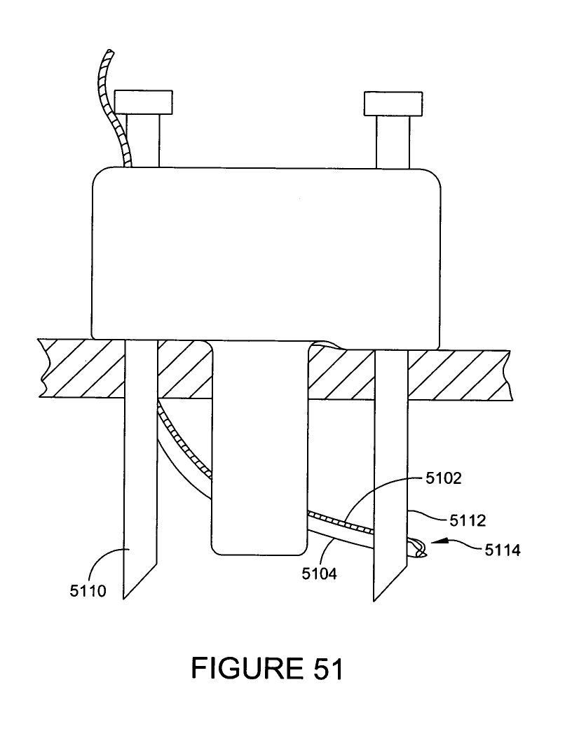

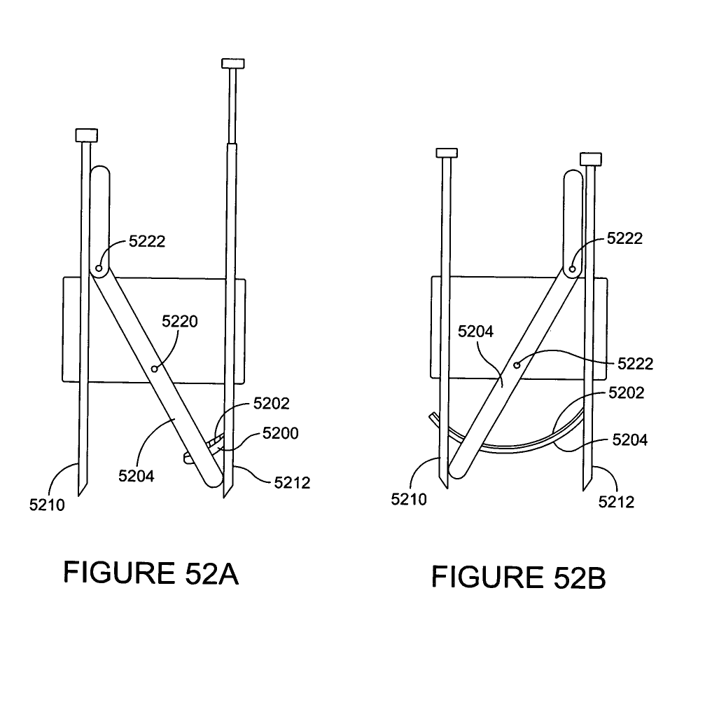

FIG. 5A is an oblique lateral view of an alternative embodiment of the invention drawn in FIG. 7;

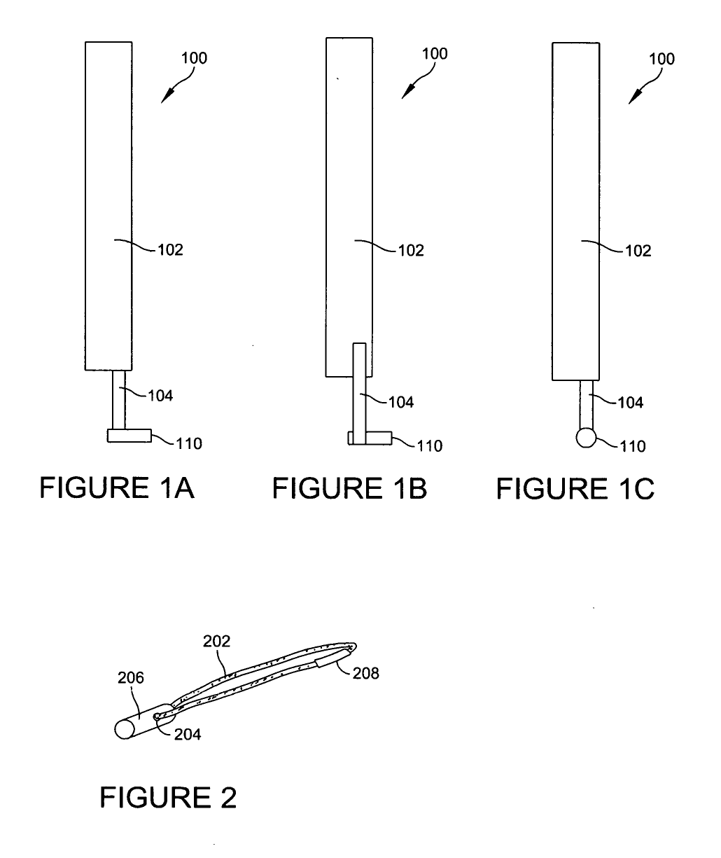

FIG. 8B is a lateral view of the embodiments of the invention drawn in FIGS. 1A and 8A and a superior view of an axial cross section of a portion of the AF;

FIG. 9A is a lateral view of an alternative embodiment of the invention drawn in FIG. 8A;

FIG. 9B is a lateral view of an alternative embodiment of the invention drawn in FIG. 9A;

FIG. 10A is a lateral view of an alternative embodiment of the invention drawn in FIG. 9A;

FIG. 10B is a lateral view of an alternative embodiment of the invention drawn in FIG. 10A;

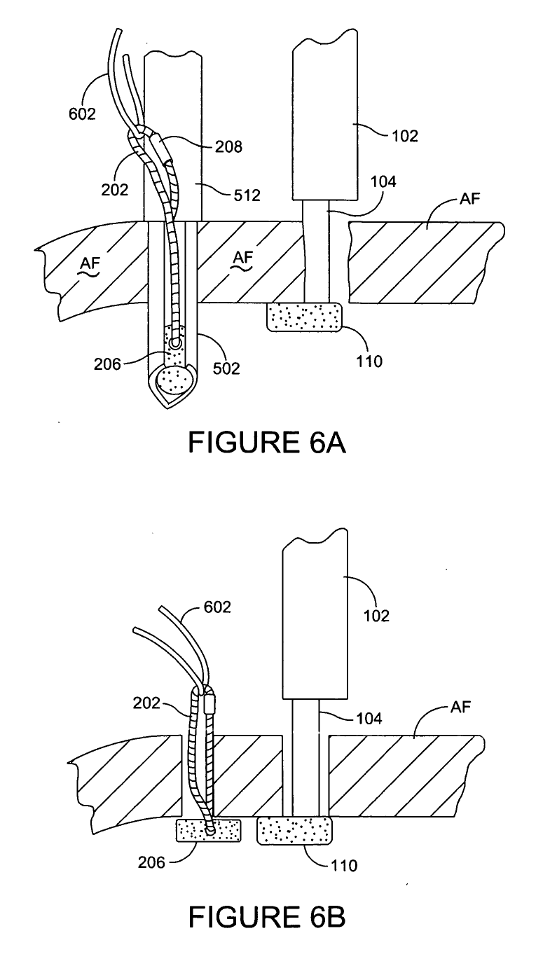

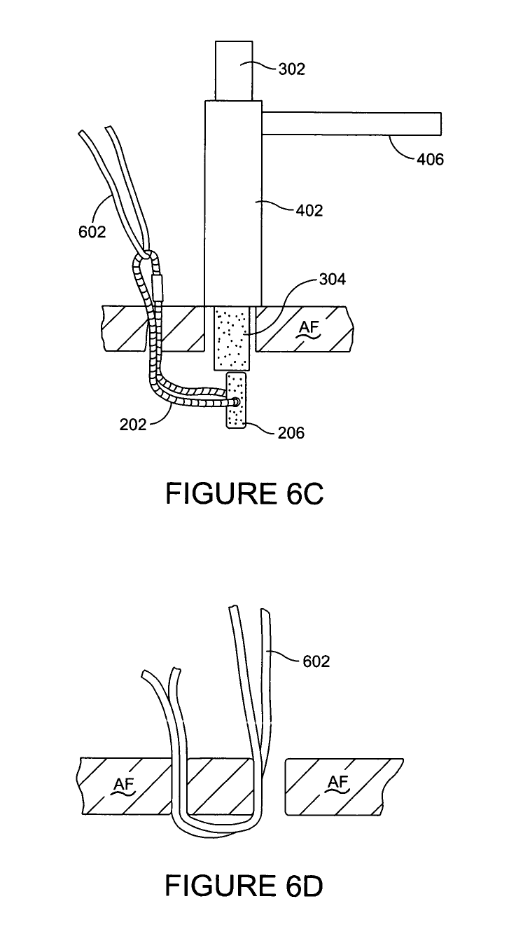

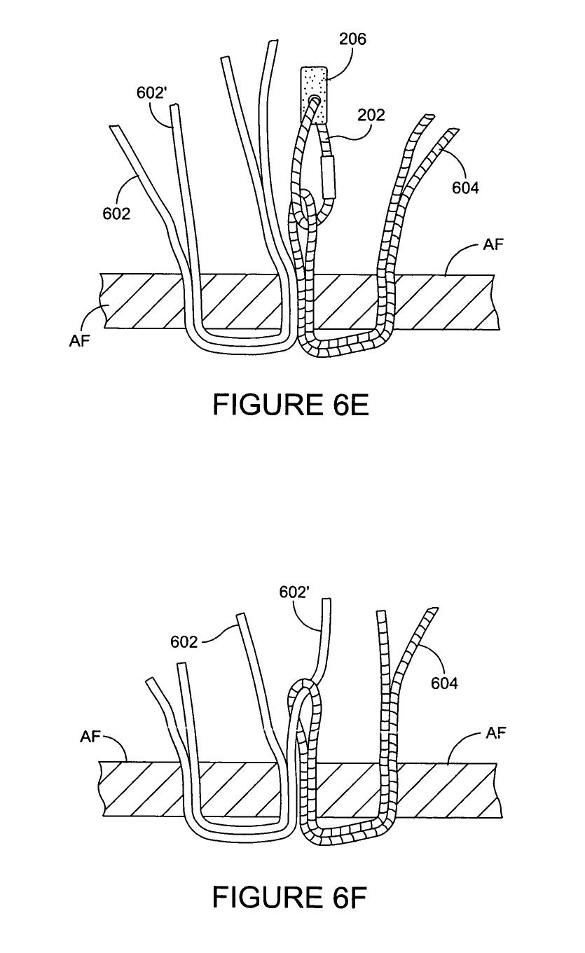

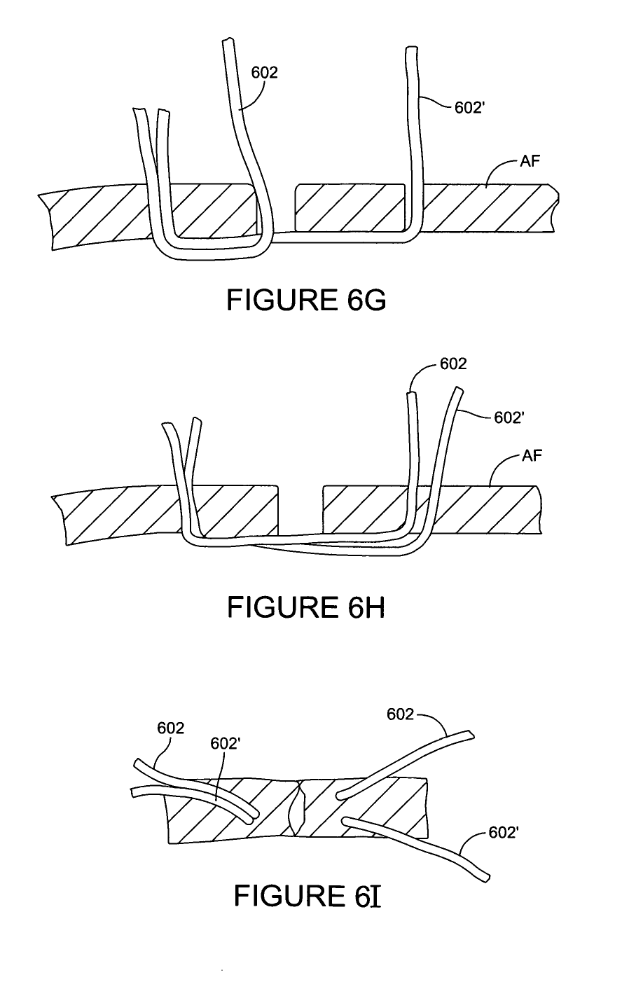

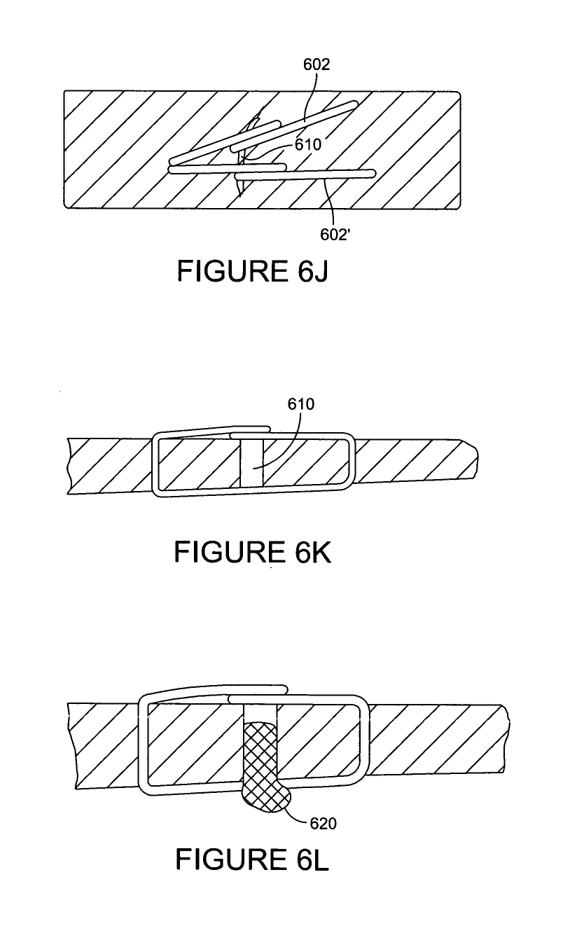

FIG. 11A is a posterior view of an alternative embodiment of the invention drawn in FIG. 6A and a coronal cross section of a portion of the spine;

FIG. 11B is a posterior view of an alternative embodiment of the invention drawn in FIG. 11A and a coronal cross section of a portion of the spine;

FIG. 11C is a posterior view of an alternative embodiment of the invention drawn in FIG. 11B and a coronal cross section of a portion of the spine;

FIG. 11D is a posterior view of an alternative embodiment of the invention drawn in FIG. 11C and a coronal cross section of a portion of the spine;

FIG. 11E is a posterior view of an alternative embodiment of the invention drawn in FIG. 11D and a coronal cross section of a portion of the spine;

FIG. 11F is a superior view of partial transverse cross section the embodiment of the invention drawn in FIG. 11E and a portion of the AF;

FIG. 11G is a lateral view of a partial sagittal cross section the embodiment of the invention drawn in FIG. 11G and a portion of the spine;

FIG. 12A is a lateral view of an alternative embodiment of the invention drawn in FIG. 2;

FIG. 12B is superior view of the embodiment of the invention drawn in FIG. 12A, an alternative embodiment of the invention drawn in FIG. 6C, and an axial cross section of the portion of the AF;

FIG. 12C is a superior view of the embodiment of the invention and portion of the AF drawn in FIG. 12B;

FIG. 12D is a superior view of the embodiment of the invention and portion of the AF drawn in FIG. 12C;

FIG. 12E is a superior view of the embodiment of the invention and portion of the AF drawn in FIG. 12D;

FIG. 12F is a superior view of the embodiment of the invention and portion of the AF drawn in FIG. 12E;

FIG. 12G is a superior view of the embodiment of the invention and portion of the AF drawn in FIG. 12F;

FIG. 12H is a superior view of the embodiment of the invention and portion of the AF drawn in FIG. 12G;

FIG. 13A is an oblique lateral view of an alternative embodiment of the invention drawn in FIG. 12A;

FIG. 13B is a superior view of the embodiment of the invention drawn in FIG. 13A and a portion of the AF;

FIG. 13C is a superior view of the embodiment of the invention drawn in FIG. 13B and a portion of the AF;

FIG. 13D is a superior view of the embodiment of the invention drawn in FIG. 13C and a portion of the AF;

FIG. 13E is a superior view of the embodiment of the invention drawn in FIG. 13D and a portion of the AF;

FIG. 14 is a superior view of an alternative embodiment of the invention drawn in FIG. 13A;

FIG. 15A is a superior view of an alternative embodiment of the invention drawn in FIG. 14;

FIG. 15B is anterior view of the embodiment of the invention drawn in FIG. 15A;

FIG. 16A is an oblique lateral view of an alternative embodiment of the invention drawn in FIG. 15A;

FIG. 16B is an oblique lateral view of an alternative embodiment of the invention drawn in FIG. 16A;

FIG. 16C is an oblique lateral view of an alternative embodiment of the invention drawn in FIG. 16B;

FIG. 17 is an oblique lateral view of an alternative embodiment of the invention drawn in FIG. 16C;

FIG. 18 is a lateral view of an alternative embodiment of the invention drawn in FIG. 17;

FIG. 19 is a superior view of an alternative embodiment of the invention drawn in FIG. 18;

FIG. 20 is a superior view of an alternative embodiment of the invention drawn in FIG. 19;

FIG. 21A is a superior view of a partial axial cross section an alternative embodiment of the invention drawn FIG. 6C and a portion of the AF;

FIG. 21B is a superior view of a partial axial cross section of the embodiment of the invention drawn in FIG. 21A and a portion of the AF;

FIG. 21C is a superior view of a partial axial cross section of the embodiment of the invention drawn in FIG. 21B and a portion of the AF;

FIG. 22A is a lateral view of the distal end of an alternative embodiment of the invention drawn in FIG. 5A;

FIG. 22B is a lateral view of a longitudinal cross section of distal end of the embodiment of the invention drawn in FIG. 22A;

FIG. 23A is a lateral view of the first end of an alternative embodiment of the invention drawn in FIG. 20;

FIG. 23B is a superior view of the embodiments of the invention drawn in FIGS. 22A & 23A and a partial axial cross section of a portion of the AF;

FIG. 23C is a superior view of the embodiment of the invention and portion of the AF drawn in FIG. 23B;

FIG. 23D is a superior view of a partially exploded embodiment of the invention and portion of the AF drawn in FIG. 23C;

FIG. 23E is a lateral view of the embodiment of the invention drawn in FIG. 23A;

FIG. 23F is a posterior view of the embodiment of the invention drawn in FIG. 23E and an IVD;

FIG. 23G is a posterior view of the embodiment of the invention and IVD drawn in FIG. 23F;

FIG. 24A is a lateral view of an alternative embodiment of the invention drawn in FIG. 23E;

FIG. 24B is a lateral view of a partial longitudinal cross section of the distal end of an alternative embodiment of the invention drawn in FIG. 23B and a first end of the embodiment of the invention drawn in FIG. 24A;

FIG. 24C is a lateral view of the distal end of the embodiment of the invention drawn in FIG. 24B;

FIG. 24D is an anterior view of the distal end of the embodiment of the invention drawn in FIG. 24C;

FIG. 24E is a superior view of the distal end of the embodiment of the invention drawn in FIG. 24D and a partial axial cross section of a portion of the AF;

FIG. 24F is a superior view of the first end and the portion of the AF drawn in FIG. 24E;

FIG. 24G is a posterior view of the embodiment of the invention and IVD drawn in FIG. 24F;

FIG. 24H is a superior view of the embodiment of the invention drawn in FIG. 24G and an axial view of a portion of the AF;

FIG. 24I is a superior view of an exploded embodiment and portion of the AF drawn in FIG. 24H;

FIG. 24J is a superior view of an alternative embodiment of the invention drawn in FIG. 24I and an axial cross section of a portion of the AF;

FIG. 24K is a superior view of the embodiment of the invention and portion of the AF drawn in FIG. 24J;

FIG. 24L is a superior view of a first end of the embodiment of the invention drawn in FIG. 24J and an axial cross section of a portion of the AF;

FIG. 25A is an inferior view of a portion of an alternative embodiment of the invention drawn in FIG. 24L;

FIG. 25B is a superior view of the embodiment of the invention drawn in FIG. 25A and a portion of a flexible longitudinal fixation component;

FIG. 26A is a superior view of an alternative embodiment of the invention drawn in FIG. 25A;

FIG. 26B is a superior view of the embodiment of the invention drawn in FIG. 26A and a portion of a flexible longitudinal fixation component;

FIG. 27A is a superior view of one end of an alternative embodiment of the invention drawn in FIG. 26B;

FIG. 27B is a superior view of one end of an alternative embodiment of the invention drawn in FIG. 27A;

FIG. 28A is a superior view of one end of an alternative embodiment of the invention drawn in FIG. 27B;

FIG. 28B is a lateral view of the embodiment of one end of the embodiment of the invention drawn in FIG. 28A;

FIG. 28C is a superior view of a partial longitudinal cross section of a portion of an alternative embodiment of the invention drawn in 24E and the embodiment of the invention drawn in FIG. 28A;

FIG. 29A is a superior view of one end of an alternative embodiment of the invention drawn in FIG. 28A;

FIG. 29B is a superior view of the end of the embodiment of the invention drawn in FIG. 29A;

FIG. 29C is a superior view of one end of an alternative embodiment of the invention drawn in FIG. 29A;

FIG. 29D is a superior view of the end of the embodiment of the invention drawn in FIG. 29C;

FIG. 29E is a superior view of a longitudinal cross section of the end of the embodiment of the invention drawn in FIG. 29B;

FIG. 30A is a superior view of an alternative embodiment of the invention drawn in FIG. 25A;

FIG. 30B is a superior view of the embodiment of the invention drawn in FIG. 30A and a portion of a flexible longitudinal fixation component;

FIG. 30C is a superior view of an alternative embodiment of the invention drawn in FIG. 30B;

FIG. 31A is a superior view of an alternative embodiment of the invention drawn in FIG. 30B;

FIG. 31B is a superior view of the embodiment of the invention drawn in FIG. 31A;

FIG. 32A is a superior view of one end of an alternative embodiment of the invention drawn in FIG. 31A;

FIG. 32B is a superior view of one end of an alternative embodiment of the invention drawn in FIG. 32A;

FIG. 32C is a superior view of one end of an alternative embodiment of the invention drawn in FIG. 32B;

FIG. 32D is a superior view of one end of the embodiment of the invention drawn in FIG. 32C;

FIG. 32E is a superior view of one end of an alternative embodiment of the invention drawn in FIG. 32D;

FIG. 32F is a superior view of one end of an alternative embodiment of the invention drawn in FIG. 32E;

FIG. 32G is a superior view of one end of an alternative embodiment of the invention drawn in FIG. 32F;

FIG. 32H is a superior view of one end of alternative embodiments of the invention drawn in FIGS. 23E and 32G;

FIG. 33A is a superior view of an alternative embodiment of the needle-like component invention drawn in FIG. 28C;

FIG. 33B is an anterior view of the embodiment of the invention drawn in FIG. 33A;

FIG. 33C is a lateral view of the pointed end of the embodiment of the invention drawn in FIG. 33B and a portion of one end of the embodiment of the invention drawn in FIG. 32G;

FIG. 33D is a lateral view of the pointed end of an alternative embodiment of the invention drawn in FIG. 33C and a portion of one end of the embodiment of the invention drawn in FIG. 32G;

FIG. 34 is a superior view of the proximal end of an alternative embodiment of the invention drawn in FIG. 24E;

FIG. 35A is a superior view of an alternative embodiment of the invention drawn in FIG. 24B and an axial cross section of a portion of the AF;

FIG. 35B is a superior view of the embodiment of the invention and axial cross section of the portion of the AF drawn in FIG. 35A;

FIG. 35C is a lateral view of a partial cross section of the embodiment of the invention drawn in FIG. 35B;

FIG. 35D is a superior view of the embodiments of the invention drawn in FIGS. 5A and 35C and axial cross section of the portion of the AF;

FIG. 35E is a superior view of the embodiment of the invention and axial cross section of the portion of the AF drawn in FIG. 35D;

FIG. 35F is a superior view of the embodiment of the invention and axial cross section of the portion of the AF drawn in FIG. 35E;

FIG. 35G is a superior view of the embodiment of the invention and axial cross section of the portion of the AF drawn in FIG. 35F;

FIG. 35H is a superior view of the embodiment of the invention and axial cross section of the portion of the AF drawn in FIG. 35G;

FIG. 35I is a superior view of an alternative embodiment of the invention drawn in FIG. 35A;

FIG. 35J is a superior view of the embodiment of the invention drawn in FIG. 35I;

FIG. 35K is a lateral view of the distal end of an alternative embodiment of the invention drawn in FIG. 35C;

FIG. 35L is a superior view of the embodiment of the invention drawn in FIG. 35K and an axial cross section of a portion of the AF;

FIG. 35M is a superior view of the distal end of an alternative embodiment of the invention drawn in FIG. 35K;

FIG. 35N is a lateral view of the distal end of an alternative embodiment of the invention drawn in FIG. 35C;

FIG. 35O is a lateral view of the distal end of the embodiment of the invention drawn in FIG. 35N;

FIG. 35P is a lateral view of the distal end of an alternative embodiment of the invention drawn in FIG. 35O;

FIG. 35Q is a lateral view of the distal end of the embodiment of the invention drawn in FIG. 35P;

FIG. 35R is a lateral view of the distal end of an alternative embodiment of the invention drawn in FIG. 35Q;

FIG. 35S is a lateral view of the distal end of an alternative embodiment of the invention drawn in FIG. 35R;

FIG. 36A is a superior view of an alternative embodiment of the invention drawn in 35L and an axial cross section of a portion of the AF;

FIG. 36B is a superior view of the embodiment of the invention an axial cross section of the portion of the AF drawn in FIG. 36A;

FIG. 37A is a superior view of the distal end of an alternative embodiment of the invention drawn in FIG. 36B;

FIG. 37B is a superior view of an alternative embodiment of the invention drawn in FIG. 37A;

FIG. 37C is a superior view of an alternative embodiment of the invention drawn in FIG. 37B;

FIG. 37D is a superior view of an alternative embodiment of the invention drawn in FIG. 37C;

FIG. 38A is a superior view of an alternative embodiment of the invention drawn in FIG. 37D and an axial cross section of a portion of the AF;

FIG. 38B is a superior view of the embodiment of the invention and axial cross section of the portion of the AF drawn in FIG. 38A;

FIG. 38C is a lateral view of the embodiment of the invention drawn in FIG. 38B and a sagittal cross section of a portion of the spine;

FIG. 38D is a superior view of an alternative embodiment of the invention drawn in FIG. 38B and an axial cross section of a portion of the AF;

FIG. 38E is a superior view of an alternative embodiment of the invention drawn in FIG. 38D and an axial cross section of a portion of the AF;

FIG. 38F is a superior view of an alternative embodiment of the invention drawn in FIG. 38E and an axial cross section of a portion of the AF;

FIG. 38G is a superior view of an alternative embodiment of the invention drawn in FIG. 38F and an axial cross section of a portion of the AF;

FIG. 38H is a superior view of an alternative embodiment of the invention drawn in FIG. 38G and an axial cross section of a portion of the AF;

FIG. 38I is a superior view of the embodiment of the invention and axial cross section of the portion of the AF drawn in FIG. 38H;

FIG. 39A is a superior view of the distal end of an alternative embodiment of the invention drawn in FIG. 36A;

FIG. 39B is a lateral view of the distal end of the embodiment of the invention drawn in FIG. 39A;

FIG. 39C is a superior view of the distal end of the embodiment of the invention drawn in FIG. 39B and an axial cross section of an IVD;

FIG. 39D is a superior view of the distal end of the embodiment of the invention and axial cross section of the IVD drawn in FIG. 39C;

FIG. 40A is a superior view of an alternative embodiment of the invention drawn in FIG. 39A;

FIG. 40B is a superior view of the embodiment of the invention drawn in FIG. 40A and an axial cross section of an IVD;

FIG. 40C is a superior view of the embodiment of the invention and axial cross section of the IVD drawn in FIG. 40B;

FIG. 40D is a superior view of the embodiment of the invention and axial cross section of the IVD drawn in FIG. 40C;

FIG. 41A is a lateral view of the distal end of an alternative embodiment of the invention drawn in FIG. 40A;

FIG. 41B is a lateral view of the distal end of the embodiment of the invention drawn in FIG. 41A;

FIG. 41C is a superior view of the distal end of the embodiment of the invention drawn in FIG. 41B and an axial cross section of a portion of the AF;

FIG. 41D is a lateral view of the distal end of an alternative embodiment of the invention drawn in FIG. 41B;

FIG. 41E is an anterior view of the distal end of an alternative embodiment of the invention drawn in FIG. 41A;

FIG. 41F is a superior view of a longitudinal cross section of the distal end of the embodiment of the invention drawn in FIG. 41E;

FIG. 41G is a superior view of a longitudinal cross section of the distal end of the embodiment of the invention drawn in FIG. 41F;

FIG. 42A is a lateral view of a partial transverse cross section of an alternative embodiment of the invention drawn in FIG. 35C;

FIG. 42B is a superior view of a longitudinal cross section of the distal end of the embodiment of the invention drawn in FIG. 42A;

FIG. 42C is a superior view of the distal end of the embodiment of the invention drawn in FIG. 42B;

FIG. 42D is a lateral view of an alternative embodiment of the invention drawn in FIG. 42A;

FIG. 42E is a superior view of a longitudinal cross section of the embodiment of the invention drawn in FIG. 42D and an axial cross section of a portion of the AF;

FIG. 42F is a superior view of a longitudinal cross section of the embodiment of the invention and axial cross section of the portion of the AF drawn in FIG. 42E;

FIG. 42G is a superior view of the distal end of an alternative embodiment of the invention drawn in FIG. 42F;

FIG. 42H is a lateral view of the distal end of the embodiment of the invention drawn in FIG. 42G;

FIG. 42I is a superior view of the distal end of the embodiment of the invention drawn in FIG. 42H;

FIG. 42J is a superior view of the distal end of the embodiment of the invention drawn in FIG. 42G and an axial cross section of a portion of the AF;

FIG. 42K is a superior view of the distal end of the embodiment of the invention drawn in FIG. 42J and an axial cross section of a portion of the AF;

FIG. 42L is a superior view of the distal end of the embodiment of the invention drawn in FIG. 42K and an axial cross section of a portion of the AF;

FIG. 42M is a superior view of the distal end of the embodiment of the invention drawn in FIG. 42L and an axial cross section of a portion of the AF;

FIG. 42N is a superior view of the distal end of the embodiment of the invention drawn in FIG. 42M and an axial cross section of an intervertebral disc;

FIG. 42O is a superior view of the distal end of the embodiment of the invention drawn in FIG. 42N and an axial cross section of a portion of the AF;

FIG. 42P is a lateral view of the distal end of the embodiment of the invention drawn in FIG. 42J and a portion of the AF;

FIG. 42Q is a lateral view of the distal end of the embodiment of the invention drawn in FIG. 42P and a portion of the AF;

FIG. 42R is a lateral view of the distal end of the embodiment of the invention drawn in FIG. 42Q and a portion of the AF;

FIG. 42S is a lateral view of the distal end of the embodiment of the invention drawn in FIG. 42R and a portion of the AF;

FIG. 42T is a lateral view of the distal end of the embodiment of the invention drawn in FIG. 42S and a portion of the AF;

FIG. 42U is a superior view of the distal end of the embodiment of the invention drawn in FIG. 42J;

FIG. 42V is a superior view of the distal end of the embodiment of the invention drawn in FIG. 42U and an axial cross section of a portion of the AF;

FIG. 42W is a superior view of a longitudinal cross section of an alternative embodiment of the invention drawn in FIG. 42F and an axial cross section of a portion of the AF;

FIG. 42X is a superior view of an alternative embodiment of the invention drawn in FIG. 42N;

FIG. 42Y is a view of the distal end of an alternative embodiment of the invention drawn in FIG. 42D;

FIG. 43 is a lateral view of the distal end of an alternative embodiment of the invention drawn in FIG. 41D;

FIG. 44A is superior view of the distal end of an alternative embodiment of the invention drawn in FIG. 24B and an axial cross section of a portion of the AF;

FIG. 44B is a lateral view of the embodiment of the invention drawn in FIG. 44A;

FIG. 44C is a superior view of the distal end of an alternative embodiment of the invention drawn in FIG. 44A and an axial cross section of a portion of the AF;

FIG. 44D is a lateral view of the embodiment of the invention drawn in FIG. 44C;

FIG. 44E is a lateral view of the embodiment of the invention drawn in FIG. 44D, the pointed end of a needle, and a cable loop, which contains an elastic projection, such as a knot;

FIG. 44F is a lateral view of the embodiment of the invention drawn in FIG. 44E;

FIG. 44G is a lateral view of an alternative embodiment of the invention drawn in FIG. 44F;

FIG. 45 is a superior view of the distal end of an alternative embodiment of the invention drawn in FIG. 44C;

FIG. 46A is a superior view of an alternative embodiment of the invention drawn in FIG. 38A and an axial cross section of a portion of the AF;

FIG. 46B is a superior view of the embodiment of the invention and the portion of the AF drawn in FIG. 46A;

FIG. 47A is a superior view of an alternative embodiment of the invention drawn in FIG. 46A and an axial cross section of a portion of the AF;

FIG. 47B is a superior view of the embodiment of the invention and the portion of the AF drawn in FIG. 47A;

FIG. 48A is a superior view of the distal end of an alternative embodiment of the invention drawn in FIG. 22A;

FIG. 48B is a superior view of the embodiment of the invention drawn in FIG. 48A;

FIG. 48C is a superior view of the distal end of an alternative embodiment of the invention drawn in FIG. 48A;

FIG. 48D is a superior view of the distal end of an alternative embodiment of the invention drawn in FIG. 48C;

FIG. 49A is a superior view of alternative embodiments of the inventions drawn in FIGS. 6L and 11F;

FIG. 49B is a superior view of the embodiment of the invention drawn in FIG. 49A and an axial cross section of a portion of the AF;

FIG. 49C is a superior view of the embodiment of the invention and axial cross section of the portion of the AF drawn in FIG. 49B;

FIG. 50A is a superior view of an alternative embodiment of the invention drawn in FIG. 44A and an axial cross section of a portion of the AF;

FIG. 50B is a superior view of the embodiment of the invention and axial cross section of the portion of the AF drawn in FIG. 50A;

FIG. 50C is a superior view of the embodiment of the invention and axial cross section of the portion of the AF drawn in FIG. 50B;

FIG. 50D is a superior view of the embodiment of the invention and axial cross section of the portion of the AF drawn in FIG. 50C;

FIG. 50E is a superior view of the embodiment of the invention and axial cross section of the portion of the AF drawn in FIG. 50D;

FIG. 50F is a superior view of the embodiment of the invention and axial cross section of the portion of the AF drawn in FIG. 50E;

FIG. 50G is a superior view of a longitudinal cross section of the embodiment of the invention drawn in FIG. 50F;

FIG. 50H is a superior view of a longitudinal cross section of the embodiment of the invention drawn in FIG. 50G;

FIG. 50I is a lateral view of a longitudinal cross section of the embodiment of the invention drawn in FIG. 50H;

FIG. 51 is a superior view of an alternative embodiment of the invention drawn in FIG. 50G and an axial cross section of the portion of the AF;

FIG. 52A is a superior view of a longitudinal cross section of an alternative embodiment of the invention drawn in FIG. 51; and

FIG. 52B is a superior view of a longitudinal cross section of the embodiment of the invention drawn in FIG. 52A.

### DETAILED DESCRIPTION OF THE INVENTION

FIG. 1A is a lateral view of an alternative embodiment of the invention drawn in FIGS. 4A-O of my co-pending patent application U.S. Ser. No. 61/305,683. FIG. 1B is a lateral view of a longitudinal cross section of the embodiment of the invention drawn in FIG. 1A. The shaft 102 of the instrument 100 is preferably 10 to 30 centimeters long, 2 to 8 millimeters in diameter, and made of plastic, such as polypropylene or polyethylene or metal, such as stainless steel, titanium or other such material. A smaller elongate component 104 is fastened to the distal end of the shaft of the tool. The smaller elongate component is preferably 10 to 40 millimeters long, 1 to 3 millimeters in diameter, and made of non-magnetic such as plastic or slightly paramagnetic materials such as titanium. The distal end of the smaller elongate component 104 is fastened near one end of a cylindrical magnet 110. The cylindrical magnet 110 is preferably 2 to 6 millimeters long, 2 to 5 millimeters in diameter, and made of metal coated sintered Grade N52 NdFeB, also known as Neodymium magnets (see MagnetsandMagnets.com, Lakeland, Fla.).

The cylindrical magnet 110 is preferably axially magnetized with the poles on the flat ends of the magnet. The cylinder may be preferably diametrically magnetized in alternative embodiments of the invention. Alternative magnetic material could be used in alternative embodiments of the invention. For example, the component at the distal end of the tool could be made of ferromagnetic materials including iron, nickel, cobalt, or alloys that include such materials. Non-permanent, electro-magnets may be used in alternative embodiments of the invention.

The components of the tool 100 could be smaller or larger than described above and they could be non-cylindrical shapes in alternative embodiments of the invention. The components could be fastened to each other with glue, pins, laser welds, or other fastening mechanisms. The distance between the proximal end of the magnetic cylinder and the distal end of the shaft component is preferably 4 to 15 millimeters long. The axis of the smaller elongate component is preferably co-axial but not co-linear with the axis of the shaft of the tool. Alternatively the tool could be assembled with multiple magnets. For example, the distal horizontal cylinder magnet could be attached to one or more vertically oriented cylinder magnets. The distal most vertical magnet is preferably 2 to 5 millimeters in diameter and 10 to 15 millimeters long. A second larger vertical magnet could be attached to the proximal end of the distal magnet. For example, the larger vertical magnet could be 3 to 15 millimeters in diameter and 10 to 100 millimeters long or longer. A series of large diameter magnets could be attached to the proximal end of the small diameter magnet. FIG. 1C is an anterior view of the embodiment of the invention drawn in FIG. 1B.

FIG. 2 is an oblique view of an alternative embodiment of the invention drawn in FIG. 1A. A cable 202 is seen passing through a hole 204 through a generally cylindrical component 206. The ends of the cable are held with an elongate crimped component 208. The cylinder component 206 is preferably 1 to 2 millimeters in diameter, 2 to 5 millimeters long, and preferably is a magnet made of magnetic material listed in the text of FIG. 1B or, less preferably is not a magnet but made of ferromagnetic material such as iron, nickel, cobalt, steel or other such material. The magnet is preferably axially magnetized but may be diametrically magnetized in alternative embodiments of the invention. The cable 202 is preferably 0.1 to 0.4 millimeters in diameter, 2 to 20 centimeters long, and made of stainless steel or other high tensile strength material. The cable may be coated in nylon.

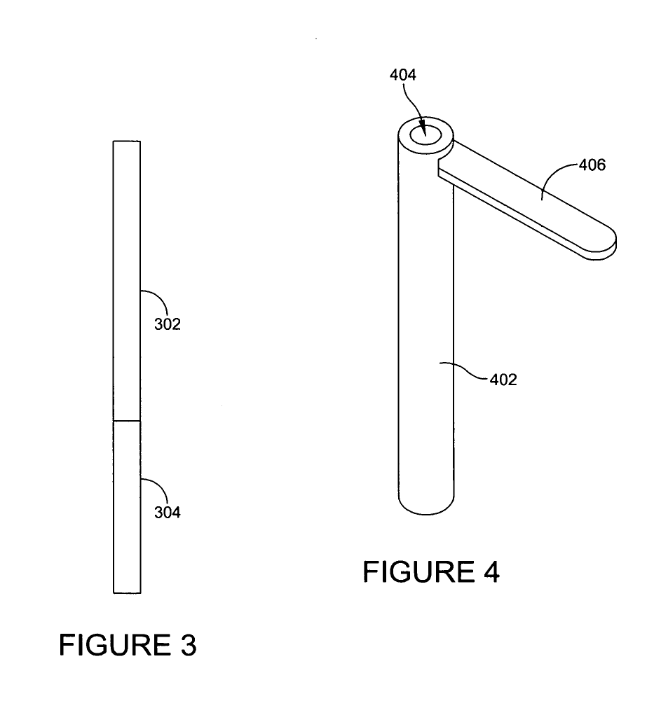

FIG. 3 is a lateral view of an alternative embodiment of the invention drawn in FIG. 1A. A generally cylindrical magnet 304 is seen fastened to the distal end of a generally cylindrical shaft component 302. The cylindrical magnet is preferably 2 to 15 millimeters in diameter and 10 to 200 millimeters long or longer. The direction of magnetization is preferably axial and the magnet is preferably made of one of the previously listed materials. The magnet is most preferably 50 or more millimeters long. Increasing the length or the diameter of the magnet increases the field strength of the magnet.

The elongate shaft component 302 is preferably made of a non-magnetic material, such as plastic. The two components may be glued, pined, or otherwise fastened together. Multiple axially magnetized generally cylindrical magnets could be connected in series to increase the magnetic attraction force at the end of the assembled magnetic tool. For example, surgeons could add 1, 2, 3, 4, 5, 6, 7, or more 50 millimeters long magnets in series to increase the magnetic attraction force. The stacked magnets could have the same or different diameters. If different diameter magnets are stacked, the distal most magnet is preferably 2 to 5 millimeters in diameter and at least 10 millimeters long.

FIG. 4 is an oblique lateral view of an embodiment of the invention used with the embodiment of the invention drawn in FIG. 3. The inner diameter 404 of the cannulated tool 402 is preferably slightly larger than the outer diameter of the tool drawn in FIG. 3. The shaft of the cannulated tool is preferably 110 to 20 centimeters long and shorter than the length of the tool drawn in FIG. 3. The cannulated tool is preferably made of a non-magnetic material, such as plastic. The tool drawn in FIG. 3 is passed through the lumen 404 of the cannulated tool 402. Axial magnetization of the magnet in the tool drawn in FIG. 3 and shielding of the magnet with the tool drawn in FIG. 4 reduce the strength of the magnetic field that surrounds the sides of the elongate magnet. The outer diameter of the cannulated tool is preferably 2 to 10 millimeters larger than the inner diameter of the tool. A handle 406 extends from the side of the cannulated tool.

FIG. 5A is an anterior view of an embodiment of the invention used with the embodiment of the invention drawn in FIG. 2. FIG. 5B is an anterior view of a longitudinal cross section of the embodiment of the invention drawn in FIG. 5A and an anterior view of the embodiment of the invention drawn in FIG. 2. The cylindrical magnet of the embodiment of the invention drawn in FIG. 2 is seen within the lumen of a needle-like component 502 at the distal end of the embodiment of the invention drawn in FIG. 5A. The cable of the embodiment of the invention drawn in FIG. 2 is seen extending through a slot in the needle-like component 502. A stylet 510 is seen proximal to the cylinder magnet component. The stylet is pushed towards the distal end of the needle component to expel the cylinder magnet from the needle-like component.

The proximal end of the needle-like component is fastened to the distal end of the shaft component of the tool. The proximal end of the shaft component is seen within a handle component 512. The inner diameter of the needle-component is preferably slightly larger than the outer diameter of the cylinder magnet. For example, the inner diameter of the needle component could be about 1.1 millimeter in diameter. The outer diameter of the needle component is preferably less than 2.0 millimeters in diameter. The needle component is preferably 5 to 20 millimeters in length and made of hard material such as steel, stainless steel, or titanium. The shaft of the stylet is preferably slightly smaller than the inner diameter of the needle-like component and 10 to 30 centimeters long and preferably made of titanium or plastic, but may be made of stainless steel.

The stylet, or a component attached to the distal end of the stylet could be magnetized. In the magnetized embodiment, the stylet preferably forces the cylinder magnet component from the needle. For example, the distal end of the stylet could have same polarity as the proximal end of the cylinder magnet. The stylet component is preferably about 3 to 10 millimeters longer than shaft and needle component of the tool drawn in FIG. 5B. The inner diameter of the shaft of the tool is slightly larger than the diameter of the cylinder magnet. The outer diameter of the shaft component is preferably 1 to 10 millimeters larger than the inner diameter of the shaft component.

FIG. 6A is a lateral view of the distal ends of the embodiments of the invention drawn in FIGS. 1A and 5B and a superior view of an axial cross section of a portion of the AF. The cylinder magnet of the tool drawn in FIG. 1A was inserted through a fissure in the AF then rotated 90 degrees. The distal end of the tool drawn in FIG. 5B was pushed through the AF preferably 2 to 8 millimeters lateral to the fissure. The distal end of the tool drawn in FIG. 5B was pushed against the AF which, assures the distal end of the needle component is inserted 5 to 15 millimeters into the IVD.

Additional axially magnetized generally cylindrical magnets may be added to the end of the magnet, after the distal end of the tool is inserted through the fissure. Such addition of magnets extends the reach and the attractive force of the magnet. The axially magnetized cylindrical magnets align with one another in the IVD. Placement of a tool in the fissure helps surgeons optimize placement of the cable component one to several millimeters lateral to the fissure. A cannulated guide tool could be inserted over the shaft of the embodiment of the invention drawn in FIG. 1A. The shaft of the embodiment of the invention drawn in FIG. 5A could be inserted through a second lumen in the guide tool. The novel guide could be used to insure the cylindrical component of the cable component is placed near or on one end of the magnetic component of the embodiment of the invention drawn in FIG. 1A.

FIG. 6B is a lateral view of the distal ends of the embodiments of the invention drawn in FIGS. 1A & 5B and a superior view of an axial cross section of a portion of the AF. The stylet component of the tool drawn in FIG. 5B was pushed in a distal direction, which force the magnet cylinder from the needle-like component and into the IVD, then the insertion tool was removed from the IVD. Attraction between the ends of the axially magnetized cylinder components aligns the axes of the magnet component coaxial and generally collinear with each other. Dissection between the AF and the NP with a right angle probe could facilitate alignment of the two cylinder magnetic components. Pulling the FIG. 1A tool from the fissure preferably pulls the cylinder magnet component of the tool drawn in FIG. 2 through the fissure.

FIG. 6C is a lateral view of the distal ends of the embodiments of the invention drawn in FIGS. 2 to 4 and a superior view of an axial cross section of a portion of the AF. The distal end of the cylindrical magnet of the tool drawn in FIG. 3 is seen extending beyond the distal end of the shield tool drawn in FIG. 4 and through a fissure in the AF. The end of the magnet component drawn in FIG. 2 is seen attached to the end of the cylindrical magnet inserted through the fissure. The embodiment of the invention is used to pull the cylinder magnet of FIG. 2 through the fissure, if the tool drawn in FIG. 1A fails to do so. The axes of axially magnetized cylinders align to minimize the width of the coupled magnets, which facilitates extraction of the magnets through the fissure. The strong attraction force of multiple stacked magnets preferably enables attraction of the cylinder magnet on the cable loop through the AF. In such embodiment the stacked magnet tool could be used to move the cable loop to then through the fissure without inserting the distal end of the stacked magnet through the fissure.

Larger diameter magnets could be stacked on the proximal end of smaller diameter magnet in certain preferred embodiments of the invention. For example, one or more ten millimeter diameter cylindrical magnets could be stacked on the proximal end of a three millimeter diameter, ten millimeter long cylindrical magnet. The stacked magnets may preferably be made of different materials. Such combinations may increase the magnetic force of cylinder magnet with a small enough diameter for insertion through a fissure, by magnets too large to insert into the fissure. The proximal magnets are preferably stacked on the distal magnet after placement of the distal on or in the AF. This embodiment of the invention enables surgeons to slowly and incrementally increase the attraction force exerted by the distal magnet.

The insertion tool could include a component, such as a LED or light bulb, that illuminates when an electrical circuit is completed when the magnet on the cable contacts the magnet on the tool. The end flexible longitudinal fixation component is seen passing through the proximal end of the cable loop. For example, a #2 weldable braided polyester suture flexible longitudinal fixation component (Tornier, Edina Minn.) could be used in preferred embodiments of the invention. Additional flexible longitudinal fixation component materials are described in my co-pending patent applications including U.S. Ser. Nos. 61/300,993 and 61/305,683 as well as my co-pending patent application PCT/US2009/065954 could be used in this embodiment of the invention.

FIG. 6D is lateral view of the flexible longitudinal fixation component 602 drawn in FIG. 6C and a superior view of an axial cross section of a portion of the AF. The magnetic cylinder of the component drawn in FIG. 2 was pulled through the fissure then the central portions of the arms of the flexible longitudinal fixation component was cut, which releases the cable loop. FIG. 6E is a lateral view of the distal end of alternative embodiments of the invention drawn in FIGS. 6C and 6D and a superior view of an axial cross section of a portion of the AF. A second magnet and cable loop, such as the component drawn in FIG. 2 was passed through AF tissue then through the fissure using the invention drawn in FIG. 6A-6D. FIG. 6F is a lateral view of the embodiment of the invention drawn in FIG. 6E and a superior view of an axial cross section of a portion of the AF. The cable loop with the cylinder magnet was cut and removed, then the end of one of the flexible longitudinal fixation components that pass through the fissure was also passed through a cable loop. FIG. 6G is a lateral view of the embodiment of the invention drawn in FIG. 6F and a superior view of an axial cross section of a portion of the AF. The cable loop was pulled from the IVD, which pulls an end of one of the flexible longitudinal fixation components through a surgically created aperture in the AF lateral to the fissure.

FIG. 6H is a lateral view of the embodiment of the invention drawn in FIG. 6G and a superior view of an axial cross section of a portion of the A. The end of the second flexible longitudinal fixation component that was passed through the fissure was passed through AF tissue lateral to the fissure using the embodiment of the invention drawn in FIGS. 6E-6G. FIG. 6I is a posterior view of the embodiment of the invention and IVD drawn in FIG. 6H. First ends of the flexible longitudinal fixation components pass through a single surgically created aperture in the AF 2 to 8 millimeters lateral to the first side of the fissure and seconds ends of such fixation components pass through separate surgically created apertures in the AF 2 to 8 millimeters lateral to the second side of the fissure.

FIG. 6J is a posterior view of the embodiment of the invention and IVD drawn in FIG. 6I. The ends of the flexible longitudinal fixation components 602, 602′ were welded together (Tornier, Edina, Minn.). Tension on the first and second ends of each fixation component before welding, closes or narrows the fissure 610. The invention closes the fissure with two separate welded flexible longitudinal fixation components. The flexible longitudinal fixation components could be configured differently in alternative embodiments of the invention. For example, the ends of the flexible longitudinal fixation components could pass through single surgically created apertures in the AF on both sides of the fissure.

FIG. 6K is a superior view of an axial cross section of the embodiment of the invention and a portion of the AF drawn in FIG. 6J. The edges of the fissure are preferably apposed by tension on the flexible longitudinal fixation component(s).

FIG. 6L is a superior view of an axial cross section of an alternative embodiment of the invention drawn in FIG. 6K and a portion of the AF. An intra-aperture component 620, such as the intra-aperture components described in my co-pending patent applications U.S. Ser. Nos. 61/300,993 and 61/305,683, the entire content of each being incorporated herein by reference, is seen within the fissure. The second ends of the flexible longitudinal fixation components were passed through the intra-aperture component 620 then passed through separate surgically created apertures in AF tissue lateral to the second side of the fissure.

FIG. 7 is an oblique lateral view of an alternative embodiment of the invention drawn in FIG. 2. The ends of the cable 704 are crimped in a cylindrical magnet component 702. The ends of the cable could be fastened to the cylindrical magnet using other mechanisms or techniques. For example, the ends of the cable could be glued to the end of the cylinder magnet with cyanoacrylate or welded to the end of the end of the magnet 702. FIG. 8A is an oblique lateral view of an alternative embodiment of the invention drawn in FIG. 7. The cable 704 passes through a hole near one end 710 of the magnet 703. The second end 712 of the magnet is an inclined plane.

FIG. 8B is a lateral view of the embodiments of the invention drawn in FIGS. 6B and 8A and a superior view of a portion of the AF. The cylinder magnet was passed through the AF using the embodiment of the invention drawn in FIGS. 5A and 5B. The transverse hole in the cylinder magnet is near one end of the magnet and preferably closer to the shortest side of the magnet. Tension on the end of the cable loop pulls the inclined end of the magnet against the inner side of the AF and causes rotation of the cylinder magnet in a clockwise direction towards the transverse cylinder magnet. The inclined end of the magnet is preferably opposite in polarity to the end of the transverse magnet closest to the magnet with the inclined end. The invention brings the ends of the cylinder magnets close together, which reduces the strength of the magnets required to attract and connect the two components.

FIG. 9A is a lateral view of an alternative embodiment of the invention drawn in FIG. 8A. One end 904 of the magnet 902 is pointed, which enables the magnet to be pushed through the AF without the embodiment of the invention drawn in FIGS. 5A and 5B. The cylindrical magnet is preferably 1 to 2 millimeters in diameter and 2 to 8 millimeters in length. The cylindrical magnet may preferably be curved in alternative embodiments of the invention.

FIG. 9B is a lateral view of a longitudinal cross section of the embodiment of the invention drawn in FIG. 9A and an insertion tool 910. The distal end of the insertion tool is seen in recess in the proximal end of the magnet 902. The wire loop 906 is seen cleated or otherwise fastened to the shaft or the handle 912 of the insertion tool 910. Tension on the cable loop holds the magnet on the tool. The distal end of the insertion tool is preferably made of titanium, plastic, or other minimally magnetic material. Alternatively, that component could be made of magnetic materials. Such distal component if the insertion tool is preferably 5 to 15 millimeters in length.

The proximal end of the distal component of the insertion tool is fastened to the distal end of a shaft component, which preferably has a larger diameter than the diameter of the cylinder magnet. For example, the diameter of the shaft of the tool could be 1 to 5 millimeters larger than the diameter of the cylinder magnet. The distal end of the shaft of the insertion tool preferably strikes the outer surface of the AF when the cylinder magnet is inserted 5 to 15 millimeters into the IVD. The shaft of the insertion tool is preferably 10 to 20 centimeters in length and made of metal, such as stainless steel or titanium or plastic, such as polyethylene or polypropylene. The tool is used to push the magnet through AF tissue. Then the flexible longitudinal fixation components are passed through the AF as described in the embodiments of the invention drawn in FIGS. 6B-6K.

FIG. 10A is a lateral view of an alternative embodiment of the invention drawn in FIG. 9A. The cable 1006 passes through hole 1004 in a projection 1005 from the proximal end of the magnet 1002. FIG. 10B is a lateral view of the embodiment of the invention drawn in FIG. 10A and an alternative embodiment of the insertion tool drawn in FIG. 9B. The projection from the proximal end of the magnet fits in the lumen 1010 of the shaft of a cannulated insertion tool 1012. The cable passes through the lumen of the shaft of the insertion tool

FIG. 11A is a posterior view of an alternative embodiment of the invention drawn in FIG. 6E and a coronal cross section of a portion of the spine. An anchor 1004 with a flexible longitudinal fixation component 1106 was placed in the cranial end of a vertebra 1102. A magnet 1110 and cable loop 1112 were passed through AF tissue cranial to a transverse fissure 1120 near the vertebra using the embodiments of the invention drawn in FIGS. 6A-6C.

FIG. 11B is a posterior view of the embodiment of the invention and coronal cross section of a portion of the spine drawn in FIG. 11A. The cranial end of the flexible longitudinal fixation component was passed through the cranial end of the cable loop, then the cable loop was pulled from the IVD, which pulls the cranial end of the fixation component through the fissure. FIG. 11C is a posterior view of the embodiment of the invention and coronal cross section of the portion of the spine drawn in FIG. 11B. A second magnet 1112′ and cable 1114′ loop were passed through the AF tissue cranial to the fissure using the embodiment of the invention drawn in FIG. 6E.

FIG. 11D is a posterior exploded view of the embodiment of the invention and coronal cross section of a portion of the spine drawn in FIG. 11C. The cable loop was pulled through the fissure then the cable loop was cut and removed. The cranial end of the flexible longitudinal fixation component was passed through a cable loop 1122. A sleeve component 1124 will be placed over the end of the fixation component in the next step of the procedure. The sleeve component preferably has an inner diameter of 0.3 to 2.0 millimeters, an outer diameter of 0.5 to 3.0 millimeters, and is 1 to 10 millimeters long. The sleeve component is preferably made of metal, plastic, or bioabsorbable materials. The sleeve is advanced beyond the cable loop.

FIG. 11E is a posterior view of the embodiment of the invention and coronal cross section of a portion of the spine drawn in FIG. 11D. The cable loop was pulled from the IVD, which pulls the cranial end of the fixation component through AF tissue cranial to the fissure a second time. The ends of the flexible longitudinal fixation component are then welded together at 1130. FIG. 11F is a partial superior view of and axial cross section of the embodiment of the invention and a portion of the AF drawn FIG. 11E. The sleeve 1124 lies behind the inner layer 1125 of the AF and prevents tension on the flexible longitudinal fixation component from pulling such portion of the AF tissue together.

FIG. 11G is a lateral view of a partial sagittal cross section of the embodiment of the invention and portion of the spine drawn in FIG. 11E. Threaded or push-in anchors 1150 described in my co-pending patent applications including U.S. Ser. Nos. 61/300,993 and 61/305,683 as well as my co-pending patent application PCT/US2009/065954 could be used in this embodiment of the invention. The entire content of all of the references are incorporated herein by reference.

FIG. 12A is a lateral view of an alternative embodiment of the invention drawn in FIG. 7. The ends of the cable 1204 are fastened to a metal, preferably magnetic generally cylindrical component 1202. For example, a cylindrical component could be crimped to hold the ends of the cable. Alternatively, the end of the cable could be glued to or in the cylindrical component with an adhesive, such as Loctite 39205 with Loctite 7380, Loctite 3032 with Loctite primer 770, JB Weld epoxy, Liquid Nails, Gorilla Glue, or cyanoacrylate. Alternative methods, such as laser welding could be used to fasten the components. The end of the cylindrical component farthest from the ends of the cable is preferably conical shaped. The opposite end or both ends of the cylindrical component could be conical shaped in alternative embodiments of the invention. As previously described, the component 1202 is preferably a cylindrical magnet, but if not a magnet, it is preferably made of ferromagnetic material.

FIG. 12B is a superior view of the embodiment of the invention drawn in FIG. 12A and an axial cross section of a portion of the AF. The cylindrical end of the embodiment of the invention drawn in FIG. 12A was placed through a surgically created aperture lateral to a fissure 1210 in the AF. For example, the cable component could be passed through the AF using the embodiments of the invention drawn in FIGS. 5A, 5B, and 6A-6D. FIG. 12C is a superior view of the embodiment of the invention and axial cross section of the portion of the AF drawn in FIG. 12B. A second cable component was passed through a surgically created aperture in the AF on a second side of the fissure using the embodiment of the invention described in FIG. 12B.

FIG. 12D is a superior view of the embodiment of the invention and axial cross section of the portion of the AF drawn in FIG. 12C. Tension on one arm of the second cable loop increases the opening in the cable loop near the cylindrical component. A tool 1220 is seen applying tension to one arm of the second cable loop. Alternatively, tension could be applied to an arm of the second cable loop near the surgically created aperture. FIG. 12E is a superior view of the embodiment of the invention and axial cross section of the portion of the AF drawn in FIG. 12D. The cylindrical component of the first cable component was passed through the opening between the cable arms of the second cable component. The elasticity of the cable helps hold the cable components in such configuration.

FIG. 12F is a superior view of the embodiment of the invention and axial cross section of the portion of the AF drawn in FIG. 12E. Tension on the lateral end of the second cable loop pulls the cylinder ends of both cable loops into the fissure. FIG. 12G is an exploded superior view of the embodiment of the invention and axial cross section of the portion of the AF drawn in FIG. 12F. Tension on the lateral end of the second cable loop pulled the second cable loop from the IVD and pulled the cylindrical component of the first cable loop or component through the second surgically created apertures.

FIG. 12H is a superior view of the embodiment of the invention and axial cross section of a portion of the AF drawn in FIG. 12G. Tension on the cylindrical component of the first cable component from the IVD, which pulls the central portion of the flexible longitudinal fixation component 1230 through the first then the second surgically created apertures. The central portion of the flexible longitudinal fixation component 1230 is cut to release the cable component and to form two flexible longitudinal fixation components. Tension is preferably applied to the ends of each flexible longitudinal fixation component, to close or narrow the fissure, then the ends of each flexible fixation components are fastened to each other, for example by welding such components.

FIG. 13A is an oblique lateral view of an alternative embodiment of the invention drawn in FIG. 12A. The ends of the cable component 1304 are fastened to the outside of a magnet or ferromagnetic component 1302. For example, the ends of the cable could be fastened to a generally cylindrical component using the adhesives and other techniques previously described. The magnet or ferromagnetic component could be shapes other than cylindrical in all embodiments of the invention. For example, such components could be elongate but triangular, square, rectangular, hexagonal, or other shape in cross section.

FIG. 13B is a superior view of the embodiment of the invention drawn in FIG. 13A and a portion of the AF. The cable components were passed through surgically created apertures in the AF then the fissure using the embodiments of the invention described in FIGS. 5A, 5B, and 6A-6D. First ends of flexible longitudinal fixation components 1310, 1312 were passed through the cable components.

FIG. 13C is an exploded superior view of the embodiment of the invention and portion of the AF drawn in FIG. 13B. Tension on the cylindrical components or the cables near the cylindrical components pulled the cable components through the surgically created apertures, then through the fissure, which pulled the first ends of the flexible fixation members through such openings it the AF. The flexible fixation components were released from the cable components as the cable components were pulled away from the IVD.

FIG. 13D is a superior view of the embodiment of the invention and portion of the AF drawn in FIG. 13C. The first ends of the flexible fixation components were fastened together, for example by tying a knot 1314. Alternative methods could be used to fasten the components together, such as clips, welds, adhesives, or other methods.

FIG. 13E is a superior view of the embodiment of the invention and portion of the AF drawn in FIG. 13D. Tension on the second ends of the flexible longitudinal fixation components pulls the knot 1314 into and possibly through the fissure. The second ends of the flexible longitudinal fixation components were welded under tension then cut to remove excess flexible fixation material. The second ends of the flexible fixation components could be fastened together using other methods, such as the methods previously described.

FIG. 14 is a lateral view of an alternative embodiment of the invention drawn in FIG. 13A. The ends of the cable or flexible member 1402 are fastened to different sides of the cylindrical component 1404 using the previously described methods. FIG. 15A is a lateral view of an alternative embodiment of the invention drawn in FIG. 14. The ends of the cable or flexible elongate member 1502 are fastened to the end of the cylindrical component 1502 using the previously described methods. FIG. 15B is an anterior view of the embodiment of the invention drawn in FIG. 15A.

FIG. 16A is a lateral view of an alternative embodiment of the invention drawn in FIG. 15A. The ends of the cable or flexible elongate member 1604 are fastened to a projection from the cylindrical component 1602. For example, the projection could be crimped to hold the ends of the cable in a slot or hole in the projection. The previously described methods could be used to fasten the cable to the projection in alternative embodiments of the invention. Alternatively, the ends of the cable could be cleated into a slot in the projection.

FIG. 16B is a lateral view of an alternative embodiment of the invention drawn in FIG. 16A. One end of the cable was passed through a hole or slot in the projection of the cylindrical component then passed into an elongate, preferably cylindrical component 1606 to fasten the ends of the cable together.

FIG. 16C is a lateral view of an alternative embodiment of the invention drawn in FIG. 16B. The ends of the cable were fastened to or in a hole, holes, slot or slots in a projection 1603 using the previously described techniques. FIG. 17 is a lateral view of an alternative embodiment of the invention drawn in FIG. 16C. The ends of the cable were fastened to or in a hole, holes, slot, or slots in one end of the cylindrical component 1702.

FIG. 18 is a lateral view of an alternative embodiment of the invention drawn in FIG. 10A. The ends of the cable 1804 were fastened to a first component 1803, which was fastened to an elongate, generally cylindrical magnet or ferromagnetic component. For example, the ends of the cable could be crimped into a metal cylindrical component 1803 that is then glued or welded to the magnetic component 1802. Alternatively, the components could be fastened together using the previously described fastening methods. This embodiment of the invention could be incorporated into other embodiments of the invention, such as the embodiments of the invention drawn in FIGS. 2, 7, 8A, 9A, 10A, 12A, 13A, 14, 15A, 16A-16C, and 17.

FIG. 19 is a lateral view of an alternative embodiment of the invention drawn in FIG. 14. A flexible elongate component, preferably 0.05 to 1.0 millimeters in diameter, is wrapped around the ends of the cable and the cylindrical magnet or ferromagnetic component 1902 at 1906 to fasten the components together. The coiled elongate component 1904 and the cylindrical component 1902 could be coated with adhesive, such as the epoxy adhesives previously described, to increase the strength of the connection between the components.

FIG. 20 is a lateral view of an alternative embodiment of the invention drawn in FIG. 19. The ends of the elongate cable-like component 2004 were threaded through two cylinder ring-like component 2003, then fastened to a third ring-like component 2002. One or more of the ring-like components are preferably magnets or made of ferromagnetic material. The ends of the cable could be fastened to two or more of the ring-like components in alternative embodiments of the invention.

FIG. 21A is a superior view of a partial cross section of the distal end of an alternative embodiment of the invention drawn in FIG. 6A and a portion of the AF. A magnet 2102 is seen at the end of an elongate component 2103. The elongate component 2103 is seen in a tube-like component 2104 that was passed through a fissure in the AF. The elongate component is preferably of a shape memory material such as Nitinol. Alternatively, the elongate component could be made material such as stainless steel or titanium assuming the same are “elastic” in the sense that they spring back from a deformed condition. The elongate component is seen its constrained or first shape. The elastic elongate component is preferably 0.5 to 3.5 millimeters in diameter and about 20 millimeters long. The outer diameter of the tube-like component 2104 is preferably between 2 and 6 millimeters in diameter and about 0.4 to 2.0 millimeters larger than the inner diameter of the tube. The tube is preferably about 0.5 to 2 centimeters shorter than the elastic elongate component. The tube is preferably made of metal, such as stainless steel, or plastic.

FIG. 21B is a superior view of the partial cross section of the embodiment of the invention and portion of the AF drawn in FIG. 21A. The elongate component was pushed distally into the IVD, which released the constrained elongate component. The released elongate component preferably bends between 45 to 110 degrees, most preferably about 90 degrees, which pushes the magnet component through the nucleus pulposus tissue and under the inner AF lateral to the fissure. The distal end of the magnet preferably extends 2 to 10 millimeters lateral to the fissure. The horizontal portion of the distal end of the deployed elastic elongate component is preferably 1 to 8 millimeters long.

FIG. 21C is a superior view of a partial cross section of the distal end of the embodiment of the invention and portion of the AF drawn in FIG. 21B and a superior view of the embodiment of the invention drawn in FIG. 12A. The cylinder component 1202 was passed through a surgically created aperture in the AF using the embodiments of the invention drawn in FIGS. 5A, 5B, and 6A-6D. After the magnets 1202, 2102 are attracted to one another, the elastic elongate component 2103 was pulled in a proximal direction into the tube, which constrains the elastic component. The tube, elastic component, magnets, and cable are pulled from the IVD after one end of a flexible longitudinal fixation component is passed through the opening formed by the cable. An additional flexible longitudinal fixation component is passed through a surgically created aperture on a second side of the fissure in the next step of the procedure, then the ends of the flexible fixation components are welded together using the embodiment of the invention drawn in FIGS. 12B to 12H.

FIG. 22A is a superior view of the distal end of alternative embodiments of the inventions drawn in FIG. 5A and FIG. 1N of my co-pending patent application 61/381,585 entitled “SOFT TISSUE REPAIR METHODS AND APPARATUS,” the entire content of which is incorporated herein by reference. A slot 2204 near the pointed end 2203 of the needle component 2202 faces the shaft of the tool. The slot is preferably 0.1 to 1.0 millimeters wide and 0.1 to 3 millimeters deep. The inlet of the slot is preferably within 1 to 4 millimeters from the end of the point of the needle. The needle is preferably made of stainless steel, titanium, or a shape memory material such as Nitinol. The needle is preferably 0.5 to 2.0 millimeters in diameter. The other dimensions of the needle component and the dimensions and materials of the other components are described in my co-pending patent applications such as 61/381,585.

An outer most tube component 2210 is seen over the outer tube component 2208 drawn in FIG. 1N of my co-pending application 61/381,585. The inner diameter is slightly larger than the outer diameter of the tube inside the outer most tube. The outer diameter of the outer most tube is preferably about 0.4 to 1.0 millimeters larger than the inner diameter of the tube. The outer most tube is preferably 10 to 40 millimeters shorter than the tubes inside the outer most tube.

The proximal end of the outer most tube 2210 is fastened to the proximal end of the tube just inside the outer most tube or to a handle component. For example, such components may be releasably fastened with threads. The outer most component is preferably made of metal, such as stainless steel, or plastic. The outer most tube is forced distally as the needle is deployed or after the needle is deployed. The distal end of the outer most tube applies pressure on the proximal side of the needle to force the needle or hold the needle in the fully deployed position. The outer most tube 2210 is retracted, or moved in a proximal direction, by about 10 to 20 millimeters to enable the needle 2202 to move into the inner tubes when the needle is constrained.

The slot 2220 seen in the side of the distal end of the outer tube may be used to force the deployed needle into the inner tubes. For example, the needle becomes misaligned with the openings in the sides of the inner tubes 2208, the outer most tube can be advanced distally and rotated until the needle passes into the slot in the tube. Further rotation of the tube, in a clockwise direction, forces the needle to align with the windows of the inner tubes, which facilitates constraint of the deployed needle. The proximal end of such slot may extend to beyond the tip of the needle in alternative embodiments of the invention.

FIG. 22B is a superior view of a longitudinal cross section of the distal end of the embodiment of the invention drawn in FIG. 22A. The inner most rod-like component 2206 is pinned to the second most outer tube. The pins pass through a slot in the inner most tube. The needle is deployed with a fully retracted outer most tube by pulling the inner tube in a proximal direction relative to the outer tube and the rod-like component. Such movement aligns the openings in the sides of the inner most two tubes and forces the proximal side of the needle against the distal end of the rod-like component. The outer most tube is seen in its distal position, which helps move and hold the needle in the deployed position. The outer most tube is moved to its proximal most position before the needle is constrained. As described in certain of my co-pending patent applications, the distal edge of the opening in the side of second most inner tube presses on the distal side of the needle to push the needle into the tubes, or constrain the needle, by moving the inner most tube and the needle in a distal direction relative to the second most inner tube.

FIG. 23A is a lateral view of the first end of an alternative embodiment of the invention drawn in FIG. 20. The end of a flexible longitudinal fixation component 2310 was passed through one end of the cable loop 2304. A ring-like component 2303 is seen near the second end of the cable loop. A projection 2302 is seen from the ring-like component. The distal end of the projection preferably extends toward the flexible longitudinal fixation component. The projection is preferably 0.5 to 2 millimeters long and 0.2 to 1.0 millimeters in diameter.

The ring-like component 2303 is preferably made of elastic material, such as metal, including stainless steel, titanium, or Nitinol, or plastic including polyethylene, nylon, or polypropylene. The ring-like component 2303 may slide along the cable-like component, if at least a few pounds of force are applied to pull the cable component through the ring-like component or be the two components may be fastened together with adhesive or by crimping the ring-like component. As previously described, the cable loop component is preferably 10 millimeters to 20 centimeters long and made of high tensile strength multi-filament material that is 0.1 to 1.0 in diameter. The flexible longitudinal fixation component is preferably 60 to 96 inches long and about 0.3 to 0.8 millimeters in diameter.

FIG. 23B is a superior view of the embodiments of the invention drawn in FIGS. 22A and 23A and a partial axial cross section of a portion of the AF. The first end of the cable component was loaded into the slot in the side of the deployed needle, then the needle was constrained, which pulls the captured portion of the cable into the tubes if the insertion tool. The distal end of the insertion tool was then passed through a fissure in the AF, then the needle was deployed and the instrument was pulled away from the IVD, which forces the point of the needle through the AF.

FIG. 23C is a superior view of the embodiment of the invention and portion of the AF drawn in FIG. 23B. The insertion tool was pushed towards the center of the disc, then the needle was constrained and the insertion tool pulled from the IVD. The cable slid out of the slot in the needle as the needle was pulled into the IVD. The projection 2302 on the ring-like component 2303 of the cable impinges on the outer surface of the AF as the cable component is pulled towards the center of the IVD, which helps the cable slide our to the slot in the needle. The elastic projection component preferably moves toward the cable as the ring-like component is pulled through the AF, then moves away from the cable as the ring-like component passes through the outer layer of the AF.

FIG. 23D is a superior view of a partially exploded embodiment of the invention and portion of the AF drawn in FIG. 23C. The cable was pulled through the aperture created by the needle, which pulls a portion of the flexible longitudinal fixation component through such aperture, then the flexible longitudinal fixation component was cut to release the cable and form two flexible longitudinal fixation components.

FIG. 23E is a lateral view of the embodiment of the invention drawn in FIG. 23A. One end of the flexible longitudinal fixation component 2310 was passed through the first and second cable components 2304′, 2304′, then such end of the flexible fixation component was welded to itself. The second end of the flexible longitudinal fixation component was passed through a third cable component 2304″ then such end was welded to itself.

FIG. 23F is a posterior view of the embodiment of the invention drawn in FIG. 23E and an IVD. The cut ends of the flexible longitudinal fixation component 2310 are seen extending through the aperture created by the needle. The second ends of the flexible fixation components and the cable components are seen extending through the fissure. The second ends of the cable loops, the ends with the ring components were passed through AF tissue on a second side of the fissure using the embodiments of the invention drawn in FIGS. 23B and 23C.

FIG. 23G is a posterior view of the embodiment of the invention and IVD drawn in FIG. 23F. The cable near the cranial side of the IVD was pulled through an aperture created by the needle then the flexible longitudinal fixation component 2310 was cut at 2311 to release the cable. A hook-like tool 2320 is seen pulling the third cable 2304″ from the IVD. The cable will pull the end of the flexible longitudinal fixation component from the IVD then the flexible longitudinal fixation component will be cut to release the cable component. The ends of the flexible longitudinal fixation components will be welded together, in a V-shaped configuration, using the previously described methods in the final step of the procedure.

FIG. 24A is a lateral view of an alternative embodiment of the invention drawn in FIG. 23E. Elastic components 2402, 2403 are seen fastened to the ends of a flexible longitudinal fixation component 2404. For example, such components may be crimped, welded, or glued to the ends of the fixation component, using the previously described adhesives or methods. The elastic components are open in the centers and have projections from the sides of the components. The openings are preferably 1 to 3 millimeters wide and 1 to 10 millimeters long. The projections are preferably 0.5 to 2 millimeters long and 0.1 to 1.0 millimeters in diameter. The material that forms the elastic component is preferably 0.2 to 1.0 millimeters in diameter. The elastic material is preferably metal, such as stainless steel, titanium, or Nitinol or plastic, such as nylon, polypropylene, or polyethylene. The distal 1 to 3 millimeters of the elastic component are preferably angled relative to the proximal portions of such elastic components. For example, the angle between such portions of the elastic component is preferably between 100 and 170 degrees.

FIG. 24B is a lateral view of a partial longitudinal cross section of the distal end of an alternative embodiment of the invention drawn in FIG. 23B and a first end of the embodiment of the invention drawn in FIG. 24A. The elastic component 2402 was inserted into the slot in the needle component 2202. The flexible longitudinal fixation component 2404 passes through a groove in the distal end of the insertion tool and is fastened to the proximal end of the insertion tool, for example using the embodiment of the invention drawn in FIG. 34. Tension, preferably between 1 and 25 pounds, or higher, on the flexible longitudinal fixation component holds the elastic component in the slot in the needle and assists in deploying the needle and/or holding the needle in the deployed position. Tension on the fixation component increases the force with which the needle is deployed, which helps the needle pass through disc material, including NP tissue.

The pointed end 2203 of the needle 2202, or the proximal end, for example the last 2 to 10 millimeters of the needle is narrower than the remaining portion of the needle. For example, the cross section of the proximal end of the needle could be generally rectangular and 0.4 to 1.0 millimeters wide and 0.5 to 2.0 millimeters long. The remaining portion of the needle could be generally circular in cross section and be approximately 0.8 to 2.0 millimeters in diameter. The narrow proximal end of the needle provides additional room for the elastic component in the tubes of the instrument when the needle is constrained.

FIG. 24C is a lateral view of the distal end of the embodiment of the invention drawn in FIG. 24B. The needle is constrained within the insertion tool. FIG. 24D is an anterior view of the distal end of the embodiment of the invention drawn in FIG. 24C. The needle is drawn in its deployed position. FIG. 24E is a superior view of the distal end of the embodiment of the invention drawn in FIG. 24D and a partial axial cross section of a portion of the AF. The deployed needle was pulled through the AF using the previously described methods, for example as described in FIG. 23B. An outer most tube was advanced distally into the deployed needle as previously described, for example in FIG. 22A.

FIG. 24F is a superior view of the first end and the portion of the AF drawn in FIG. 24E. The deployed needle was pushed into the IVD, then constrained and the insert tool pulled from the IVD. The projections 2405 from the sides of the elastic component impinged against the outer layer of the AF, which help eject the elastic component from the slot in the needle. FIG. 24G is a posterior view of the embodiment of the invention and IVD drawn in FIG. 24F.

FIG. 24H is a superior view of the embodiment of the invention drawn in FIG. 24G and an axial view of a portion of the AF. The second elastic component 2403 was passed through a surgically created aperture in the AF in a second side of the fissure using the previously described technique, for example the technique described in FIGS. 24E and 24F.

FIG. 24I is a superior view of an exploded embodiment and portion of the AF drawn in FIG. 24H. The elastic components were pulled away from the AF then the flexible longitudinal fixation component was cut to release the elastic components.

FIG. 24J is a superior view of an alternative embodiment of the invention drawn in FIG. 24I and an axial cross section of a portion of the AF. An end of the flexible longitudinal fixation was passed through an intra-aperture component 2420 then the elastic component was fastened to the end of such flexible fixation component. The elastic components were then passed through surgically created apertures in the AF using the methods described in FIGS. 24F-24H. Intra-aperture components are described in my co-pending patent applications, such as Ser. No. 12/263,753.

FIG. 24K is a superior view of the embodiment of the invention and portion of the AF drawn in FIG. 24J. The intra-aperture component 2420 was placed in the fissure, tension was applied to the ends of the flexible longitudinal fixation component 2404, then the ends of the flexible fixation components were welded to each other and the portions of the excess flexible fixation material cut and remove.

FIG. 24L is a superior view of a first end of the embodiment of the invention drawn in FIG. 24J and an axial cross section of a portion of the AF. The projections 2405 from the elastic component are seen impinging on the outer layer of the AF. High tension force on the flexible longitudinal fixation component preferably pulls the elastic component into the IVD. However the force exerted by the needle as the needle is pulled into the IVD is preferably too low to pull the elastic component into the IVD. For example, tensile forces of at least 1, 2, 3, 4, 5, 6, 7, 8, 9, or 10 pounds may be required to pull the elastic component into the IVD.

FIG. 25A is an inferior view of a portion of an alternative embodiment of the invention drawn in FIG. 24L. An elastic wire component 2504 is seen constrained in recesses of a stiffer generally rectangular second component 2502. The ends 2506 of the wire component 2504 extend beyond the sides of the generally rectangular component. The rectangular component 2502 is preferably made of metal, such as stainless steel and is preferably 0.5 to 2 millimeters wide, 1 to 6 millimeters long and 0.2 to 1 millimeter thick. The wire is preferably made of metal, such as stainless steel and is preferably 0.1 to 0.5 millimeters in diameter. The openings between the wire and the rectangular component are preferably 1 to 3 millimeters wide and 1 to 5 millimeters long.

FIG. 25B is a superior view of the embodiment of the invention drawn in FIG. 25A and a portion of a flexible longitudinal fixation component 2510. The end of the flexible longitudinal fixation component was passed between the wire and the rectangular component then welded at 2508. The second end of the wire component is placed into the slot in the needle component of the insertion tool. The ends of the wire impinge against the outer layer of the AF similar to the method demonstrated in FIG. 24L.

FIG. 26A is a superior view of an alternative embodiment of the invention drawn in FIG. 25A. The elastic wire component is similar in size to the embodiment of the invention drawn in FIG. 25A. The component 2602 is preferably made of metal such as stainless steel, titanium, Nitinol, or plastic. One end of the component preferably impinges on the outer layer of the AF after the component is passed through the AF using the previously described methods. FIG. 26B is a superior view of the embodiment of the invention drawn in FIG. 26A and a portion of a flexible longitudinal fixation component 2610 the end of which was passed through the elastic component then welded to itself.

FIG. 27A is a superior view of one end of an alternative embodiment of the invention drawn in FIG. 26B. A wire component was wrapped around the end of a flexible longitudinal fixation component. A loop of the wire component extends 1 to 10 millimeters beyond the end of the fixation component. The ends of the wire loop extend beyond the wire coil and preferably impinge on the outer layers of the AF when the component is passed through the AF. The wrapping the wire around the fixation component fastens the components together. An adhesive, such as the previously mentioned adhesives may be added to the components to help fasten them together. FIG. 27B is a superior view of one end of an alternative embodiment of the invention drawn in FIG. 27A. The coiled wire 2702′ fastens a wire loop to the end of a flexible longitudinal fixation component.

FIG. 28A is a superior view of one end of an alternative embodiment of the invention drawn in FIG. 27B. A metal or plastic component 2802 is seen fastened to an end of a flexible longitudinal fixation component 2810. For example, the two components could be crimped, swagged, swedged, and/or glued together. The elastic distal component is preferably 2 to 10 millimeters long, 0.4 to 2.0 millimeters in diameter and made of metal, such as stainless steel or plastic. One to three millimeter long projections 2805 are seen near the end of the flexible fixation component. As previously described, the ends of the projections preferably face the flexible fixation component. The distal 1 to 3 millimeters of the elastic component is angled relative to the proximal end of such component. Such angle α is preferably 15 to 85 degrees, and most preferably between 35 and 55 degrees. FIG. 28B is a lateral view of the embodiment of one end of the embodiment of the invention drawn in FIG. 28A.

FIG. 28C is a superior view of a partial longitudinal cross section of a portion of an alternative embodiment of the invention drawn in 24E and the embodiment of the invention drawn in FIG. 28A. The distal end of the elastic component is seen in a hole near the pointed end of the needle. The elastic component is released from the needle as the needled is pulled into the IVD. Impingement between the projections of the elastic component and the outer layer of the AF facilitate such release.

FIG. 29A is a superior view of one end of an alternative embodiment of the invention drawn in FIG. 28A. An elastic component 2902 fastens a U-shaped wire loop 2904 to an end of a flexible longitudinal fixation component 2910. For example, such components could be crimped or glued together using the previously described materials and techniques. Projections 2903 extend from the sides of the elastic component 2910. Such projections are preferably 0.5 to 3 millimeters long and 0.3 to 1 millimeters in diameter. The elastic component is preferably 1 to 5 millimeters long, 0.8 to 2.0 millimeters in diameter, and made of the previously mentioned elastic materials. The projections are drawn in the constrained shape, which may be seen as the elastic component is pulled through a surgically created aperture in the AF.

FIG. 29B is a superior view of the end of the embodiment of the invention drawn in FIG. 29A. The elastic component 2902 is seen its expanded shape, which may be seen after the component passes through the AF.

FIG. 29C is a superior view of one end of an alternative embodiment of the invention drawn in FIG. 29A. The elastic component 2902′ has a single projection 2903′. The elastic component is otherwise similar to size to the component described in FIG. 29A and made of similar materials. Three, four, five or more projections may be used in alternative embodiments of the invention. FIG. 29D is a superior view of the end of the embodiment of the invention drawn in FIG. 29C. The elastic component 2902′ is seen in its expanded shape. FIG. 29E is a superior view of a longitudinal cross section of the end of the embodiment of the invention drawn in FIG. 29B.

FIG. 30A is a superior view of an alternative embodiment of the invention drawn in FIG. 25A. The ends of the wire component 3004 are twisted around each other. The component is preferably similar in size to the component in FIG. 29A and preferably made of the previously described elastic materials. The ends 3003 of the wire component 3004 impinge on the outer layer of the AF after the component is passed through a surgically created aperture in the AF.

FIG. 30B is a superior view of the embodiment of the invention drawn in FIG. 30A and a portion of a flexible longitudinal fixation component 3010 an end of which was passed through the wire component then welded to the fixation component. FIG. 30C is a superior view of an alternative embodiment of the invention drawn in FIG. 30B. The wire is fastened to the flexible longitudinal fixation component using the methods described in FIGS. 27A and 27B.

FIG. 31A is a superior view of an alternative embodiment of the invention drawn in FIG. 30B. The end of a flexible longitudinal fixation component 3110 was passed through the elastic component 3102 then welded to itself. The elastic component is similar in size to the component described in FIG. 30C and is made of the previously mentioned elastic materials. For example, the component could be formed by welding the ends of a wire together. The component is shown in its constrained shape, which may be seen as the component is passed through a surgically created aperture in the AF. FIG. 31B is a superior view of the embodiment of the invention drawn in FIG. 31A. The elastic component is seen in its expanded or unconstrained shape, which may be seen after the component is pulled through an aperture in the AF.

FIG. 32A is a superior view of one end of an alternative embodiment of the invention drawn in FIG. 31A. An elastic component 3202 is seen proximal to a welded area 3203 of a flexible longitudinal fixation component 3210. The flexible longitudinal fixation loop is placed in the slot of a needle. The elastic component is crimped, welded, or glued to the flexible longitudinal fixation component. The elastic component is preferably 1 to 5 millimeters long, has a 0.3 to 0.8 millimeter inner diameter and has a 0.4 to 2.0 millimeter outer diameter. The elastic component is made of the previously described materials. The projection is seen in its constrained shape. The opening in the flexible longitudinal fixation loop is preferably at least 1 millimeter wide and 1 to 10 millimeters long. The opening in flexible longitudinal fixation loop is preferably 3 to 10 centimeters long or longer in alternative embodiments of the invention. Such long openings place the welded area of the flexible longitudinal fixation component outside the IVD as the distal end of the tool is inserted and removed from the IVD, which protects the weld.

FIG. 32B is a superior view of one end of an alternative embodiment of the invention drawn in FIG. 32A. The projection 3205 is seen in its expanded shape. The elastic component 3202 helps release the flexible fixation member from the slot in the needle by the previously described impingement on the outer layer of the AF. The elastic component also helps prevent peeling the weld apart if the flexible loop is pulled back into the IVD.

FIG. 32C is a superior view of one end of an alternative embodiment of the invention drawn in FIG. 32B. The elastic component has four projections, which are seen in their constrained shape. Two, 3, 5, 6 or more projections may be used in alternative embodiments of the invention. FIG. 32D is a superior view of one end of the embodiment of the invention drawn in FIG. 32C. The projections are seen in their expanded shape.

FIG. 32E is a superior view of one end of an alternative embodiment of the invention drawn in FIG. 32D. Knots 3212, 3214 are seen proximal to and distal to an elastic component on the flexible longitudinal fixation component. The knots are used to limit movement of the elastic component over the fixation component.

FIG. 32F is a superior view of one end of an alternative embodiment of the invention drawn in FIG. 32E. The elastic component 3222 is seen within the welded loop of the flexible fixation component 3210. Alternative components, that are wider than the diameter of the flexible longitudinal fixation component, including a knot or knots 3216 as shown in FIG. 32G, could be placed in a similar location or locations within the welded loop of the flexible fixation component in alternative embodiments of the invention.

FIG. 32G is a superior view of one end of an alternative embodiment of the invention drawn in FIG. 32F. The flexible longitudinal fixation component is knotted proximal to the weld 3218. One or knots are preferably pulled through surgically created apertures in the AF to help release the flexible fixation component from a slot in a needle. The knots also help prevent peeling the weld apart if the weld is pulled back into the IVD. One, 2, 4, 5, or more knots may be used in alternative embodiments of the invention. The distal-most knot is preferably with 2 millimeters or less of the weld.

FIG. 32H is a superior view of one end of alternative embodiments of the invention drawn in FIGS. 23E and 32G. The ends of the cable are fastened together, for example by a crimped metal sleeve, which is not seen in the drawing. A loosely tied knot 3230 is seen near one end of the cable. We discovered such loosely tied knot is compressed as it passes through the AF then give the elasticity of the cable, the knot expands once it passes through the outer layer of the AF. The stiffness of the expanded knot and the stiffness of the remaining portion of the cable external to the AF, cause the cable to slid out of the slot as the needle is pulled back into the IVD.

FIG. 33A is a superior view of an alternative embodiment of the needle-like component invention drawn in FIG. 28C. The vertical arm 3303 of the needle 3302 is generally rectangular in cross section and the angled portion 3305 of the needle is generally circular in cross section. The rectangular portion of the needle is preferably 1 to 2 millimeters wide, 0.5 to 1.5 millimeters thick and 5 to 18 millimeters long. The cylindrical portion of the needle is preferably 1 to 2 millimeters in diameter and 5 to 15 millimeters long. The end of the needle near the circular opening may be generally rectangular in alternative embodiments of the invention. The slot in the needle preferably exits into the pointed end of the needle. FIG. 33B is an anterior view of the embodiment of the invention drawn in FIG. 33A.

FIG. 33C is a lateral view of the pointed end of the embodiment of the invention drawn in FIG. 33B and a portion of one end of the embodiment of the invention drawn in FIG. 32G. The edge of the needle opposite the slot of the needle may be tapered in alternative embodiments of the invention to help the needle pass through material such as NP tissue.

FIG. 33D is a lateral view of the pointed end of an alternative embodiment of the invention drawn in FIG. 33C and a portion of one end of the embodiment of the invention drawn in FIG. 32G. The cross section of the pointed half of the needle is generally triangular in shape.

FIG. 34 is a superior view of the proximal end of an alternative embodiment of the invention drawn in FIG. 24E. A flexible longitudinal fixation component 3410 is seen cleated or otherwise releasably fastened to the proximal end of insertion tool 3420. One end of the flexible longitudinal fixation component was placed into the slot in the needle do the insertion tool. A spring 3430 compressed between the proximal end of the tubes of the insertion tool and the proximal component of the tool, which holds the flexible longitudinal fixation component maintains tension on the flexible member as the needle moves from the constrained to the deployed positions. The needle, although not shown in the drawing, is in its constrained position. The spring preferably maintains 1 to 25 pounds of tensile force, more preferably 4 to 15 pounds of tensile force for 10 to 30 millimeters of travel, as the needle moves from the constrained to the deployed position. The spring 3430 preferably travels over a rod 3440 component 3442 that extends through the component with the cleat.

FIG. 35A is a superior view of an alternative embodiment of the invention drawn in FIG. 24B and an axial cross section of a portion of the AF. A tool 3502 with an extendable loop 3504 is seen in the fissure of the AF. The extendable loop 3504 is seen in its retracted position. FIG. 35B is a superior view of the embodiment of the invention and axial cross section of the portion of the AF drawn in FIG. 35A. The loop 3504 is seen in its extended position. The cannulated sleeve component is preferably 15 to 30 centimeters long, has an outer diameter of 3 to 6 millimeters and is 0.2 to 1.0 millimeters thick. The sleeve is preferably made of metal, such as stainless steel or plastic. The extendable loop is preferably made of Nitinol or other springy elastic material such as titanium, steel, or plastic. The diameter of the material used to form the extendable loop is preferably 0.5 to 2.0 millimeters in diameter. The extended loop is preferably 5 to 15 millimeters long. The opening in the extendable loop is preferably about 1 to 8 millimeters wide and 1 to 14 millimeters long.

The proximal ends of the loop may extend to beyond the proximal end of the sleeve component or fastened to a rod or tube component within the sleeve component. For example, the ends of a 1.4 millimeter diameter 30 millimeter long Nitinol wire could be crimped in the end of a 2.8 millimeter inner diameter stainless steel tube. Such tool could be passed through a sleeve with an inner diameter between 3 and 3.2 millimeters and an outer diameter of 4 millimeters. The distal end of the sleeve is preferably angled 90 to 145 degrees relative to the shaft of the sleeve. Alternatively, the ends of the wire loop could be fastened perpendicular to the side of the distal end of a rod component. The plane of the opening at the distal end of a sleeve component used with such wire loop component could be perpendicular or inclined relative to the shaft of the sleeve, however the distal end of the shaft of such sleeve could be straight rather curved as shown in the drawing.

FIG. 35C is a lateral view of a partial cross section of the embodiment of the invention drawn in FIG. 35B. The flexible loop 3504 is seen in its extended, open position. The loop, made of elastic and/or shape memory material assumes its open position when it is released from the constraint of the sleeve and/or it changes temperature.

FIG. 35D is a superior view of the embodiments of the invention drawn in FIGS. 5A and 35C and axial cross section of the portion of the AF. The tip of the needle-like tool was advanced through AF tissue lateral to the fissure. The needle may be inserted through guide component, similar to the one drawn in FIG. 36A, which is placed over the cannulated shaft of the loop tool. The distal end of the needle could be curved in an alternative embodiment of the invention. The tips of curved needles are directed away from fissures in the AF. The vertical arms of flexible longitudinal fixation components placed through holes in the AF created by curved needles converge towards the outer layer of the AF. Such flexible longitudinal fixation components grasp more of the inner than the outer portions of the AF, which helps close the inlet of fissures in the AF.

FIG. 35E is a superior view of the embodiment of the invention and axial cross section of the portion of the AF drawn in FIG. 35D. The tip of the needle-like tool was advanced through the flexible loop. The tip of the needle is preferably inserted 10 to 20 millimeters through the outer layer of the AF.

FIG. 35F is a superior view of the embodiment of the invention and axial cross section of the portion of the AF drawn in FIG. 35E. A generally cylindrical component 3512 at the end of the flexible longitudinal fixation component 3510 or wire loop was forced from the tip of the needle and the needle-like tool was removed from the IVD. Retraction of the flexible loop pulls the flexible fixation component or wire loop component into or onto the distal end of the cannulated sleeve. FIG. 35G is a superior view of the embodiment of the invention and axial cross section of the portion of the AF drawn in FIG. 35F. The flexible longitudinal fixation component 3510 or wire loop is captured within the retracted flexible loop 3504.

FIG. 35H is a superior view of the embodiment of the invention and axial cross section of the portion of the AF drawn in FIG. 35G. The tool with the flexible loop was pulled from the IVD, which pulled the flexible longitudinal fixation component 3510 or the wire loop through the fissure. The distal end of the flexible longitudinal fixation component 3510 or the wire loop was cut to release the flexible loop tool. The fissure end of the flexible longitudinal fixation component is next passed through AF tissue on a second side of the fissure using the embodiments of the invention drawn in FIGS. 35A-H or previous embodiments of the invention.

FIG. 35I is a superior view of an alternative embodiment of the invention drawn in FIG. 35A. The loop 3520 is seen in its relatively closed position. FIG. 35J is a superior view of the embodiment of the invention drawn in FIG. 35I. Tension on a longitudinal component through the center of the instrument causes the articulating components to move together in the proximal to distal direction and apart in the lateral direction, which creates a wider opening to receive the distal end of the flexible longitudinal fixation component or cable as shown in FIG. 35E.

FIG. 35K is a lateral view of the distal end of an alternative embodiment of the invention drawn in FIG. 35C. Multiple openings 3532 are seen on the top of the distal end of the component 3530. FIG. 35L is a superior view of the embodiment of the invention drawn in FIG. 35K and an axial cross section of a portion of the AF. The tip of a needle of an instrument like the embodiment of the invention drawn in FIG. 5A was passed through one of the openings in the distal end of the tool, then the component at the distal end of the flexible longitudinal fixation component or wire loop was forced through the needle. Elastic members between the openings in the distal end of the instrument shown in this figure return to their resting positions, which reduces the width of the openings to less than the width of the component at the distal end of the flexible longitudinal fixation or wire component. The instrument is then pulled from the IVD, which pulls the end of the flexible fixation member or wire loop through the fissure.

FIG. 35M is a superior view of the distal end of an alternative embodiment of the invention drawn in FIG. 35K. The distal end 3552 of an angled clamp or grasping tool 3550 can be used to grasp the flexible longitudinal fixation component after it is passed through the AF with the needle-like instrument drawn in FIG. 5A. The arms of the clamp are preferably 3 to 6 millimeters long and about 1 millimeter wide.

FIG. 35N is a lateral view of the distal end of an alternative embodiment of the inventions drawn in FIGS. 35C & 42D. The distal ends of two elastic wire-like components 3560, 3562 are seen extending from the distal end of the outer sleeve 3566 of an insertion tool, which constricts the elastic wires. The proximal ends of the wires are fastened to the distal end of the shaft or inner tube component, which lies within the outer sleeve component. The distal ends of the wires lie within the outer sleeve when the inner sleeve is fully retracted in the outer sleeve.

FIG. 35O is a lateral view of the distal end of the embodiment of the invention drawn in FIG. 35N. The outer sleeve was fully retracted. The wires are seen in their fully deployed position, which form a loop-like opening. The released wires also bend about 90 degrees in a second plane as shown in FIG. 42F. The device is made of similar materials and is a similar size to the embodiment of the invention described in FIGS. 42D-42T. For example, the wires are preferably made of Nitinol. The opening formed by the two wires is preferably about the size of the opening of the loop feature described in FIGS. 42D-42T. The first wire is preferably longer than the second wire, which places the contact between the two wires off of the midline of the device. Such location of the contact point between the distal ends of the wires helps prevent the cable loops from passing between the wires as the distal end of the device is pulled from IVDs. The distal ends of the wires are preferably tapered to form a cutting surface and to form an area of contact between the two wires that is an a ninety degrees angle relative to the adjacent portions of the wires.

FIG. 35P is a lateral view of the distal end of an alternative embodiment of the invention drawn in FIG. 35N. The distal ends of two elastic wire-like components 3570, 3572 are seen extending from the distal end of the outer sleeve 3577 of an insertion tool, which constricts the elastic wires. The proximal ends of the wires are fastened to the distal end of the shaft or inner tube component, which lies within the outer sleeve component. The distal ends of the wires lie within the outer sleeve when the inner sleeve is fully retracted in the outer sleeve.

FIG. 35Q is a lateral view of the distal end of the embodiment of the invention drawn in FIG. 35P. The outer sleeve was fully retracted. The wires are seen in their fully deployed position, which form a loop-like opening. The distal ends of the wires overlap or cross across each other. The wires may be of the same or different length. The released wires also bend about 90 degrees in a second plane as shown in FIG. 42F. The device is made of similar materials and is a similar size to the embodiment of the invention described in FIGS. 42D-42T.

FIG. 35R is a lateral view of the distal end of an alternative embodiment of the invention drawn in FIG. 35Q. The outer sleeve 3588 was fully retracted. The wires 3580, 3582 are seen in their fully deployed position, which form a loop-like opening. A joint connects the distal ends of the wires. For example, an axle 3590 may pass through holes in the distal portions of the wires. The wires are preferably the about the same length. The released wires also bend about 90 degrees in a second plane as shown in FIG. 42F. The proximal ends of the wires are fastened to the distal end of the shaft or inner tube component, which lies within the outer sleeve component. A joint may also connect the proximal ends of the wires within the shaft of the insertion component or just distal to the distal end of the insertion component. The distal ends of the wires lie within the outer sleeve when they the inner sleeve is fully retracted in the outer sleeve. The device is made of similar materials and is a similar size to the embodiment of the invention described in FIGS. 42D-42T.

FIG. 35S is a lateral view of the distal end of an alternative embodiment of the invention drawn in FIGS. 35R and 40C. The outer sleeve was fully retracted. The wire is seen in its fully deployed position, which forms a loop-like opening. The wire preferably completes at least 270 degrees of a circle or oval. The distal end of the fully deployed wire most preferably contacts the distal end of the shaft of the insertion component. The released wire also bends about 90 degrees in a second plane as shown in FIG. 42F. The device is made of similar materials and is a similar size to the embodiment of the invention described in FIGS. 42D-42T.

FIG. 36A is a superior view of an alternative embodiment of the invention drawn in 35L and an axial cross section of a portion of the AF. The needle 3602 of an instrument such as drawn in FIG. 5A or similar to a “clothes tagging gun” (Premier Packaging, Macedon, N.Y.) is seen passing through the AF and the opening of a flexible loop 3604. The needle is also passed through a guide component 3610 that was placed over the sleeve through which the flexible loop tool was inserted. The guide component slides along the sleeve component but the guide component is unable to rotate about the longitudinal axis of the sleeve. For example, a pin from the sleeve component could pass through a slot in guide component to enable longitudinal motion between the components but prevent axial rotation between such components. The smaller diameter opening of the guide component, through which the needle lies over the opening in the flexible loop component

FIG. 36B is a superior view of the embodiment of the invention an axial cross section of the portion of the AF drawn in FIG. 36A. Projections 3612, 3614 from the sides of the distal end of the flexible longitudinal component 3610 rotate from the longitudinal component as the flexible longitudinal component is forced from the needle. The distal end of the flexible longitudinal component is preferably wider than the opening in the flexible loop component. The flexible loop component is pulled through the fissure, with or without retraction of the flexible loop to pull the end of the longitudinal fixation component through the fissure. A flexible longitudinal fixation component is fastened to the proximal end of the flexible longitudinal component (not shown in the drawing). The transverse portion of the distal end of the flexible longitudinal component is preferably 4 to 15 millimeters wide and 0.7 to 2 millimeters in diameter. The longitudinal portion of such component is preferably 20 to 100 millimeters long and 0.7 to 2 millimeters in diameter. Such component is preferably made of plastic, such as nylon, polyethylene, or polypropylene or metal such as stainless steel, Nitinol, or titanium.

FIG. 37A is a superior view of the distal end of an alternative embodiment of the invention drawn in FIG. 36B. The distal end of the flexible longitudinal component 3710 is fastened to a separate transverse component 3712. The transverse component is preferably similar in size to the transverse component drawn in FIG. 36B and preferably made of similar materials to the transverse component drawn in FIG. 36B. The distal end of the flexible longitudinal component was passed through a hole in the transverse component then a malleable component 3714 was crimped to the end of the flexible longitudinal component. The longitudinal axis of the transverse component at the distal end of the flexible longitudinal component is collinear and preferably coaxial with the longitudinal axis of the needle component, when such component is within the lumen in the needle component. The transverse component is preferably pushed 1 to 5 millimeters beyond the tip of the needle just after the tip of the needle is pushed beyond the inner layer of the AF.

The needle, with the transverse component, is then advanced to the inner layer of the anterior portion of the AF followed by retraction of the needle component while applying pressure on the proximal end of the transverse component with the stylet. The distal end of the transverse component is thus ejected into or near the inner layers of the anterior portion of the AF. Partial expulsion of the transverse component from the needle just after the tip of the needle is pushed beyond the inner layers of the posterior AF prevents the needle from injuring the anterior AF. For example, the distal end of the transverse component could be pushed 1 to 5 millimeters, or more beyond the point of the needle, after the needle is inserted 5 to 12 millimeters into the IVD. The needle-like tool preferably has a mechanism that initially locks the stylet after the stylet is advanced 1 to 5 millimeters relative to the shaft of the needle, then allows retraction of the needle relative to the stylet or advancement of the stylet relative to the needle, to expulse the transverse component from the needle.

FIG. 37B is a superior view of an alternative embodiment of the invention drawn in FIG. 37A. A end of a wire cable component was passed through a hole in the transverse component 3722 then fastened to the other end of the wire cable, for example with a crimp, to form a cable loop. The transverse component is somewhat like a bead on a wire necklace.

FIG. 37C is a superior view of an alternative embodiment of the invention drawn in FIG. 37B. The end or ends of a wire cable, or a flexible longitudinal fixation component 3730 are seen welded or glued to the transverse component 3732. The previously mentioned adhesives can be used to glue such components or the components could be laser welded together.

FIG. 37D is a superior view of an alternative embodiment of the invention drawn in FIG. 37C. A generally cylindrical component 3742 with an elastic projection 3744 is seen crimped to the end of a flexible longitudinal fixation component or a wire cable 3740.

FIG. 38A is a superior view of an alternative embodiment of the invention drawn in FIG. 37D and an axial cross section of a portion of the AF. The distal end of a flexible longitudinal component 3802 was passed through the AF with a needle-like tool. The proximal end of the flexible longitudinal component is bonded to a transverse component 3810. FIG. 38B is a superior view of the embodiment of the invention and axial cross section of the portion of the AF drawn in FIG. 38A. Tension on the proximal end of the flexible longitudinal component pulls the end of the transverse component 3810 against the inner layer of the AF, which rotates the transverse component such than the longitudinal axis of the transverse component is generally parallel with the inner layer of the AF. The end of the transverse component is seen extending into the fissure 3820. The end of the flexible longitudinal component is preferably fastened to near one end of the transverse component. The transverse component is preferably 6 to 15 millimeters long and 0.6 to 2 millimeters in diameter. The transverse component 3810 is preferably made of the previously listed metal or plastic materials. The end of the transverse component 3810 is grasped with an instrument passed through the fissure in the next step of the procedure.

FIG. 38C is a lateral view of the embodiment of the invention drawn in FIG. 38B and a sagittal cross section of a portion of the spine. The ends of the transverse component preferably impinge against the vertebral endplates 3830, 3832 as illustrated in the drawing. Alternatively, only one end of the transverse component may impinge against a single vertebral endplate. FIG. 38D is a superior view of an alternative embodiment of the invention drawn in FIG. 38B and an axial cross section of a portion of the AF. The transverse component, if made of a shape memory material such as Nitinol, is shown in its second shape, after it is released from the needle. Such shape facilitates passage of the transverse component through the fissure.

FIG. 38E is a superior view of an alternative embodiment of the invention drawn in FIG. 38D and an axial cross section of a portion of the AF. The drawing illustrates an alternative second shape of the transverse component drawn in FIG. 38D. FIG. 38F is a superior view of an alternative embodiment of the invention drawn in FIG. 38E and an axial cross section of a portion of the AF. The drawing illustrates an alternative second shape of the transverse component drawn in FIG. 38E. FIG. 38G is a superior view of an alternative embodiment of the invention drawn in FIG. 38F and an axial cross section of a portion of the AF. The drawing illustrates an alternative second shape of the transverse component drawn in FIG. 38F. FIG. 38H is a superior view of an alternative embodiment of the invention drawn in FIG. 38G and an axial cross section of a portion of the AF. The drawing illustrates an alternative second shape of the transverse component drawn in FIG. 38G.

FIG. 38I is a superior view of the embodiment of the invention and axial cross section of the portion of the AF drawn in FIG. 38H. An elastic component 3840, seen in its second shape is seen extending from a sleeve 3842. The distal end of such elastic component is pushed from the sleeve, into the IVD and lateral to the lateral end of the transverse component. The hook-like end 3841 of the elastic component pulls the transverse component into the fissure as the elastic component is pulled back into the sleeve. The elastic component is preferably made of Nitinol or other shape-memory material.

FIG. 39A is a superior view of the distal end of an alternative embodiment of the invention drawn in FIG. 36A. Elastic projections 3904 extend from the sides of the distal end of the component 3902. The elastic projections are preferably 1 to 7 millimeters long and 0.1 to 1.0 millimeters in diameter. Such elastic projections are preferably made of one of the previously listed metals or plastics. The elastic projections extend from a flexible longitudinal component 3902 that is preferably 1 to 3 millimeters in diameter and 15 to 30 centimeters long. The flexible longitudinal component 3902 is preferably made from one of the previously listed metals or plastics. FIG. 39B is a lateral view of the distal end of the embodiment of the invention drawn in FIG. 39A.

FIG. 39C is a superior view of the distal end of the embodiment of the invention drawn in FIG. 39B and an axial cross section of an IVD. The distal end of a flexible longitudinal fixation component or wire loop 3910 was passed through a needle like instrument and placed near the anterior AF 3920. A sleeve 3922 was passed through a fissure in the AF. The distal end of the embodiment of the invention drawn in FIG. 39A is seen extending from the distal end of the sleeve.

FIG. 39D is a superior view of the distal end of the embodiment of the invention and axial cross section of the IVD drawn in FIG. 39C. The embodiment of the invention drawn in FIG. 39A was advanced into or just beyond the flexible longitudinal component then rotated to capture the flexible longitudinal component. The embodiment of the invention drawn in FIG. 39A is pulled through the fissure in the next step of the procedure, which pulls the flexible longitudinal component through the fissure.

FIG. 40A is a superior view of an alternative embodiment of the invention drawn in FIG. 39A. The distal end of an elastic longitudinal member 4002, which is preferably hook-shaped is seen extending from the distal end of a sleeve 4004, which may be curved as shown. FIG. 40B is a superior view of the embodiment of the invention drawn in FIG. 40A and an axial cross section of an IVD. The distal end of a wire loop 4010 was placed near the anterior AF 4012 using a needle-like tool 4020. The embodiment of the invention drawn in FIG. 40A was inserted through a fissure in the AF. FIG. 40C is a superior view of the embodiment of the invention and axial cross section of the IVD drawn in FIG. 40B. The distal end of the elastic longitudinal component was advanced beyond the wire loop component.

FIG. 40D is a superior view of the embodiment of the invention and axial cross section of the IVD drawn in FIG. 40C. The elastic longitudinal component was rotated about 90 degrees in the axial plane then pulled into the sleeve. The wire loop becomes captured between the hook and the sleeve as the proximal end of the elastic longitudinal component is pulled away from the sleeve. A portion of the flexible longitudinal fixation component is pulled through the fissure as the elastic longitudinal component is pulled from the fissure. The elastic component is preferably made of Nitinol, stainless steel, titanium or plastic. The wire-like component is preferably 1 to 2.5 millimeters in diameter and 15 to 30 centimeters long. The hook-like portion of the component is preferably 2 to 8 millimeters wide and 1 to 6 millimeters long.

FIG. 41A is a lateral view of the distal end of an alternative embodiment of the invention drawn in FIG. 40A. The distal end 4104 of the flexible longitudinal component 4102 is seen in its constrained or first shape. A sleeve 4108 is used to hold the elastic flexible component is such shape. The flexible longitudinal component is preferably made of an elastic material such as stainless steel, titanium, plastic or most preferably Nitinol. The flexible component 4102 is preferably manufactured by a 5 to 15 millimeter long longitudinal cut into the distal end of a wire that is 1 to 3.5 millimeters in diameter and 15 to 30 centimeters long. FIG. 41B is a lateral view of the distal end of the embodiment of the invention drawn in FIG. 41A. The elastic component is seen its second, unconstrained or second shape memory shape.

FIG. 41C is a superior view of the distal end of the embodiment of the invention drawn in FIG. 41B and an axial cross section of a portion of the AF. The flexible longitudinal fixation component or wire cable 4110 was passed through the AF with a needle-like tool. The elastic embodiment of the invention of FIG. 41A was pushed beyond the flexible longitudinal fixation component or wire cable component after the sleeve was passed through the fissure. Axial rotation of the wire or elastic longitudinal fixation component wraps the flexible longitudinal fixation or wire loop component around the arms at the distal end of the elastic component. The elastic component is pulled into the sleeve and the sleeve is removed from the IVD, which pulls the flexible longitudinal fixation or wire loop component through the fissure, in the next step of the procedure.

FIG. 41D is a lateral view of the distal end of an alternative embodiment of the invention drawn in FIG. 41B. Two longitudinal cuts in the distal end of the elastic component creates four arms. Three, five, six or more arms could be created in alternative embodiments of the invention. One or more components of this or other embodiments of the invention could be made of magnets. For example, the elastic longitudinal component could be made of magnetic material. FIG. 41E is an anterior view of the distal end of an alternative embodiment of the invention drawn in FIG. 41A. The distal end of the flexible longitudinal component is seen in its first or restrained shape.

FIG. 41F is a superior view of a longitudinal cross section of the distal end of the embodiment of the invention drawn in FIG. 41E. The distal end of the flexible longitudinal component is seen in its expanded shape. The sides of the arms 4120, 4122 at the distal end of the flexible longitudinal component may have grooves 4124 or other features to increase the roughness of the surface of the arms. The proximal end of the flexible component is seen pinned at 4130 to the distal end of a rod 4132. A plug 4140 is seen pinned at 4142 to the distal end of a sleeve or tube component 4144. The flexible longitudinal component is preferably made of Nitinol, has a diameter between 0.8 and 3 millimeters, and is a length between 10 and 35 millimeters long. For example, such component may preferably have a 1.5 millimeter diameter and a 25 millimeter length.

The arms of the flexible longitudinal component are preferably between 1 and 10 millimeters long. The angle between the arms is preferably between 40 and 100 degrees. For example, the arms could be 4 millimeters long and the angle between the expanded arms could be 60 degrees. In alternative embodiments of the invention the flexible longitudinal component could made of alternative elastic material, made longer or shorter, have 1, 3, 4 or more arms, have longer or shorter arms, and have a different angle than previously described. The rod is preferably between 2 and 4 millimeters in diameter, between 15 and 30 centimeters long, and made of metal such as stainless steel or made of plastic. The inner diameter of the sleeve component is slightly larger than the diameter of the rod. The sleeve is preferably 5 to 30 millimeters shorter than the rod. The plug is preferably about the same diameter as the rod and is machined with the opening illustrated. The components may be fastened with pins, as illustrated, laser welds, threads, or adhesives.

FIG. 41G is a superior view of a longitudinal cross section of the distal end of the embodiment of the invention drawn in FIG. 41F. The rod 4132 was pulled proximally about 5 to 15 millimeters relative to the sleeve, which pulls distal end of the flexible longitudinal component inside the plug. The plug and sleeve constrain the arms of the flexible longitudinal component in the position seen in the drawing. For example, the rod could be pulled 7 millimeters in a proximal direction, relative to the sleeve to constrain the arms of the flexible longitudinal component.

FIG. 42A is a lateral view of a partial transverse cross section of an alternative embodiment of the invention drawn in FIG. 35C. A wire 4202, preferably made of Nitinol, is seen in its second or expanded shape. A generally circular opening 4203 is seen in the expanded wire. Such opening is preferably 2 to 8 millimeters in diameter. The segment of wire between the circular opening and the sleeve component is preferably 1 to 10 millimeters long. The ends of the wire are fastened to a rod component 4204, which is seen within a tube or sleeve component 4206.

FIG. 42B is a lateral view of a longitudinal cross section of the distal end of the embodiment of the invention drawn in FIG. 42A. The wire is seen in its constrained shape inside the sleeve. The rod and sleeve components are similar in size and made of similar materials to such components described in FIG. 41F. The wire is preferably 0.8 to 2.5 millimeters in diameter and 15 to 35 millimeters long. The wire is preferably pinned, glued, or welded to a hole or holes in the side of the distal end of the rod.

FIG. 42C is a superior view of the distal end of the embodiment of the invention drawn in FIG. 42B. The rod was pushed 2 to 15 millimeters distally relative to the sleeve component, which allows the wire to assume the expanded shape.

FIG. 42D is a lateral view of an alternative embodiment of the invention drawn in FIG. 42A. Portions of the expanded Nitinol loop are seen distal the guide component. The needle-like tool, which inserts the cable loop and metal cylinder, is inserted through the hole in the guide shown at the top of the drawing. The diameter of such hole is preferably 0.8 to 2.5 millimeters in diameter. The ends of the Nitinol wire are crimped or otherwise fastened in the end of a tube, which is seen in a sleeve-like tool, which is seen in the larger hole in the guide component. The larger hole in the guide component is preferably 2 to 6 millimeters in diameter.

A pin 4210 is seen extending from the guide component into a slot in the sleeve component. The expanded Nitinol loop is similar in size and shape to the Nitinol loop described in FIG. 42A. The tube that holds the end of the Nitinol wire has an inner diameter between 1 and 4 millimeters and an outer diameter about 0.4 to 2 millimeters larger than the inner diameter. The inner diameter of the restraining sleeve is slightly larger than the outer diameter of the tube that holds the ends of the Nitinol wire. The outer diameter of the restraining tube/sleeve is about 0.4 to 2 millimeters larger than the inner diameter of such component and slightly smaller than the diameter of the larger hole in the guide component. The width and length of the guide component should be as small as possible, to optimize surgeon's views of the disc. The guide, restraining sleeve/tube, and the tube that holds the Nitinol wire are about 15 to 30 centimeters long. The tubes and pin are preferably made of metal, such as stainless steel. The guide component is preferably made of plastic.

FIG. 42E is a superior view of a longitudinal cross section of the embodiment of the invention drawn in FIG. 42D and an axial cross section of a portion of the AF. The distal end of the restraining sleeve is seen within a fissure in the AF. The Nitinol, or other elastic material wire 4202, is seen in its restrained or first shape.

FIG. 42F is a superior view of a longitudinal cross section of the embodiment of the invention and axial cross section of the portion of the AF drawn in FIG. 42E. The tube that holds the Nitinol wire was pushed distally relative to the restraining sleeve, which advanced the Nitinol wire into the IVD. The released Nitinol wire is seen in its second shape. The Nitinol wire passes through the NP as it assumes its second shape. A needle 4220 was advanced through the circular opening in the expanded wire. The rod will push the metal cylinder from the end of the needle into the disc in the next step of the procedure. A flexible longitudinal fixation component 4222 is seen in the proximal end of the cable loop near the top of the drawing. The needle is pulled from the disc after releasing the metal cylinder, then the Nitinol wire loop is pulled from the disc. As long as the metal cylinder is inserted at least about 15 mm beyond the Nitinol wire loop, extraction of the Nitinol wire loop pulls the metal cylinder through the fissure and a few millimeters outside the disc, without requiring the Nitinol wire to be pulled into the sleeve.

The metal cylinder could be grasped with another instrument once it is pulled outside the disc. Alternatively, the Nitinol wire could be pulled inside the sleeve to capture the cable loop, then the cable loop pulled from the disc. Such alternative technique requires pulling the Nitinol wire loop into the sleeve but eliminates the need to use the grasping instrument. The needle is preferably inserted at least 12 millimeters into the disc. More preferably the needle is inserted 15 to 25 millimeters into the disc. Temperature change rather than release of restrain could cause the shape memory wire may change from a first, constricted, shape to a second, expanded shape in alternative embodiments of the invention. The Nitinol wire may be pushed through fissures in the disc without the restraining sleeve in alternative embodiments of the invention.

FIG. 42G is a superior view of the distal end of an alternative embodiment of the invention drawn in FIG. 42F. A projection 4230 from the distal end of a tube 4232 holds the distal end of the wire loop against the shaft of the tube that holds the ends of the wire loop. FIG. 42H is a lateral view of the distal end of the embodiment of the invention drawn in FIG. 42G.

FIG. 42I is a superior view of the distal end of the embodiment of the invention drawn in FIG. 42H. The outer tube is pulled in a proximal direction relative to the tube that holds the wire loop, to release the wire loop. The distal end of the outer tube is held on or near the outside of the AF as the wire loop is pushed through the fissure. Alternatively, the distal end of the sleeve could be inserted into the fissure. In this embodiment of the invention, the distal end of the sleeve protects the AF as the wire loop changes its shape. The distal end of the sleeve is preferably 1 to 6 millimeters wide, 1 to 12 millimeters long, and 0.3 to 1 millimeter thick. Such portion of the sleeve is most preferably about 3 to 4 millimeters wide, 7 to 10 millimeters long and about 0.4 millimeters thick. The wire loop assumes the position seen in the drawing, after the loop passes through the fissure. The outer diameter of the sleeve could be 3 to 10 millimeters. The larger hole in the guide component is slightly larger than the outer diameter of the sleeve component.

In an alternative embodiment of the invention, tension on a flexible longitudinal component releasably connected to the distal end of the wire loop could pull the distal end of the wire loop in a proximal direction towards the shaft of the tube that holds the ends of the wire loop as the wire loop is pushed through the fissure. Release of the flexible longitudinal component, after the wire loop is inserted into the IVD, allows the distal end of the wire loop to bend away from the shaft of the instrument. Tension on one end of the flexible longitudinal component pulls such component from the IVD after release of the wire loop. The wire loop preferably narrows as it is pushed through the fissures in the AF.

FIG. 42J is a superior view of the distal end of the embodiment of the invention drawn in FIG. 42G and an axial cross section of a portion of the AF. The projection 4230 from the distal end of the outer tube preferably holds the distal end of the wire loop near the shaft of the inner tube, which hold the proximal ends of the wire loop. The angle between the longitudinal axis of the wire loop and the inner tube is preferably less than ninety degrees as the wire loop is pushed through the fissure in the AF. Such angle is more preferably less than 45 degrees and most preferably less than about 20 degrees as the wire loop is pushed through the fissure in the AF. Pressure from the sides of the fissure may reduce such angle as the wire loop is pushed through the fissure.

The wire is preferably made of elastic metal, such as Nitinol or heat-treated stainless steel. The wire is preferably 0.3 to 2.0 millimeters in diameter. The wire is more preferably 0.4 to 1 millimeter in diameter. The wire is most preferably 0.5 to 0.8 millimeters in diameter. The inner width of the opening through the wire loop is preferably 2 to 10 millimeters wide. The inner width of the opening though the wire loop is more preferably 3 to 8 millimeters wide. The inner width of the opening through the wire loop is most preferably 4 to 6 millimeters wide. Devices, with different sizes loops are preferably supplied to hospitals. For example, a small device with a loop whose internal diameter measures about 2 to 3 millimeters, a medium device with a loop whose internal diameter measures about 4 to 6 millimeters, and a large device with a loop whose internal diameter measures about 6 to 10 millimeters could be supplied to hospitals.

The length of wire loop, not including the ends of the wire than are fastened in the distal end of the inner tube, is preferably about the same as the width of the loop. Alternatively, such length of the wire loop could be longer or shorter then the width of the loop. Such alternative loops may be teardrop shaped. The distal end of the tube that holds of the ends of the wire is preferably oval in cross section. More proximal portions of such tube may be oval, circular or other shape in cross section. The width of the opening in the distal end of the tube is preferably about the width of the two ends of the wire, which are fastened to or inside the tube. For example, the width of such opening could be about 0.8 to 4 millimeters and more preferably about 1 to 2 millimeters.

The ends of the wire could be press fit, glued, or pinned into the opening in the distal end of the tube. Alternatively, the distal end of the tube could be crimped after the ends of the wire are inserted in the tube. Alternative fastening methods could be used in alternative embodiments of the invention. Alternatively the ends of the wire could be fastened within a hole in the distal end of a rod. The outer diameter of the tube to which wire is fastened is preferably 0.6 to 1.0 millimeters wider than the inner diameter of such tube. The outer diameter of the rod used in hold the wire in alternative embodiments of the invention is about the same size as the outer diameter of the tube used to hold the wire. The projection from the distal end of the tube is preferably about 1 to 15 millimeters long. Such projection is more preferably about 5 to 10 millimeters long. Such projection is preferably about 1 to 5 millimeters wide and more preferably about 2 to 3 millimeters wide. Such position of the wire loop, in which the wire loop is bent in a proximal direction, may be preferably used in other embodiments of the invention, such as FIG. 42B.

FIG. 42K is a superior view of the distal end of the embodiment of the invention drawn in FIG. 42J and an axial cross section of a portion of the AF. The wire loop was pushed through the fissure, which the distal end of the elastic moved away from the shaft of the inner tube. The angle between the longitudinal axes of the wire loop and the inner tube increases when the wire is pushed through the fissure. Such angle is preferably between 25 degrees and 90 degrees.

FIG. 42L is a superior view of the distal end of the embodiment of the invention drawn in FIG. 42K and an axial cross section of a portion of the AF. The angle between the longitudinal axes of the wire loop and the inner tube is larger than such angle in FIG. 42K. Such increase in angle may be caused by release of the constrained elastic wire loop or by release of the constrained loop and pulling the inner tube in a proximal direction. The pressure from release of the wire loop or the pressure from release of the wire loop and pulling the ends of the wire in a proximal direction, force the distal end of the wire loop along the inner layer or layers of the AF, which creates a small space between the inner AF and the NP.

FIG. 42M is a superior view of the distal end of the embodiment of the invention drawn in FIG. 42L and an axial cross section of a portion of the AF. The wire loop is seen in its preferred location and shape along the inner AF. The force required to position the wire in such location can be generated by release of the constrained wire, by a temperature change, by pulling the ends of the wire in a proximal direction, or by more than one of such mechanisms.

FIG. 42N is a superior view of the distal end of the embodiment of the invention drawn in FIG. 42M and an axial cross section of an intervertebral disc. A cable loop 4240 was passed through the wire loop using the embodiment of the invention described in FIG. 42F. Elastic projections 4242 are seen from the sides of the cylinder component 4244 on the distal end of the wire loop. Such projections help hold the cylinder component in the disc, preferably the NP of the disc, as the needle, through which the cylinder component was ejected, is pulled from the TVD. Tension on the proximal end of the cable loop pulls the projections through the NP then the fissure in the AF. The cylinder component is preferably about 0.8 to 1.6 millimeters in diameter and about 1 to 5 millimeters in length. Such component is more preferably 1 to 1.2 millimeters in diameter and about 3 to 4 millimeters in length. Such component preferably has 1 to 4 projections that are about 1 to 2 millimeters in length and about 0.1 to 0.4 millimeters in width and in thickness. Such component is preferably made of elastic material, such as stainless steel, nitinal, plastic, or other polymer material.

FIG. 42O is a superior view of the distal end of the embodiment of the invention drawn in FIG. 42N and an axial cross section of a portion of the AF. The distal end of a second cable loop was passed through the wire loop then the projection from the distal end of the outer sleeve was pushed into the fissure, which bends the wire loop away from the outer tube. The projection increases the angle between the longitudinal axes of the wire loop and the inner tube. Pulling the inner tube in a proximal direction relative to the outer tube increases such angle, which decreases the force on the AF as the wire loop is pulled from the IVD. Such mechanism preferably increases the angle between the longitudinal axes of the wire loop and the inner tube to 91 to 180 degrees. Such mechanism more preferably increases such angle to 130 to 180 degrees and most preferably to 150 to 180 degrees.

The wire loop is pulled from the IVD in the next step in the technique, which pulls one end of the flexible longitudinal fixation component through the fissure. The distal end of the wire loop is then placed near the inner tube and the projection from the inner tube used to hold the wire loop in such position, as shown in FIG. 42J, to use the tool to pass one or more cable loops through the AF tissue in on the opposite side of the fissure using the previous embodiment of the invention, such as FIGS. 12A-H. Alternatively, the ends of one or more flexible longitudinal fixation components pulled into the fissure could be passed through the distal ends of one or more cable loops pulled into the fissure the tension on the proximal ends of the cable loops pulls such ends of the flexible longitudinal fixation components through the surgically created aperture or apertures in the AF on the second side of the fissure. The guide component similar to the guide component shown in FIGS. 42D-F, not shown in the FIG. 42O, could have two smaller holes through which the needles pass per guide. The longitudinal axes of such holes are preferably 1 to 4 millimeters apart. Such holes may converge slightly in a proximal to distal direction to facilitate passing the needles through the wire loop.

FIG. 42P is a lateral view of the distal end of the embodiment of the invention drawn in FIG. 42J and a portion of the AF. FIG. 42Q is a lateral view of the distal end of the embodiment of the invention drawn in FIG. 42P and a portion of the AF. The inner tube was pushed into the fissure. Pressure from the AF tissue on the sides of the fissure compresses the wire loop. FIG. 42R is a lateral view of the distal end of the embodiment of the invention drawn in FIG. 42Q and a portion of the AF. The inner tube was pushed through the fissure. FIG. 42S is a lateral view of the distal end of the embodiment of the invention drawn in FIG. 42R and a portion of the AF. The wire loop was pushed through the fissure. The elastic wire expanded as it was released from the constraint of the anulus tissue. The distal end of the wire loop is seen touching the inner layers of the AF as drawn in FIG. 42L. FIG. 42T is a lateral view of the distal end of the embodiment of the invention drawn in FIG. 42S and a portion of the AF. The inner tube was pulled in a proximal direction relative to the position seen in FIG. 42S. The wire loop is in the position seen in FIG. 42M. FIG. 42U is a superior view of the distal end of the embodiment of the invention drawn in FIG. 42J. Two wire loops are seen extending from the distal end of the inner tube. FIG. 42V is a superior view of the distal end of the embodiment of the invention drawn in FIG. 42U and an axial cross section of a portion of the AF.

FIG. 42W is a superior view of a longitudinal cross section of an alternative embodiment of the invention drawn in FIG. 42F and an axial cross section of a portion of the AF. The ends of the elastic wire component are seen fastened within the distal end of an inner tube component. The elastic component is preferably made of Nitinol. The wire component is seen in its second shape after release of constraint by the outer tube or by a change in temperature. The angle between the longitudinal axes of the released portion of the wire component and the inner tube is preferably between the 90 degrees and 160 degrees. Such angle is more preferably between 90 degrees and 140 degrees. Such angle is most preferably between 90 degrees and 110 degrees. Such angel can be less than 90 degrees or more than 160 degrees in alternative embodiments of the invention.

When the wire loop is in its second shape shown in this figure, the portions of the wire within the outer sleeve but distal to the inner sleeve is preferably between 2 millimeters and 60 millimeters long. Such portions of the wire component are more preferably between 10 millimeters and 40 millimeters long. Such portions of the wire component are most preferably about 15 millimeters to 30 millimeters long. Such portions of the wire can be shorter than 2 millimeters or longer than 60 millimeters in alternative embodiments of the invention. Such portions of the wire component, which are longer than the portions of the wire drawn in FIG. 42F reduce the stress on the sharp angle of the wire loop where it exits the outer tube. The components of this embodiment of the invention are otherwise similar in size to the components described in FIG. 42F and the components in this embodiment of the invention are preferably made of the materials described in previous embodiments of the invention including FIG. 42F.

Two cable loops can be placed in the opening in the wire loop, using the methods drawn in FIG. 42O then the wire loop could be at least partially retracted into the outer sleeve followed by rotating the entire device 180 degrees. Two additional cable loops could be passed through the AF tissue on the second side of the fissure then through the wire loop followed by retraction then extraction of the wire loop from the IVD, which pulls all four cables loops through the fissure. The cable loops preferably pull the ends of the flexible longitudinal fixation components through the AF tissue on either side of the fissure then welded using the previously described technique.

FIG. 42X is a superior view of an alternative embodiment of the invention drawn in FIG. 42N. The ends of the cable loop 4260 were threaded through a hole 4262 in a small metal tube 4264 then threaded through one end of the tube. The half of the tube through which the cable passes was crimped to fasten the cable to the cylinder. The end of a flexible longitudinal fixation component is then passed through the opening in the cable loop followed by insertion of the cable loop into the IVD as shown in FIG. 42N. The longitudinal axis of the cable loop rotates about 90 degrees within the IVD after the metal cylinder is ejected from the needle, which helps hold the cylinder within the NP as the cable is manipulated. For example, tension on the cable loop from manipulation of the loop could cause the cylinder component to rotate about 180 degrees within the NP. Alternatively, the hold in the side of the cylinder could be closer to one end of the cylinder than the other end of the cylinder. For example, such hole could be 1 millimeter from the end of a 4 millimeter cylinder. The ends of the cable would preferably pass through the 3 millimeter side of the tube, which increases the area of the tube that can be crimped to hold the ends of the cable. Such cylinder rotates more than or less than 90 degrees within the IVD, which may facilitate extraction of the cylinder from the IVD. Similar to the embodiment of the invention drawn in FIG. 42N, the cylinder can be pulled from the IVD with preferably about 1 to 6 pounds of tension. The cylinder can be pulled from the disc with less than 1 or more than 6 pounds in alternative embodiments of the invention. The cylinder component is preferably 2 to 5 millimeters long and the outer diameter of such component is preferably about to 1 to 1.5 millimeters. The cylinder is preferably made of metal, such as stainless steel.

FIG. 42Y is a view of the distal end of an alternative embodiment of the invention drawn in FIG. 42D. The longitudinal axis of the hole 4272 through which the needle component is inserted is preferably different than the longitudinal axis of the hole 4270 in the loop component. The distal end of the needle generally has a single bevel. Such needle drifts in a direction away from the bevel as the needle is pushed through the AF. Thus, if the hole in the guide component and the hole in the loop component are coaxial, the needle will drift away from the center of the hole in the loop as the needle is pushed through the tough AF, which is generally 5 to 10 millimeters thick. The bevel of the needle is preferably pointed towards the closest portion of the inside of the wire loop before the needle is pushed through the AF. The needle preferably drifts towards the center of the wire loop as the needle is pushed through the AF. The ends of the wire cross before they enter the distal end of the tube to which they are fastened.

FIG. 43 is a superior view of the distal end of an alternative embodiment of the invention drawn in FIG. 41D. A metal, generally spherical component 4304 is seen at the end of a flexible longitudinal component 4306, which is connected to a longitudinal elastic component 4302. Centrifugal force generated by quick axial rotation of the elastic longitudinal component forces the metal generally spherical portion away from the elastic longitudinal component. Such rotation after the distal end of the elastic component is passed by a flexible longitudinal fixation or wire loop component that has been placed into an IVD, may be used to grab the flexible longitudinal fixation or wire loop component.

FIG. 44A is superior view of the distal end of an alternative embodiment of the invention drawn in FIG. 24B and an axial cross section of a portion of the AF. A sleeve 4402 is seen over the outer tube of the embodiment of the invention drawn in FIG. 24B. A projection 4404 extends from the side of the distal end of the sleeve. The sleeve is preferably made metal, such as stainless steel, or plastic. The inner diameter of the sleeve is slightly larger than the outer diameter of the tube 4406 inside the sleeve. The projection preferably extends 3 to 10 millimeters lateral to the side of the sleeve, just proximal to the projection. The projection is preferably 1 to 12 millimeters tall. The sleeve is shorter than the tube inside the sleeve and the sleeve advanced distally on to the outer layer of the AF after the end of the instrument is passed through the fissure. The projection indicates where the needle will penetrate the AF and protects the surrounding structures, such as nerves, from the needle. FIG. 44B is a lateral view of the embodiment of the sleeve drawn in FIG. 44A. The projection 4404 is preferably 1 to 8 millimeters wide.

FIG. 44C is a superior view of the distal end of an alternative embodiment of the invention drawn in FIG. 44A and an axial cross section of a portion of the AF. Projections 4410, 4412 are seen on the proximal end of the projection 4404 from the sleeve 4402. The elastic projection on the cable loop was pulled proximal the projections on the proximal end of the projection from the sleeve.

FIG. 44D is a lateral view of the embodiment of the invention drawn in FIG. 44C. The projections 4410, 4412 are seen on the proximal end of the projection from the sleeve. One, 2, 4, 5, 6, 7, or more such projections may be used in alternative embodiments of the invention. The projections are preferably 0.5 to 4 millimeters tall and 0.5 to 2 millimeters wide. FIG. 44E is a lateral view of the embodiment of the invention drawn in FIG. 44D, the pointed end of a needle 4420, and a cable loop, which contains an elastic projection, such as a knot 4422.

FIG. 44F is a lateral view of the embodiment of the invention drawn in FIG. 44E. The elastic projection of the cable loop became stuck in the projections from the sleeve as the needle was pulled away from the sleeve. When used in discs, the sleeve is pulled from the disc, which pulls the cable loop through the surgically created aperture and the fissure, after the needle insertion tool is pulled from the disc. The invention enables surgeons to pull cable loops through portions of the AF, without direct observation of such areas of the AF. The invention also eliminates the need for surgeons to pull cable loops with additional tools, such as hooks. FIG. 44G is a lateral view of an alternative embodiment of the invention drawn in FIG. 44F. The projection is connected to two areas 4434, 4432 of the distal end of the sleeve 4430.

FIG. 45 is a superior view of the distal end of an alternative embodiment of the invention drawn in FIG. 44C. A cable loop 4504 with an elastic projection is seen in a slot of a needle 4502. The projection from the cable loop may lie in a recess on the side of the needle, near the slot. Projections are seen on the distal end of the footplate of the tool. The pointed end of the needle is pushed through AF tissue then past the projections on the footplate. The needle pulls the elastic projection of the cable past the footplate. The elastic projection from the cable loop is trapped by the projections on the footplate, when the needle is pulled from the disc.

FIG. 46A is a superior view of an alternative embodiment of the invention drawn in FIG. 38A and an axial cross section of a portion of the AF. The distal end 4604 of a flexible longitudinal component 4602 is seen extending through a surgically created aperture lateral to a fissure in the AF. For example, the distal end of the flexible longitudinal component could be passed through a needle-like tool that created the surgical aperture. The distal end of the flexible longitudinal component is seen in its first, elongate shape.

FIG. 46B is a superior view of the embodiment of the invention and the portion of the AF drawn in FIG. 46A. The distal end of the flexible longitudinal component is seen in its radially expanded shape. The lateral end of the radially expanded device extends beyond the edge of the fissure 4600. Instruments are passed through the fissure to grasp the radially expanded component in the next step of the procedure. The radially expanded device is preferably 4 to 15 millimeters wide. The elongate device is preferably 8 to 20 millimeters long and 0.8 to 2.0 millimeters wide. Tension on a central flexible longitudinal component may expand the distal end of the device. Alternatively, elastic or shape memory components may undergo such expansion as they are forced from the needle-like tool. The expandable component is preferably made of metal, including shape memory metals such as Nitinol, or plastic. Alternatively, a flexible longitudinal component fastened to an expandable balloon could be used in alternative embodiments of the invention.

FIG. 47A is a superior view of an alternative embodiment of the invention drawn in FIG. 46A and an axial cross section of a portion of the AF. The elastic component at the distal end of a flexible longitudinal component was passed through a surgically created aperture with needle-like tool. The elastic component is seen in its first shape.

FIG. 47B is a superior view of the embodiment of the invention and the portion of the AF drawn in FIG. 47A. The elastic component 4702 is seen in its expanded shape, which it assumes after it is released from the needle. The expanded elastic component is preferably 5 to 15 millimeters wide and extends beyond one edge of the fissure. The restrained component is preferably 5 to 20 millimeters long and 0.8 to 2.0 millimeters wide. The component is preferably made of the materials listed in other embodiments of the invention, including those depicted in FIG. 46B.

FIG. 48A is a superior view of the distal end of an alternative embodiment of the invention drawn in FIG. 22A. The slot 4804 in the near the pointed end of the needle leads 4802 to an eyelet-like feature 4806 in the needle.

FIG. 48B is a superior view of the embodiment of the invention drawn in FIG. 48A and a flexible longitudinal component 4810. The flexible longitudinal component 4810 is pulled into the slot in the needle, which bends the elastic component in the needle and allows the flexible longitudinal component to enter the eyelet-like feature. The elastic component returns to the position shown in FIG. 48A after the flexible longitudinal component is pulled into the eyelet, which traps the flexible component in the eyelet of the needle. The needle is preferably made of elastic metal, such as stainless steel, titanium, or Nitinol.

FIG. 48C is a superior view of the distal end of an alternative embodiment of the invention drawn in FIG. 48A. The elongate area 4814 of the needle 4812 lateral to the eyelet bends as the flexible longitudinal component is pulled into the eyelet then the elongate area of the needle returns to its resting position to hold the flexible longitudinal component in the eyelet of the needle. FIG. 48D is a superior view of the distal end of an alternative embodiment of the invention drawn in FIG. 48C. The elongate area 4824 of the needle 4822 lateral to the eyelet bends ad the flexible longitudinal component is pulled into the eyelet then the elongate area of the needle returns to its resting position to hold the flexible longitudinal component in the eyelet of the needle.

FIG. 49A is a superior view of alternative embodiments of the inventions drawn in FIGS. 6L and 11F. A mesh, braided, woven, or porous sleeve 4902 is seen surrounding a flexible elongate longitudinal fixation component 4910. The sleeve is preferably 0.5 to 10 millimeters in diameter and 2 to 30 millimeters long. For example, the sleeve could preferably be 5 millimeters in diameter, 20 millimeters long, and made of polyester woven polyester fibers. The sleeve preferably has pores 0.1 to 2 millimeters in diameter or width.

FIG. 49B is a superior view of the embodiment of the invention drawn in FIG. 49A and an axial cross section of a portion of the AF. The ends of the flexible longitudinal fixation component 4910 were passed through AF tissue lateral to the fissure using the previous embodiments of the invention then the sleeve 4902 was pushed through the fissure 4920. The sleeve is longer “L” than the width “W” of the fissure and preferably longer than the distance between the vertical arms of the flexible longitudinal fixation component. The flexible longitudinal fixation components are preferably passed through AF tissue 2 to 4 millimeters cranial or caudal to horizontal fissures in the AF and 4 to 8 millimeters lateral to vertical fissures. Flexible longitudinal fixation components placed too close to fissures, for example less than 2 to 3 millimeters, may be easily pulled through the AF tissue. Tension on the ends of flexible longitudinal fixation components placed too far from fissures, for example 4 to 9 millimeters, depending on the orientation of the fissure, does not pull the edges of fissure together.

FIG. 49C is a superior view of the embodiment of the invention and axial cross section of the portion of the AF drawn in FIG. 49B. Tension on the ends of the flexible longitudinal fixation component folds the sleeve, somewhat like an accordion contracts, then the ends of the flexible longitudinal component are welded 4912. Two or more disc-shaped porous components could be threaded over the flexible longitudinal fixation component in alternative embodiments of the invention. The flexible longitudinal fixation component is preferably passed through the centers of such disc-like components. The disc-like components are preferably 3 to 14 millimeters in diameter and 0.2 to 1 millimeter thick. The assembled disc components are preferably the same size as the sleeve described in the text of FIG. 49A and preferably made of the same materials described in the text of FIG. 49A.

FIG. 50A is a superior view of an alternative embodiment of the invention drawn in FIG. 44A and an axial cross section of a portion of the AF. The distal end 5006 of the guide component 5002 is seen in a fissure in the AF. Two needles 5010, 5012 pass through the guide component and the AF. A loop of a flexible longitudinal fixation component 5020 is seen at the top of the drawing. The distance between the needles is preferably between 6 and 10 millimeters. The needles are preferably 1 to 2.5 millimeters in diameter and about 15 to 30 centimeters long. The proximal ends of the needles may be housed in handle components. The distal end of the guide component is preferably 2 to 4 millimeters in diameter or width and about 4 to 10 millimeters long. The guide component is preferably made of plastic.

FIG. 50B is a superior view of the embodiment of the invention and axial cross section of the portion of the AF drawn in FIG. 50A. A component made of shape memory material was pushed distally relative to the guide component. The distal ends 5030, 5032 of the guide component are seen extending towards the needles. The ends 5022, 5024 of the flexible longitudinal fixation component are releasably fastened to the ends of the shape memory component. The shape memory component is preferably made of Nitinol or other shape-memory material. The distal ends of the shape memory component are preferably 0.5 to 1.5 millimeters in diameter and 4 to 15 millimeters long.

FIG. 50C is a superior view of the embodiment of the invention and axial cross section of the portion of the AF drawn in FIG. 50B. The ends of the shape memory component and the flexible longitudinal fixation component were advanced through holes in the sides of the needles 5010, 5012. Elastic projections 5014, 5016 attached to the ends of the flexible longitudinal fixation component are seen expanded and resting against the lateral sides of the needles.

FIG. 50D is a superior view of the embodiment of the invention and axial cross section of the portion of the AF drawn in FIG. 50C. The shape memory component was pulled from the guide component. The elastic projections 5014, 5016 hold the ends 5022, 5024 of the flexible longitudinal fixation component 5020 in the needles as the shape memory component is pulled from the guide component. Stylets in the needles were pulled in a proximal direction relative to the needles to capture the ends of the flexible longitudinal fixation components in the needles 5010, 5012.

FIG. 50E is a superior view of the embodiment of the invention and axial cross section of the portion of the AF drawn in FIG. 50D. The needles and guide component are pulled away from the IVD relative to FIG. 50D.

FIG. 50F is a superior view of the embodiment of the invention and axial cross section of the portion of the AF drawn in FIG. 50E. The needles and guide component were pulled from the IVD. The ends of the flexible longitudinal fixation component are pulled further away from the IVD in the next step of the procedure, which pulls the central portion of the flexible fixation component into the IVD, through the fissure. The ends of the flexible fixation component are released from the needles and welded in the next step of the procedure.

FIG. 50G is a superior view of a longitudinal cross section of the embodiment of the invention drawn in FIG. 50F. Projections from the end of the components fastened to the flexible fixation component are seen in openings at the ends of the shape memory component. The opening 5042 and stylet 5040 are seen in one of the needles.

FIG. 50H is a superior view of a longitudinal cross section of the embodiment of the invention drawn in FIG. 50G. The stylet was pulled in a proximal direction relative to the needle to capture the flexible fixation component after the shape memory component was pulled from the needle

FIG. 50I is a lateral view of a longitudinal cross section of the embodiment of the invention drawn in FIG. 50H. An opening 5052 is seen on the side of the stylet component 5050.

FIG. 51 is a superior view of an alternative embodiment of the invention drawn in FIG. 50G and an axial cross section of the portion of the AF. The distal end of a shape memory component 5104, to which the end of a flexible longitudinal fixation component 5102 was fastened, was advanced from one needle 5110, through the distal end of the guide component and into an opening in the side of a second needle 5112. The guide component helps guide the needle into the opening in the second needle. The shape memory component is pulled from the first needle in the next step of the procedure. An elastic projection 5114 from the component on the end of the flexible fixation component and the stylet hold the flexible fixation component in the second needle. The second needle is pulled from the IVD and the flexible fixation member is pulled through an opening in the distal end of the guide component in the next step of the procedure. The ends of the flexible longitudinal fixation component are then released and welded.

FIG. 52A is a superior view of a longitudinal cross section of an alternative embodiment of the invention drawn in FIG. 51. The distal end of an elastic, shape memory component 5200, which releasably holds the end of flexible longitudinal fixation component 5202 is seen extending from first needle and through a rotating guide component 5204. FIG. 52B is a superior view of a longitudinal cross section of the embodiment of the invention drawn in FIG. 52A. The distal end of the guide component was rotated to the second needle as the shape memory component was advanced into the IVD and the opening in the second needle. The guide component directs 5204 the distal end of the shape memory component 5200 into the opening in the side of the second needle 5210. The two piece guide component rotates about two axes, 5220, 5222. The shape memory component is removed from the IVD and the end of the flexible fixation component pulled from the IVD in the next step of the procedure. The ends of the flexible fixation components are then welded together.

1. Apparatus for repairing a defect in an anulus fibrosis surrounding an intradiscal space, the apparatus comprising:
a first surgical instrument having a distal end adapted for penetration through an annulus fibrosis on one side of a defect in the annulus fibrosis such that the distal end of the first surgical instrument enters into an intradiscal space;
a flexible longitudinal fixation component temporarily coupled to the distal end of the first surgical instrument; and
a second surgical instrument having a distal end adapted for penetration through an annulus fibrosis on the other side of a defect in the annulus fibrosis or through a defect in the annulus fibrosis, the distal end of the second surgical instrument including a device for capturing the flexible longitudinal fixation component within an intradiscal space and pulling the flexible longitudinal fixation component so as to repair the defect.

2. The apparatus of claim 1, wherein:
the flexible longitudinal fixation component is attached to a magnet or magnetic material; and
the distal end of the second surgical instrument includes a magnet or magnetic material operative to capture the flexible longitudinal fixation component within an intradiscal space by way of magnetic attraction.

3. The apparatus of claim 1, wherein the distal end of the second surgical instrument includes a mechanism that grabs the flexible longitudinal fixation component.

4. The apparatus of claim 1, wherein the distal end of the second surgical instrument includes a plurality of barbs or projections grab the flexible longitudinal fixation component.

5. The apparatus of claim 1, wherein the distal end of the second surgical instrument includes a needle for capturing the flexible longitudinal fixation component.

6. The apparatus of claim 1, wherein the distal end of the second surgical instrument includes a scissoring mechanism that grabs the flexible longitudinal fixation component.

7. The apparatus of claim 1, wherein the instrument, the flexible longitudinal fixation component, or both, include an anti-backout structure.

8. The apparatus of claim 1, wherein the flexible longitudinal fixation component is a length of suture material.

9. A method of repairing a defect in an anulus fibrosis having multiple layers surrounding an intradiscal space, comprising the steps of:
forcing a flexible longitudinal fixation component through all layers of an anulus fibrosis and into an intradiscal space on one side of a defect in the annulus fibrosis;
penetrating all layers of an anulus fibrosis on the other side of a defect in the annulus fibrosis, or through a defect in the annulus fibrosis, with a surgical instrument having a distal end with a device for capturing the flexible longitudinal fixation component within an intradiscal space;
capturing the flexible longitudinal fixation component within an intradiscal space; and
pulling the flexible longitudinal fixation component back out and through all layers of an anulus fibrosis so as to repair the defect.

10. The method of claim 9, wherein:
the flexible longitudinal fixation component is attached to a magnet or magnetic material; and
the step of capturing the flexible longitudinal fixation component within an intradiscal space includes magnetic attraction.

11. The method of claim 9, wherein the step of capturing the flexible longitudinal fixation component within an intradiscal space includes grabbing the flexible longitudinal fixation component with a surgical instrument.

12. The method of claim 9, wherein the flexible longitudinal fixation component is a length of suture material.
